(19) United States
(12) Patent Application Publication
(10) Pub. No.: US 2016/0199434 A1
(43) Pub. Date: Jul. 14, 2016

(54) COMPOSITIONS AND METHODS FOR MODULATING DNA METHYLATION

(71) Applicant: OHIO STATE INNOVATION FOUNDATION, Columbus, OH (US)

(72) Inventors: Timothy Darren Eubank, Columbus, OH (US); Clay Braden Marsh, Columbus, OH (US); Duaa Dakhlallah, Columbus, OH (US)

(73) Assignee: Ohio State Innovation Foundation, Columbus, OH (US)

(21) Appl. No.: 14/911,380

(22) PCT Filed: Aug. 15, 2014
(86) PCT No.: PCT/US2014/051317

§ 371 Date: Feb. 10, 2016

Related U.S. Application Data
(60) Provisional application No. 61/867,000, filed on Aug. 16, 2013.

(51) Int. Cl. A61K 38/05; C12Q 1/68; A61K 9/00; A61K 45/06

(52) U.S. Cl. CPC A61K 38/05; A61K 45/06; C12Q 1/6886; A61K 9/0019; A61K 9/0053; C12Q 1/6883; C12Q 2600/154

(57) ABSTRACT

Disclosed herein are compositions and methods for modulating DNA methylation of one or more gene promoters, treating a subject diagnosed with or suspected of having a disease or disorder characterized by DNA hypermethylation, decreasing c-myc expression, increasing desmoplakin expression, and inhibiting metastases. This abstract is intended as a scanning tool for purposes of searching in the particular art and is not intended to be limiting of the present invention.

### CROSS-REFERENCE TO RELATED APPLICATION

This application claims the priority of and the benefit of the filing date of U.S. Provisional Patent Application No. 61/867,000, filed Aug. 16, 2013, which is herein incorporated in its entirety.

### BACKGROUND

DNA methylation generally describes the process of turning off gene expression. With long stretches of Cs (cytosine) and Gs (guanine) in the promoters of some genes (called “CpG islands”), these regions are susceptible to DNA methylation, which is a process in which a methyl group is added onto the 5′ position of Cs. The presence of the methyl group on the C inhibits the transcriptional machinery necessary to express the gene.

DNA Methyltransferases (DNMTs) and histone deacetylases (HDACs) regulate the methylation process. In normal physiology, DNA methylation is a transient event, meaning that DNMTs and HDACs work to add methyl groups to Cs while DNA methylases (DNMs) and histone methyltransferases (HATs) work to remove them. In normal cells, DNA methylation occurs predominantly in repetitive genomic regions, including satellite DNA and parasitic elements (such as long interspersed transposable elements (LINES), short interspersed transposable elements (SINES) and endogenous retroviruses) (Yoder et al., 1997). CpG islands, particularly those associated with promoters, are generally unmethylated. DNA methylation represses transcription directly, by inhibiting the binding of specific transcription factors, and indirectly, by recruiting methyl-CpG-binding proteins and their associated repressive chromatin remodeling activities.

Properly established and maintained DNA methylation patterns are essential for mammalian development and for the normal functioning of the adult organism as well as aging. DNA methylation is a potent mechanism for silencing gene expression and maintaining genome stability in the face of a vast quantity of repetitive DNA, which can otherwise mediate illegitimate recombination events and cause transcriptional deregulation of nearby genes.

There is still a scarcity of compositions and methods that can effectively modulate DNA methylation. These needs and other needs are satisfied by the present invention.

### SUMMARY

Disclosed herein is a composition for modulating DNA methylation of one or more gene promoters, comprising an effective amount of a compound of the formula:

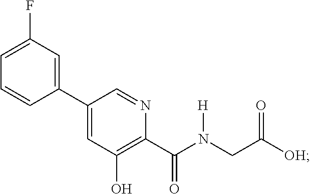 [SMILES: O=C(O)CNC(=O)c1ncc(-c2cccc(F)c2)cc1O]

or a pharmaceutically acceptable salt thereof.

Disclosed herein is a composition for treating a subject diagnosed with or suspected of having a disease or disorder characterized by DNA hypermethylation, comprising an effective amount of a compound of the formula:

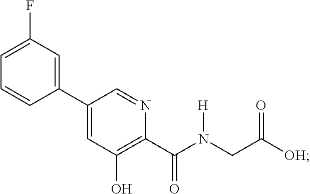 [SMILES: O=C(O)CNC(=O)c1ncc(-c2cccc(F)c2)cc1O]

or a pharmaceutically acceptable salt thereof.

Disclosed herein is a composition for decreasing c-Myc expression, comprising an effective amount of a compound of the formula:

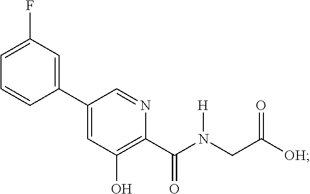 [SMILES: O=C(O)CNC(=O)c1ncc(-c2cccc(F)c2)cc1O]

or a pharmaceutically acceptable salt thereof.

Disclosed herein is a composition for increasing desmoplakin, comprising an effective amount of a compound of the formula:

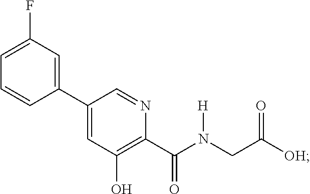 [SMILES: O=C(O)CNC(=O)c1ncc(-c2cccc(F)c2)cc1O]

or a pharmaceutically acceptable salt thereof.

Disclosed herein is a composition for inhibiting metastases, comprising an effective amount of a compound of the formula:

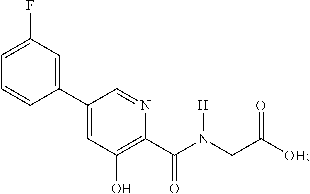 [SMILES: O=C(O)CNC(=O)c1ncc(-c2cccc(F)c2)cc1O]

or a pharmaceutically acceptable salt thereof.

Disclosed herein is a composition for treating cancer, comprising an effective amount of a compound of the formula:

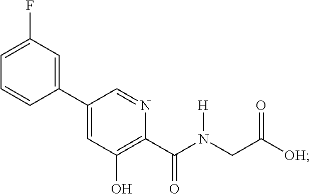 [SMILES: O=C(O)CNC(=O)c1ncc(-c2cccc(F)c2)cc1O]

or a pharmaceutically acceptable salt thereof; and one or more chemotherapeutic agents.

Disclosed herein is a composition for treating cancer, comprising an effective amount of a compound of the formula:

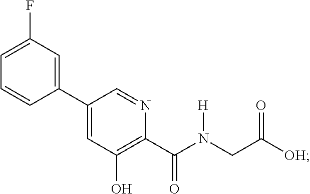 [SMILES: O=C(O)CNC(=O)c1ncc(-c2cccc(F)c2)cc1O]

a pharmaceutically acceptable salt thereof; and one or more anti-cancer agents.

Disclosed herein is a method of modulating DNA methylation of one or more gene promoters in a subject, comprising administering to a subject an effective amount of composition comprising a compound of the formula:

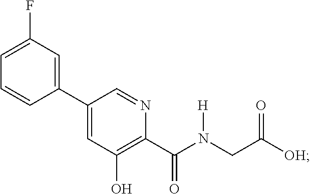 [SMILES: O=C(O)CNC(=O)c1ncc(-c2cccc(F)c2)cc1O]

or a pharmaceutically acceptable salt thereof.

Disclosed herein is a method of modulating DNA methylation of one or more gene promoters in a subject, comprising identifying a subject in need of treatment by determining the methylation status of one or more gene promoters; and administering to a subject an effective amount of composition comprising a compound of the formula:

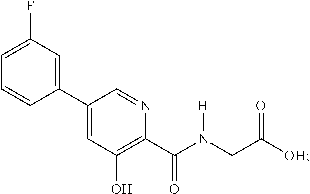 [SMILES: O=C(O)CNC(=O)c1ncc(-c2cccc(F)c2)cc1O]

or a pharmaceutically acceptable salt thereof.

Disclosed herein is a method of treating a subject diagnosed with or suspected of having a disease or disorder characterized by DNA hypermethylation, comprising administering to a subject an effective amount of a composition comprising a compound of the formula:

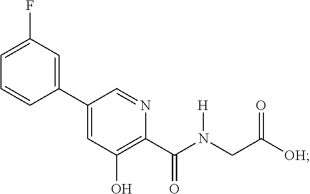 [SMILES: O=C(O)CNC(=O)c1ncc(-c2cccc(F)c2)cc1O]

or a pharmaceutically acceptable salt thereof.

Disclosed herein is a method of decreasing c-myc expression in a subject, comprising administering to a subject an effective amount of a composition comprising a compound of the formula:

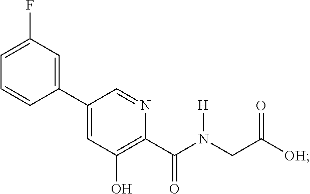 [SMILES: O=C(O)CNC(=O)c1ncc(-c2cccc(F)c2)cc1O]

or a pharmaceutically acceptable salt thereof.

Disclosed herein is a method of increasing desmoplakin expression in a subject, comprising administering to a subject an effective amount of a composition comprising a compound of the formula:

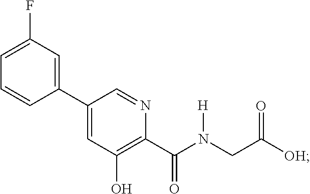 [SMILES: O=C(O)CNC(=O)c1ncc(-c2cccc(F)c2)cc1O]

or a pharmaceutically acceptable salt thereof.

Disclosed herein is a method of inhibiting metastases in a subject, comprising administering to a subject an effective amount of a composition comprising a compound of the formula:

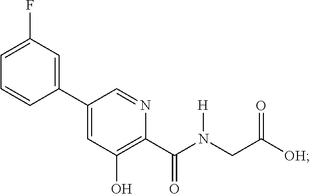 [SMILES: O=C(O)CNC(=O)c1ncc(-c2cccc(F)c2)cc1O]

or a pharmaceutically acceptable salt thereof; and modulating the DNA methylation status of one or more genes promoters.

### BRIEF DESCRIPTION OF THE FIGURES

The accompanying Figures, which are incorporated in and constitute a part of this specification, illustrate several aspects of the invention and together with the description serve to explain the principles of the invention.

FIGS. 1A-1E show DNA methyltransferase mRNA expression in vitro and in vivo. FIG. 1A shows that when compared to DMSO, AKB-6899 (i) significantly inhibited DNMT-1 at 10 μM, (ii) significantly inhibited DNMT-1 and DNMT-3a at 25 μM, and (iii) significantly inhibited DNMT-1 and DNMT-3a at 50 μM. PyMT tumor cells were left untreated, were treated with DMSO, or were treated with AKB-6899 (10, 25, or 50 μM) for 24 hours. The cells were subjected to Trizol extraction to recover total RNA. cDNA was synthesized for RT-PCR of DNMT-1 and DNMT-3a mRNA expression. FIG. 1B shows that when compared to DMSO, AKB-6899 (25 μM) significantly decreased the expression of both DNMT-1 and DNMT-3a. Human MDA-MB-231 tumor cells were left untreated, were treated with DMSO, or were treated with AKB-6899 (25 μM) for 24 hours. Total RNA was isolated for RT-PCR. FIG. 1C (DNMT-1), FIG. 1D (DNMT-3a), and FIG. 1E (DNMT-3b) compare the effect of AKB-6899 with the effect of 5-aza-2′deoxycytidine (5-AZA) as assessed by RT-PCR in human cervical tumors implanted in mice that were either untreated (UTX), treated with DMSO, treated with 5-AZA, or treated with AKB-6899 (17.5 mg/kg).

FIGS. 2A-2D show HDAC expression in vitro and in vivo. FIG. 2A (grouped by HDAC) and FIG. 2B (grouped by treatment) both show the expression of HDAC 1-HDAC10 in a human cervical cancer cell line (HeLa). Cells were left untreated, were treated with DMSO, were treated with AKB-6899 (5 or 50 μM), or were treated with phenylbutyrate (PBA), which has been considered to the “gold standard” of HDAC inhibitors. FIG. 2C shows HDAC1 expression in human cervical tumors in mice following treatment with DMSO, 5-aza-2′deoxycytidine (decitabine), or AKB-6899. FIG. 2D shows HDAC3 expression in human cervical cancer tumors following treatment with DMSO, 5-AZA, or AKB-6899 (17.5 mg/kg mouse weight).

FIGS. 3A-3E show the effect of AKB-6899 (25 μM) on methylation of the desmoplakin (DSP) gene promoter in PyMT breast cancer cells (3A), human MDA-MB-231 triple negative breast cancer cells (3B), human C8161.9 melanoma cells (3C), and human MCF-7 breast cancer cells (3D). In FIG. 3A, the DSP promoter was 100% unmethylated (AKB-6899) vs. 99.99% methylated (vehicle). In FIG. 3B, the DSP promoter was 99.62% unmethylated (AKB-6899) v. 99.89% (vehicle). In FIG. 3C, the DSP promoter was 99.90% unmethylated (AKB-6899) v. 42.04% (vehicle). In FIG. 3D, the DSP promoter was 92.48% unmethylated (AKB-6899) v. 98.48% (vehicle). In FIG. 3E, when compared to the vehicle (DMSO), treatment of PyMT cells with AKB-6899 increased DSP expression.

FIGS. 4A-4B show desmoplakin mRNA expression in vitro and in vivo. FIG. 4A shows cultured PyMT cells following treatment with DMSO or AKB-6899 (10 μM). Compared to DMSO, AKB-6899 significantly increases desmoplakin mRNA expression. FIG. 4B shows desmoplakin expression from PyMT tumors (in vivo). Compared to the vehicle, AKB-6899 significantly increased desmoplakin expression.

FIGS. 5A-5B show desmoplakin protein expression following AKB-6899 treatment. In FIG. 5A, cultured PyMT tumor cells were treated for 24 hours with DMSO or AKB-6899 (10 μM). AKB-6899 increased desmoplakin protein expression. In FIG. 5B, desmoplakin protein expression was measured in human MDA-MB-231 cells, which do not expression desmoplakin.

FIGS. 6A-6B show that percent CpG methylation of the c-Myc promoter in FIG. 6A and for DSP promoter in FIG. 6B following various treatments. HeLa cervical cancer cells were left untreated, were treated with DMSO, were treated with 5-AZA, were treated with AKB-6899 (5 or 25 μM), or were treated with PBA (2 mM). As compared to DMSO and PBA, AKB-6899 precipitated an increase in demethylation of these two promoters.

FIGS. 7A-7B show that percent DNA methylation of the c-Myc promoter in 7A and the relative copy number of the c-Myc promoter in 7B following various in vivo treatments. Animal models of human cervical tumors were untreated, treated with DMSO, treated with 5-AZA or treated with AKB-6899 (17.5 mg/kg). AKB-6899 appears to have completely demethylated the c-Myc promoter and inhibited c-Myc expression.

FIGS. 8A-8B show the cytotoxicity of AKB-6899. In FIG. 8A, HeLa cells were treated with AKB-6899, Docetaxel, or a combination of AKB-6899 and Docetaxel at various concentrations. An XTT assay was used to determined percent survival. In FIG. 8B, MDA-MB-231 cells were treated with AKB-6899, Docetaxel, or a combination of AKB-6899 and Docetaxel at various concentrations. An XTT assay was used to determined percent survival.

FIG. 9 shows the effects of various treatments on human A375 melanoma cells that had been implanted in SCID mice. During treatment, tumor growth was determined. Animals were treated with one the following combinations: vehicle/vehicle, vehicle/AKB-6899 (17.5 mg/kg), Docetaxel (30 mg/kg)/vehicle, or Docetaxel/AKB-6899. 19 days after palpable tumor formation, the tumors in the mice receiving the vehicle/AKB-6899 combination had tumors that were 74% the size of control; the tumors in the mice receiving Docetaxel/vehicle combination had tumors that were 49% the size of control; and the tumors in the mice receiving the Docetaxel/AKB-6899 combination had tumors that were 13% the size of control.

FIGS. 10A-10B show that AKB-6899 inhibited DNMT transcription in a dose dependent manner. In FIG. 10A, AKB-6899 inhibited DNMT-1, DNMT-3a, and DNMT-3b transcription in a dose-dependent manner in human squamous cell carcinoma lung cancer cells (H1703), in vitro. The DMSO-treated DNMT mRNAs are over 1.0, meaning the level of expression is higher than the housekeeping control gene, CAP1. In FIG. 10B, AKB-6899 inhibited DNMT-3a and DNMT-3b transcription in human adenocarcinoma lung cancer cells (A549). The DMSO-treated DNMT mRNAs are under 1.0, meaning the level of expression is lower than the housekeeping control gene, CAP1.

FIGS. 11A-11D show the percent methylation for 23 different gene promoters in two types of human lung cancer cells—FIGS. 11A and 11B are directed at H1703 squamous cell carcinoma and FIG. 11C and FIG. 11D are directed at A549 adenocarcinoma. DMSO was used as a vehicle and was used as a control. As compared to the DMSO-treated control group, AKB-6899 (10 μM and 50 μM) decreased the percent of DNA methylation in a dose response fashion. The Dec/PBA condition represents a combination of 5 μM Decitabine (5-aza-2′deoxycytidine (5-AZA), which is a demethylating agent) and 2 mM phosphobutyric acid (PBA, HDAC inhibitor). In all promoters tested, and in both types of lung cancer, AKB-6899 demethylated the gene promoters such that the level of % DNA methylation following AKB-6899 treatment was below the level of % DNA methylation of the DMSO-treated control cells (DMSO).

FIGS. 12A-12D show the level of mRNA expression for a panel of 14 common tumor suppressor genes in two types of human lung cancer cells (i.e., H1703 squamous cell carcinoma in FIG. 12A and A549 adenocarcinoma in FIG. 12B). AKB-6899 and Dec/PBA were more effective at inducing mRNA expression in H1703 squamous cell carcinoma than in the A549 adenocarcinoma. All of the tumor suppressor mRNAs were augmented in response to AKB-6899 but one, the CDKN1C gene in A549 adenocarcinoma cells, in which expression levels were 0.1 (AKB10) and 0.9 (AKB50) compared to DMSO standardized to 1.0. In FIGS. 12A-12B, the following apply: “1”=DMSO, “2”=AKB-6899 (10 μM), “3”=AKB-6899 (50 μM), and “4”=combination of Decitabine (5 μM)/PBA (2 mM). FIGS. 12C and 12D are raw values for mRNA expression of these 14 genes.

FIGS. 13A-13B show XTT cell survival assays for normal lung epithelial cells (BEAS-2B) and lung cancer cells (H1703 squamous cell carcinoma and A549 adenocarcinoma) in response to increasing doses of AKB-6899. In FIG. 13A, even at the highest dose, no significant decrease in the survival of BEAS-2B cells was detected. In FIG. 13B, there was a pronounced toxicity of the two lung cancer cells following increasing doses of AKB-6899.

FIGS. 14A-14C show the effects of systemic administration of AKB-6899. Mice were treated three times per week for three weeks with either i.p. AKB-6899 or i.p. DMSO. The lungs were removed and assayed to determine DNMT mRNA expression, percent CpG methylation, and fold change in mRNA. The data show that AKB-6899 down-regulated the expression of the DNMTs and decreased DNA methylation. FIG. 14A show that, when compared to DMSO treatment, AKB-6899 (17.5 mg/kg) inhibited expression of DNMT-1, DNMT-3a, and DNMT-3b in the lungs of normal mice. FIG. 14B shows that AKB-6899 demethylated the DSP and RASSF1 promoters in the lungs of these mice. FIG. 14C shows that as a result of the demethylation described in FIG. 14B, the level of DSP and RASSF1 mRNA expression increased. The data was standardized to 1.0 by the DMSO levels of the respective gene expression, (n=4 lungs per condition).

FIG. 15 shows RNAseq data following administration of AKB-6899 to MDA-MB-231 cancer cells (as compared to DMSO-treated MDA-MB-231 cancer cells.

Additional advantages of the invention are set forth in part in the description that follows, and in part will be obvious from the description, or can be learned by practice of the invention. It is to be understood that both the foregoing general description and the following detailed description are exemplary and are not restrictive of the invention as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. DEFINITIONS

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein -disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. For example, in an aspect, a disclosed composition comprising a disclosed composition, such as, for example, AKB-6899, can optionally comprise one or more other agents, such as, for example, anti-cancer agents or anti-proliferation agents or anti-methylation agents or chemotherapeutic agents.

As used herein, the term “analog” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as a disclosed compounds, such as, for example, ABK-6899.

As used herein, “homolog” or “homologue” refers to a polypeptide or nucleic acid with homology to a specific known sequence. Specifically disclosed are variants of the nucleic acids and polypeptides herein disclosed that have at least 40, 41, 42, 43, 44, 45, 46, 47, 48, 49, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated or known sequence. In an aspect, specifically disclosed nucleic acids and polypeptides include the following: APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A. In an aspect, specifically disclosed nucleic acids and polypeptides include the following: HIF, DNMT-1, DNMT-3a, DNMT-3b, PHD1, PHD2, PHD3, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7. Those of skill in the art readily understand how to determine the homology of two proteins or nucleic acids. For example, the homology can be calculated after aligning the two sequences so that the homology is at its highest level. It is understood that one way to define any variants, modifications, or derivatives of the disclosed genes and proteins herein is through defining the variants, modification, and derivatives in terms of homology to specific known sequences.

As used herein, the term “subject” refers to the target of administration, e.g., an animal. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered.

A subject can be an unhealthy subject. In other words, a subject can be not healthy. For example, a subject can also be afflicted with one or more diseases or disorders. A disease or disorder can be a disease or disorder characterized by hypermethylation of one or more gene promoters. In an aspect, a disease or disorder can be cancer or a tumor or aberrant cell growth. In an aspect, a tumor can be a solid tumor. In an aspect, a tumor can be not a solid tumor. In an aspect of a disclosed method, a subject can be diagnosed with a need for treatment of one or more of the aforementioned diseases or disorders prior to the administering step. In an aspect of a disclosed method, a subject can be diagnosed with a need for inducing apoptosis of malignant cells, such as, for example, malignant cancer cells. In an aspect of a disclosed method, a subject can be diagnosed with a need for modulating DNA methylation of one or more gene promoters. In an aspect of a disclosed method, a subject can be diagnosed with a need for altering the methylation status of one or more gene promoters, such as, for example, by reducing the percent methylation of one or more gene promoters. In an aspect, a subject can have a disease or disorder that is not vascular leak. In an aspect, a subject can have a disease or disorder that is not retinopathy. In an aspect, a subject can have a disease or disorder that is not critical limb ischemia (CLI).

As used herein, a “patient”’ can be a subject, such as, for example, a human subject.

As used herein, the term “treatment” refers to the medical management of a patient with the intent to cure, ameliorate, stabilize, or prevent a disease, pathological condition, or disorder (such as, for example, a disease or disorder characterized by DNA hypermethylation of one or more gene promoters) This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease or disorder, pathological condition, or disorder. In various aspects, the term covers any treatment of a subject, including a mammal (e.g., a human), and includes: (i) preventing the disease or disorder from occurring in a subject that can be predisposed to the disease or disorder but has not yet been diagnosed as having it; (ii) inhibiting the disease or disorder i.e., arresting its development; or (iii) relieving the disease or disorder, i.e., causing regression of the disease or disorder, or relieving the symptoms associated with the disease or disorder.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing malignant cell growth is intended. In an aspect, inhibiting or diminishing or decreasing the percent methylation of one or more gene promoters is disclosed. In an aspect, altering or modulating the methylation status of one or more gene promoters is disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” and “having cancer” mean having been subjected to a physical examination by a person of skill, for example, a physician, and found to have a condition that can be diagnosed or can be treated by a compound or composition that can prevent or inhibit malignant cell growth and/or can induce apoptosis in a population of cells, such as cancer cells or tumor cells. As a further example, “diagnosed with a disease or disorder characterized by hypermethylation” refers to having been subjected to a physical examination by a person of skill, for example, a physician, and found to have a condition characterized by hypermethylation of one or more gene promoters wherein modulating or altering the methylation status of one or more gene promoters would be beneficial to the subject. In an aspect, the subject benefits by decreasing the percent methylation of one or more gene promoters.

As used herein, the phrase “identified to be in need of treatment thereof,” or the like, refers to selection of a subject based upon need for treatment of a disease or disorder or illness or condition. The identified subject can be an unhealthy subject. For example, a subject can be identified as having a need for treatment of a disease or disorder (e.g., a disease or disorder characterized by hypermethylation of one or more gene promoters) based upon an earlier diagnosis by a person of skill and thereafter subjected to treatment for that disease or disorder. For example, a subject can be identified as having a need for treatment of a disorder (e.g., cancer or a solid tumor or a non-solid tumor or some other disorder related to malignant cell growth or a disorder requiring apoptosis of a population of cells) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who performed the examination or evaluation.

As used herein, “aberrant” DNA methylation can refer to hypermethylation, hypomethylation, or both. In an aspect, aberrant DNA methylation can refer to hypermethylation of one or more gene promoters. As known to the skilled person, hypermethylation can refer to when a gene promoter is methylated at a greater extent in a cell or tissue (i.e., an affected cell or tissue) relative to the methylation percent in normal cell or tissue (i.e., an unaffected cell or tissue). In an aspect, aberrant DNA methylation can effect one or more of the following gene promoters: APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A. In an aspect, aberrant DNA methylation can effect one or more of the following gene promoters: HIF, DNMT-1, DNMT-3a, DNMT-3b, PHD1, PHD2, PHD3, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7.

TABLE 1
List of Cancers in which DNA Hypermethylation is Involved
Cancers

Acute Lymphoblastic Leukemia
Acute Myeloid Leukemia
Adrenocortical Carcinoma
Adult Acute Myeloid Leukemia
Astrocytoma
Basal Cell Carcinoma
Bile Duct Cancer
Bladder Cancer
Bladder Cancer
Bone Cancer
Brain and Spinal Cord Tumors
Brain Stem Glioma
Breast Cancer
Bronchial Tumors
Burkitt Lymphoma
Carcinoid Tumor
Central Nervous System Embryonal Tumors
Central Nervous System Lymphoma
Cerebellar Astrocytoma
Cerebral Astrocytoma/Malignant Glioma
Cervical Cancer
Childhood Brain Stem
Childhood Cerebral
Chordoma
Chronic
Chronic Lymphocytic Leukemia
Chronic Myelogenous Leukemia
Chronic Myeloproliferative Disorders
Colon Cancer
Colorectal Cancer
Craniopharyngioma
Cutaneous T-Cell Lymphoma
Cutaneous T-Cell Lymphoma
Endometrial
Ependymoblastoma
Ependymoma
Esophageal Cancer
Ewing Family of Tumors
Extrahepatic
Extrahepatic Bile Duct Cancer
Eye Cancer
Gallbladder Cancer
Gastric (Stomach) Cancer
Gastrointestinal Cancer
Gastrointestinal Carcinoid Tumor
Gastrointestinal Carcinoid Tumor
Gastrointestinal Stromal Tumor (GIST)
Germ Cell Tumor
Gestational Trophoblastic
Glioma
Hairy Cell Leukemia
Head and Neck Cancer
Hepatocellular (Liver) Cancer
Histiocytosis
Hodgkin Lymphoma
Hypopharyngeal Cancer
Intraocular Melanoma
Intraocular Melanoma
Kaposi Sarcoma
Kidney (Renal Cell) Cancer
Langerhans Cell
Langerhans Cell Histiocytosis
Laryngeal Cancer
Leukemia
Lip and Oral Cavity Cancer
Liver Cancer
Lung Cancer
Malignant Fibrous Histiocytoma
Malignant Fibrous/Histiocytoma of Bone and Osteosarcoma
Medulloblastoma
Medulloepithelioma
Melanoma
Merkel Cell Carcinoma
Mesothelioma
Metastatic Squamous
Mouth Cancer
Multiple Endocrine Neoplasia Syndrome
Multiple Myeloma
Mycosis Fungoides
Myelodysplastic Syndromes
Myelodysplastic/Myeloproliferative Diseases
Myelogenous Leukemia
Myeloproliferative Disorder
Nasal Cavity
Nasopharyngeal Cancer
Neck Cancer with Occult Primary
Neuroblastoma
Non-Hodgkin Lymphoma
Non-small Cell Lung Cancer
Oral Cancer
Oral Cavity Cancer
Oropharyngeal Cancer
Osteosarcoma
Osteosarcoma and Malignant Fibrous Histiocytoma
Ovarian Cancer
Ovarian Epithelial Cancer
Ovarian Germ Cell Tumor
Pancreatic Cancer
Papillomatosis
Parathyroid Cancer
Penile
Pharyngeal
Pheochromocytoma
Pineoblastoma and Supratentorial Primitive Neuroectodermal Tumors
Pituitary Tumor
Plasma Cell Neoplasm/Multiple Myeloma
Primitive Neuroectodermal Tumors
Prostate
Rectal Cancer
Retinoblastoma
Rhabdomyosarcoma
Salivary Gland Cancer
Sézary Syndrome
Skin Cancer (Nonmelanoma)
Skin Carcinoma, Merkel Cell
Small Cell Lymphoma
Small Intestine Cancer
Soft Tissue Sarcoma
Squamous Neck
System Atypical Teratoid/Rhabdoid Tumor
T-cell Lymphoma
Throat Cancer
Thymoma and Thymic Carcinoma
Transitional Cell Cancer
Transitional Cell Cancer of the Renal Pelvis and Ureter
Trophoblastic Tumor
Tumor
Uterina Sarcoma
Uterine
Vaginal Cancer
Visual Pathway and Hypothalamic Glioma
Vulvar Cancer
Waldenström Macroglobulinemia
Wilms Tumor

TABLE 2
List of Cancers in which DNA Hypomethylation is Involved

Appendix Cancer
Central Nervous
Islet Cell Tumors
Pleuropulmonary Blastoma
Throat Cancer
Thyroid Cancer
Ureter
Wilms Tumor

TABLE 3
List of Cancers in which DNA Hypermethylation May Be Involved

AIDS-related Lymphoma
Childhood Visual Pathway and Hypothalamic
Extracranial Germ Cell Tumor
Extragonadal Germ Cell Tumor
Hypothalamic and Visual Pathway Glioma
Pineal Parenchymal Tumors of Intermediate Differentiation
Primary Central Nervous System Lymphoma
Respiratory Tract Carcinoma Involving the NUT Gene on Chromosome 15
Testicular

Methylation-specific PCR” or “MSP” can refer to an assay that entails initial modification of DNA by sodium bisulfite, converting all unmethylated, but not methylated, cytosines to uracil, and subsequent amplification with primers specific for methylated versus unmethylated DNA. MSP is known to the art to be a simple, quick and cost-effective method to analyze the DNA methylation status of virtually any group of CpG sites within a CpG island. The technique comprises two parts: (1) sodium bisulfite conversion of unmethylated cytosine's to uracil under conditions whereby methylated cytosines remains unchanged and (2) detection of the bisulfite induced sequence differences by PCR using specific primer sets for both unmethylated and methylated DNA. MSP requires only small quantities of DNA, is sensitive to 0.1% methylated alleles of a given CpG island locus, and can be performed on DNA extracted from paraffin-embedded samples. MSP eliminates the false positive results inherent to previous PCR-based approaches which relied on differential restriction enzyme cleavage to distinguish methylated from unmethylated DNA. (See Herman et al., 1996 and Derks et al., 2004 for more information regarding MCP.)

As used herein, the terms “administering” and “administration” refer to any method of providing a disclosed composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or compound (e.g., a composition comprising AKB-6899) together with an intended target (such as, e.g., a gene promoter, an enzyme involved in DNA methylation, a cell or population of cells, a receptor, an antigen, or other biological entity) in such a manner that the disclosed composition or compound can affect the activity of the intended target (e.g., a gene promoter, an enzyme involved in DNA methylation, receptor, transcription factor, cell, population of cells, etc.), either directly (i.e., by interacting with the target itself), or indirectly (e.g., by interacting with another gene such as an upstream gene), molecule, enzyme (e.g., a HDAC, or PHD, or DNMT), co-factor, factor, or protein on which the activity of the target is dependent). In an aspect, a disclosed composition or compound can be contacted with a cell or population of cells, such as, for example, cancer cells or tumor cells.

As used herein, the term “determining” can refer to measuring or ascertaining an activity or an event or a quantity or an amount or a change in expression and/or in activity level or in prevalence and/or incidence. For example, determining can refer to measuring or ascertaining the methylation status of one or more gene promoters. Determining can refer to measuring or ascertaining the percent methylation of one or more gene promoters. In an aspect, determining can comprise utilizing samples from a singular subject (intra-subject determination), or can comprise utilizing samples from multiple subjects (inter-subject determination). Methods of measuring or ascertaining DNA methylation are known to the art.

In an aspect, as used herein, determining can refer to measuring or ascertaining the quantity or amount of apoptosis. Determining can also refer to measuring or ascertaining the quantity or amount of activity or expression of a gene or protein of interest, such as, for example, a PHD, a DNMT, a HDAC, a HIF, etc. In an aspect, determining can also refer to measuring or ascertaining the quantity or amount of a microRNA or snoRNA. Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence. In an aspect, determining can refer to measuring the gene expression or the protein expression of one or more promoters, including APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A. In an aspect, determining can refer to measuring the gene expression or the protein expression of one or more promoters, including HIF, DNMT-1, DNMT-3a, DNMT-3b, PHD1, PHD2, PHD3, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7. In an aspect, determining can occur before an event or after an event or both before and after an event (such as an administering step).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. For example, in an aspect, an effective amount of a disclosed composition or compound is the amount effective to modulate the methylation status of one or more gene promoters. In an aspect, an effective amount of a disclosed composition is the amount effective to reduce or minimize the percent methylation of one or more gene promoters. In an aspect, an effective amount of a disclosed composition or compound is the amount effective to induce apoptosis in a desired cell or population of cells, such as, for example, cells that have aberrant DNA methylation of one or more gene promoters.

The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a disclosed composition or compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. In an aspect, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

As used herein, “interfering RNA” or “RNA interference” (RNAi) is known to the art. RNAi relies on complementarity between the RNA and its target mRNA to bring about destruction of the target. In vivo, long stretches of dsRNA can interact with the DICER endoribonuclease to be cleaved into short (21-23 nt) dsRNA with 3′ overhangs. Then, the endogenous or synthetic short stretches of dsRNA enter the multinuclease-containing RNA-induced silencing complex (RISC) and these enzymes lead to specific cleavage of complementary targets. While short (<23 nt) segments of RNA are generally considered optimal for gene silencing it has also been shown that longer (<30 nt) sequences can lead to efficient, and perhaps even more potent, gene silencing. The skilled person is familiar with the several different types of commonly used RNAi: short-interfering RNA (siRNA), short-hairpin RNA (shRNA), and micro RNA (miRNA), all of which can inhibit expression of the target gene product. The siRNA and shRNA (generally 20-22 nt in length, but they can be up to 30 nt) were designed to overcome issues with immune system stimulation and complete translation arrest observed when longer RNA sequences were used for RNAi, and to optimize the silencing effects.

“MicroRNA” or “miRNA” is an RNAi-inducing agent that refers to single-stranded, non-coding RNA molecules of about 19 to about 27 base pairs that regulate gene expression in a sequence specific manner. miRNAs are post-transcriptional regulators that bind to complementary sequences on target messenger RNA transcripts (mRNAs), usually resulting in translational repression or target degradation and gene silencing. In an aspect, the microRNAs can target methylation regulators such as the DNMTs and the HDACs. In an aspect, a disclosed composition comprising AKB-6899 can increase expression of microRNAs. In an aspect, a disclosed composition can regulate expression of microRNAs. In an aspect, microRNAs can cause differential methylation of DNMTs, HDACs, and/or other genes such as tumor suppression genes. In an aspect, microRNA sequences can be identified using polyA tail primers. In an aspect, polyA-tailed RNAs can be translated to protein.

“Short interfering RNAs” or “siRNAs”, also known as small interfering RNAs, are double-stranded RNAs that can induce sequence-specific post-transcriptional gene silencing, thereby decreasing gene expression. siRNAs can be of various lengths as long as they maintain their function. In some examples, siRNA molecules are about 19-23 nucleotides in length, such as at least 21 nucleotides, and for example at least 23 nucleotides. In one example, siRNA triggers the specific degradation of homologous RNA molecules, such as mRNAs, within the region of sequence identity between both the siRNA and the target RNA. In an example, siRNAs can cause the sequence-specific degradation of target mRNAs when base-paired with 3′ overhanging ends. The direction of dsRNA processing determines whether a sense or an antisense target RNA can be cleaved by the produced siRNA endonuclease complex. siRNAs can be generated by utilizing, for example, Invitrogen's BLOCK-IT™ RNAi Designer (rnaidesigner.invitrogen.com/rnaiexpress). Furthermore, once the nucleotide composition of siRNA molecule is determined, a publically accessible, online sequence “scrambler” can be used to ensure minimal off-target binding with human mRNA (i.e., the webpage at sirnawizard.com/scrambled.php). Furthermore, publically accessible, online sequence analysis software can be used to ensure minimal self-complementarity (i.e., the webpage at basic.northwestern.edu/biotools/oligocalc.html).

In an aspect, siRNAs can be used to modulate transcription or translation, for example, by decreasing expression of one or more disclosed genes, such as, for example, HIF, DNMT-1, DNMT-3a, DNMT-3b, PHD1, PHD2, PHD3, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7. In an aspect, siRNAs can be used to modulate transcription or translation, for example, by decreasing expression of one or more disclosed genes, such as, for example, APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A.

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e., loop) and about a 19-29 nucleotide sequence that is the reverse complement of the initial target sequence.

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter). The art is familiar with antisense oligonucleotides. Antisense oligonucleotides or molecules are designed to interact with a target nucleic acid molecule (i.e., a disclosed gene promoter) through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane or blood-brain barrier, hybridization triggered cleavage agents or intercalating agents.

In an aspect, the antisense oligonucleotide can be conjugated to another molecule, such as a peptide, hybridization triggered cross-linking agent, transport agent, or hybridization-triggered cleavage agent. Antisense oligonucleotides can include a targeting moiety that enhances uptake of the molecule by host cells. The targeting moiety can be a specific binding molecule, such as an antibody or fragment thereof that recognizes a molecule present on the surface of the host cell. Antisense molecules can be generated by utilizing the Antisense Design algorithm of Integrated DNA Technologies, Inc., available at idtdna.com/Scitools/Applications/AntiSense/Antisense.aspx/.

As used herein, “triple negative breast cancer” or “TNBC” can refer to cancer that is characterized by a lack of the estrogen receptor (ER), the progesterone receptor (PR), and the HER2/neu receptor. Standard treatment for TNBC is surgery, adjuvant chemotherapy, and radiotherapy.

As used herein, “CpG Island” refers to a genomic region of ˜1 kB that has a high G-C content, is rich in CpG dinucleotides, and is usually hypomethylated.

“Apoptosis” and methods of confirming apoptosis are known to the art and include, but are not limited to: measuring caspase-3 activity, measuring annexin V/propidium iodine binding, and measuring terminal deoxynucleotidyl transferase dUTP nick end-labeling. In an aspect, confirming apoptosis can comprise one of the following: measuring caspase-3 activity, measuring annexin V/propidium iodine binding, and measuring terminal deoxynucleotidyl transferase dUTP nick end-labeling.

“Methylation status” as used herein refers to the degree or level of methylation of a gene promoter. In an aspect, the degree or level of methylation of a gene promoter can be normal. In an aspect, the degree or level of methylation of a gene promoter can be aberrant. In an aspect, an aberrant methylation status of a gene promoter can indicate that the gene promoter is hypermethylated. In an aspect, an aberrant methylation status of a gene promoter can indicate that the gene promoter is hypomethylated. As described above, methylation status can be determined by methylation-specific PCR, which comprises two parts: (1) sodium bisulfite conversion of unmethylated cytosine's to uracil under conditions whereby methylated cytosines remains unchanged and (2) detection of the bisulfite induced sequence differences by PCR using specific primer sets for both unmethylated and methylated DNA. (See Herman et al., 1996 and Derks et al., 2004 for more information regarding MCP.)

“Percent methylation” can refer to a quantifiable amount of methylation of a gene promoter. The quantifiable amount can be obtained by measuring the amount of methylation of a gene promoter in an affected tissue and comparing that to the amount of methylation of the same gene promoter in an unaffected tissue. In an aspect, the quantifiable amount can be obtained by measuring the amount of methylation of a gene promoter in a subject (e.g., a subject with cancer or a subject diagnosed with or suspected of having a disease or disorder characterized by hypermethylation of one or more gene promoters) and comparing that to the amount of methylation of the same gene promoter in one or more other subjects (e.g., one or more other subjects that do not have cancer or subjects that have not been diagnosed with or that are not suspected of having a disease or disorder characterized by hypermethylation of one or more gene promoters).

### B. COMPOSITIONS

Disclosed herein is a composition for modulating DNA methylation of one or more gene promoters, comprising an effective amount of a compound of the formula:

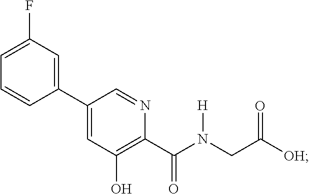 [SMILES: O=C(O)CNC(=O)c1ncc(-c2cccc(F)c2)cc1O]

or a pharmaceutically acceptable salt thereof.

Disclosed herein is a composition for treating a subject diagnosed with or suspected of having a disease or disorder characterized by DNA hypermethylation, comprising an effective amount of a compound of the formula:

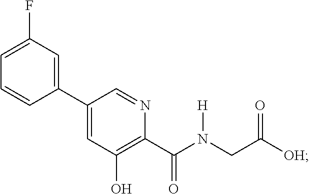 [SMILES: O=C(O)CNC(=O)c1ncc(-c2cccc(F)c2)cc1O]

or a pharmaceutically acceptable salt thereof. In an aspect, a disclosed composition for treating a subject diagnosed with or suspected of having a disease or disorder characterized by DNA hypermethylation can comprise one or more anti-cancer agents or one or more chemotherapeutic agents.

Disclosed herein is a composition for decreasing c-Myc expression, comprising an effective amount of a compound of the formula:

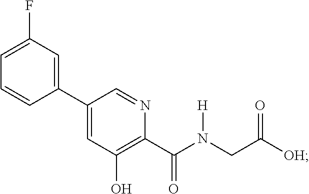 [SMILES: O=C(O)CNC(=O)c1ncc(-c2cccc(F)c2)cc1O]

or a pharmaceutically acceptable salt thereof.

Disclosed herein is a composition for increasing desmoplakin expression, comprising an effective amount of a compound of the formula:

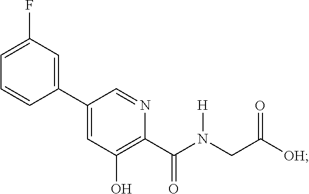 [SMILES: O=C(O)CNC(=O)c1ncc(-c2cccc(F)c2)cc1O]

or a pharmaceutically acceptable salt thereof.

Disclosed herein is a composition for treating cancer, comprising an effective amount of a compound of the formula:

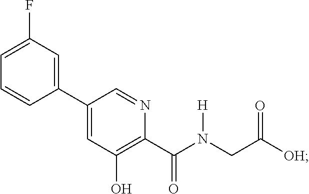 [SMILES: O=C(O)CNC(=O)c1ncc(-c2cccc(F)c2)cc1O]

or a pharmaceutically acceptable salt thereof. In an aspect, a disclosed composition for inhibiting metises can comprise one or more anti-cancer agents or one or more chemotherapeutic agents.

Disclosed herein is a composition for treating cancer, comprising an effective amount of a compound of the formula:

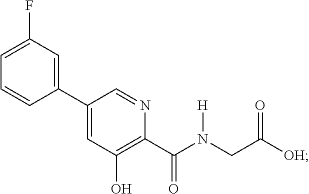 [SMILES: O=C(O)CNC(=O)c1ncc(-c2cccc(F)c2)cc1O]

or a pharmaceutically acceptable salt thereof; and one or more chemotherapeutic agents.

Disclosed herein is a composition for treating cancer, comprising an effective amount of a compound of the formula:

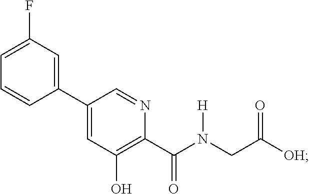 [SMILES: O=C(O)CNC(=O)c1ncc(-c2cccc(F)c2)cc1O]

a pharmaceutically acceptable salt thereof; and one or more anti-cancer agents.

Disclosed herein are pharmaceutical compositions comprising a disclosed composition comprising an effective amount of a compound of the formula:

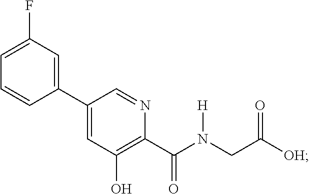 [SMILES: O=C(O)CNC(=O)c1ncc(-c2cccc(F)c2)cc1O]

or a pharmaceutically acceptable salt thereof.

In an aspect, a disclosed composition can inhibit the expression of one or more DNA methyltransferases. DNA methyltransferases (DNA MTs), including non-mammalian homologs, are known to the art. In an aspect, a DNA methyltransferase can comprise a human DNA methyltransferase. In an aspect, a DNA methyltransferase can comprise DNMT-1, DNMT-3A, or DNMT-3B. In an aspect, a disclosed composition can inhibit the expression of DNMT-1. In an aspect, a disclosed composition can inhibit the expression of DNMT-3A. In an aspect, a disclosed composition can inhibit the expression of DNMT-3B. In an aspect, a disclosed composition can inhibit the expression of DNMT-1, DNMT-3A, and DNMT-3B.

In an aspect, a disclosed composition can inhibit the expression of one or more histone deacetylases. Histone deacetylases, including non-mammalian homologs, are known to the art. In an aspect, a histone deacetylases can comprise a human histone deacetylase. In an aspect, a histone deacetylase can comprise any known HDAC, such as, for example, HDAC1, HDAC2, HDAC3, HDAC4, HDAC5, HDAC6, HDAC7, HDAC8, HDAC9, HDAC10, or HDAC11. In an aspect, a histone deacetylase can comprise SIRT1, SIRT2, SIRT3, SIRT4, SIRT5, SIRT6, or SIRT7. In an aspect, a disclosed composition can inhibit the expression of one or more of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of a combination of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of each of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of one or more of SIRT1, SIRT2, SIRT3, SIRT4, SIRT5, SIRT6, and SIRT7. In an aspect, a disclosed composition can inhibit the expression of a combination of SIRT1, SIRT2, SIRT3, SIRT4, SIRT5, SIRT6, and SIRT7. In an aspect, a disclosed composition can inhibit the expression of one or more HDACs and can inhibit the expression of one or more SIRTs.

In an aspect, disclosed composition can inhibit the expression of one or more prolyl hydroxylases. Prolyl hydroxylases, including non-mammalian homologs, are known to the art. In an aspect, a prolyl hydroxylase can comprise a human prolyl hydroxylase. In an aspect, a prolyl hydroxylases can comprise any known prolyl hydroxylase, such as, for example, PHD1, PHD2, and PHD3. In an aspect, a disclosed composition can inhibit the expression of one or more prolyl hydroxylases. In an aspect, a disclosed composition can inhibit the expression of PHD1. In an aspect, a disclosed composition can inhibit the expression of PHD2. In an aspect, a disclosed composition can inhibit the expression of PHD3. In an aspect, a disclosed composition can inhibit the expression of a combination of PHD1, PHD2, and PHD3. In an aspect, a disclosed composition can inhibit the expression of each of PHD1, PHD2, and PHD3.

In an aspect, a disclosed composition can inhibit one or more of the disclosed DNMTs, HDACs, SIRTs, or PHDs in a disclosed method.

Disclosed herein are pharmaceutical compositions comprising the disclosed compositions and compounds (including pharmaceutically acceptable salt(s) thereof) as an active ingredient, a pharmaceutically acceptable carrier, and, optionally, other therapeutic ingredients or adjuvants. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microciystalline cellulose, diluents, granulating agents, lubricants, binders, disintegrating agents, and the like can be used to form oral solid preparations such as powders, capsules and tablets. Tablets and capsules are the preferred oral dosage units whereby solid pharmaceutical carriers are employed. Optionally, tablets can be coated by standard aqueous or nonaqueous techniques. A tablet containing a composition or compound disclosed herein can be prepared by compression or molding, optionally with one or more accessory ingredients or adjuvants. Compressed tablets can be prepared by compressing, in a suitable machine, a disclosed composition or disclosed compound in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

It is understood that the disclosed compositions can be prepared from the disclosed compounds. It is also understood that the disclosed compositions can be employed in the disclosed methods of using.

### C. METHODS

### i) Method of Modulating DNA Methylation

Disclosed herein is a method of modulating DNA methylation of one or more gene promoters in a subject, comprising administering to a subject an effective amount of composition comprising a compound of the formula:

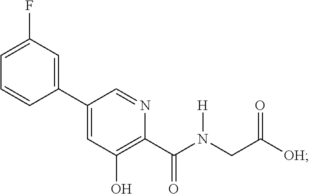 [SMILES: O=C(O)CNC(=O)c1ncc(-c2cccc(F)c2)cc1O]

or a pharmaceutically acceptable salt thereof. In an aspect, a disclosed method of modulating DNA methylation can comprise determining the methylation status of the one or more gene promoters.

Disclosed herein is a method of modulating DNA methylation of one or more gene promoters in a subject, comprising: identifying a subject in need of treatment by determining the methylation status of one or more gene promoters; and administering to a subject an effective amount of composition comprising a compound of the formula:

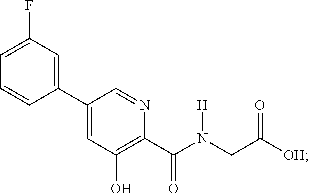 [SMILES: O=C(O)CNC(=O)c1ncc(-c2cccc(F)c2)cc1O]

or a pharmaceutically acceptable salt thereof.

Methods of measuring or ascertaining the methylation status of one or more gene promoters are known to the art. For example, methylation specific PCR, which is known to the art, can be used. In an aspect, determining the methylation status of the one or more gene promoters can comprise comparing the methylation status of the one or more gene promoters in an affected tissue of the subject to the methylation status of the one or more gene promoters in an unaffected tissue of the subject. In an aspect, affected tissue can be a tumor or a cancer and unaffected tissue can be something other than a tumor or a cancer. In an aspect, determining the methylation status of the one or more gene promoters can comprise measuring the percent methylation of the one or more promoters.

In an aspect of a disclosed method of modulating DNA methylation, a subject can be an unhealthy subject. In an aspect, an unhealthy subject can be a subject afflicted with or suffering from a disease, a condition, a disorder, or an illness.

In an aspect of a disclosed method of modulating DNA methylation, prior to the administering step, the one or more gene promoters are hypermethylated. In other words, the one or more gene promoters of interest have a higher level of percent methylation. The level of percent methylation can be compared between two subjects, such as, for example, a subject diagnosed with or suspected of having a specific disease or disorder and a subject not diagnosed with or not suspected of having a specific disease or disorder. The level of percent methylation can be compared between within a subject, such as, for example, between an affected tissue or organ or cell and an unaffected tissue or organ or cell. In an aspect, an affected tissue or organ or cell can be a cancerous or tumorous tissue or organ or cell.

In an aspect of a disclosed method of modulating DNA methylation, the method can comprise identifying a subject in need thereof prior to the administering step.

In an aspect of a disclosed method of modulating DNA methylation, after the administering step, there can be a change in the methylation status of the one or more gene promoters. For example, in an aspect, a change in methylation status can comprise a decrease in the percent methylation of the one or more gene promoters. If, after the administering step, the desired methylation status is not achieved, then the method can comprise repeating the administration of an effective amount the composition. In an aspect, the desired methylation status can be a decrease in the percent methylation of the one or more gene promoters. In an aspect, the administering step can be repeated prior to and after the methylation status is determined. In an aspect, the administering step can be repeated after the methylation status is determined. In an aspect, the administering step can be repeated one or more times, such as, for example, two, three, four, five, ten, fifteen, twenty, thirty, forty, fifty, or more times. In an aspect, the administering step can occur hourly, every 3 hours, every 6 hours, every 12 hours, every 18 hours, daily, weekly, bi-weekly, monthly, bi-monthly, yearly, bi-annually, every 5 years, or every 10 years of a subject's life. In an aspect, a disclosed method can comprise determining the methylation status of the one or more gene promoters after a singular administering step. In an aspect, a disclosed method of modulating DNA methylation can comprise determining the methylation status of the one or more gene promoters after some administering steps. In an aspect, a disclosed method can comprise determining the methylation status of the one or more gene promoters after every administering step.

In an aspect of a disclosed method of modulating DNA methylation, a disclosed composition comprising a compound of the formula:

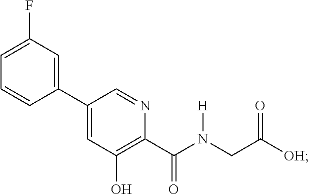 [SMILES: O=C(O)CNC(=O)c1ncc(-c2cccc(F)c2)cc1O]

or a pharmaceutically acceptable salt thereof can inhibit the expression of one or more DNA methyltransferases. DNA methyltransferases (DNA MTs), including non-mammalian homologs, are known to the art. In an aspect, a DNA methyltransferase can comprise a human DNA methyltransferase. In an aspect, a DNA methyltransferase can comprise DNMT-1, DNMT-3A, or DNMT-3B. In an aspect, a disclosed composition can inhibit the expression of DNMT-1. In an aspect, a disclosed composition can inhibit the expression of DNMT-3A. In an aspect, a disclosed composition can inhibit the expression of DNMT-3B. In an aspect, a disclosed composition can inhibit the expression of DNMT-1, DNMT-3A, and DNMT-3B.

In an aspect of a disclosed method of modulating DNA methylation, a disclosed composition comprising a compound of the formula:

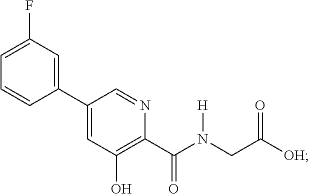 [SMILES: O=C(O)CNC(=O)c1ncc(-c2cccc(F)c2)cc1O]

or a pharmaceutically acceptable salt thereof can inhibit the expression of one or more histone deacetylases. Histone deacetylases, including non-mammalian homologs, are known to the art. In an aspect, a histone deacetylases can comprise a human histone deacetylase. In an aspect, a histone deacetylase can comprise any known HDAC, such as, for example, HDAC1, HDAC2, HDAC3, HDAC4, HDAC5, HDAC6, HDAC7, HDAC8, HDAC9, HDAC10, or HDAC11. In an aspect, a histone deacetylase can comprise SIRT1, SIRT2, SIRT3, SIRT4, SIRT5, SIRT6, or SIRT7. In an aspect, a disclosed composition can inhibit the expression of one or more of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of a combination of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of each of HDAC1, HDAC2, HDAC3, HDAC4, HDAC5, HDAC6, HDAC7, HDAC8, HDAC9, HDAC10, and HDAC11 In an aspect, a disclosed composition can inhibit the expression of one or more of SIRT1, SIRT2, SIRT3, SIRT4, SIRT5, SIRT6, and SIRT7. In an aspect, a disclosed composition can inhibit the expression of a combination of SIRT1, SIRT2, SIRT3, SIRT4, SIRT5, SIRT6, and SIRT7. In an aspect, a disclosed composition can inhibit the expression of one or more HDACs and can inhibit the expression of one or more SIRTs.

In an aspect of a disclosed method of modulating DNA methylation, a disclosed composition comprising a compound of the formula:

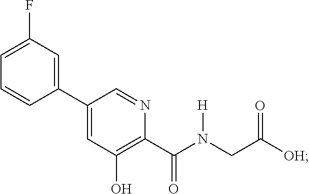 [SMILES: O=C(O)CNC(=O)c1ncc(-c2cccc(F)c2)cc1O]

or a pharmaceutically acceptable salt thereof can inhibit the expression of one or more prolyl hydroxylases. Prolyl hydroxylases, including non-mammalian homologs, are known to the art. In an aspect, a prolyl hydroxylase can comprise a human prolyl hydroxylase. In an aspect, a prolyl hydroxylases can comprise any known prolyl hydroxylase, such as, for example, PHD1, PHD2, and PHD3. In an aspect, a disclosed composition can inhibit the expression and/or activity of one or more prolyl hydroxylases. In an aspect, a disclosed composition can inhibit the expression and/or activity of PHD1. In an aspect, a disclosed composition can inhibit the expression and/or activity of PHD2. In an aspect, a disclosed composition can inhibit the expression and/or activity of PHD3. In an aspect, a disclosed composition can inhibit the expression and/or activity of a combination of PHD1, PHD2, and PHD3. In an aspect, a disclosed composition can inhibit the expression and/or activity of each of PHD1, PHD2, and PHD3.

An administering step of a disclosed method of modulating DNA methylation can comprise any route of administration known to the art. For example, in an aspect, an administering step can comprise intraperitoneal administration. For example, in an aspect, an administering step can comprise oral administration. For example, in an aspect, an administering step can comprise intravenous administration.

In a disclosed method of modulating DNA methylation, a gene promoter can comprise a desmoplakin (DSP) gene promoter, a c-myc gene promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a cytochrome P450, family 1, subfamily B, polypeptide 1 (CYP1B1) promoter, a deleted in liver cancer 1 (DLC1) promoter, an E-cadherin (CDH1) promoter, a fragile histidine triad (FHIT) promoter, a H-cadherin (CDH13) promoter, an O-6-methylguanine-DNA methyltransferase (MGMT) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, an Adenomatous polyposis coli (APC) promoter, an Amyloid beta A4 precursor protein-binding family A member 1 (APBA1) promoter, a cell adhesion molecule 1 (CADM1) promoter, a chemokine (C—X—C motif) ligand 12 (CXCL12) promoter, a methylenetetrahydrofolate reductase (NAD(P)H) (MTHFR) promoter, a mutL homolog 1 promoter, nonpolyposis type 2 (MLH1) promoter, a Ras association (RalGDS/AF-6) domain family member 2 (RASSF2) promoter, a secreted frizzled-related protein 1 (SFRP1) promoter, a transcription factor 21 (TCF21) promoter, or a vascular endothelial growth factor-A (VEGF-A) promoter.

A disclosed method of modulating DNA methylation can comprise one or more of the following gene promoters: DSP, C-MYC, CDKN1C, CDKN2A, CDKN2B, CYP1B1, DLC1, CDH1, FHIT, CDH13, MGMT, OPCML, PAX5, PRDM2, RASSF1, APC, APBA1, CADM1, CXCL12, MTHFR, MLH1, RASSF2, SFRP1, TCF21, and VEGF-A.

In an aspect, a disclosed method of modulating DNA methylation can comprise one or more gene promoters of one or more tumor suppressor genes (TSGs) as known to the art.

In a disclosed method of modulating DNA methylation, a gene promoter can comprise an estrogen receptor gene (ER) promoter, a breast cancer 1 gene (BRCA1) promoter, an epithelial cadherin gene (E-cad) promoter, a TMS1 gene promoter, an insulin-like growth factor binding protein 7 gene (IGFBP7) promoter, a p16 promoter, a retinoic acid receptor gene (RARβ2) promoter, or a Ras association (RalGDS/AF-6) domain family member 1 gene (RASSF1A) promoter.

A disclosed method of modulating DNA methylation can comprise one or more of the following gene promoters: ER, BRCA1, E-cad, TMS1, IGFBP7, RARβ2, and RASSF1A.

A disclosed method of modulating DNA methylation can comprise one or more of the following gene promoters: APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A.

In a disclosed method of modulating DNA methylation, a subject can have a disease or disorder that is not vascular leak. In a disclosed method of modulating DNA methylation, a subject can have a disease or disorder that is not retinopathy. In a disclosed method of modulating DNA methylation, a subject can have a disease or disorder that is not critical limb ischemia (CLI).

In a disclosed method of modulating DNA methylation, a subject can have cancer. In an aspect, the cancer can be a cancer that is caused by the hypermethylation of one or more gene promoters. For example, the cancer can be any cancer identified in Table 1. In an aspect, a disclosed method can comprise administering to a subject one or more anti-cancer agents. In an aspect, a subject can have breast cancer and the one or more gene promoters can comprise a desmoplakin (DSP) promoter. In an aspect, breast cancer can be triple negative breast cancer. In an aspect, a subject can have melanoma and the one or more gene promoters can comprise a desmoplakin (DSP) promoter. In an aspect of a disclosed method, a subject can have cervical cancer and the one or more gene promoters can comprise a desmoplakin (DSP) promoter or a c-Myc promoter. In an aspect, a subject can have lung cancer and the one or more gene promoters can comprise a desmoplakin (DSP) promoter, an Adenomatous polyposis coli (APC) promoter, an Amyloid beta A4 precursor protein-binding family A member 1 (APBA1) promoter, a cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

In an aspect, a disclosed method of modulating DNA methylation can comprise ameliorating one or more symptoms associated with aberrant DNA methylation, such as, for example, hypermethylation, of one or more gene promoters.

ii) Method of Treating a Subject Diagnosed with or Suspected of Having a Disease or Disorder Characterized by DNA Hypermethylation

Disclosed herein is a method of treating a subject diagnosed with or suspected of having a disease or disorder characterized by DNA hypermethylation, comprising administering to a subject an effective amount of a composition comprising a compound of the formula:

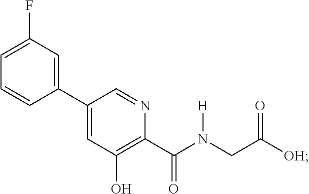 [SMILES: O=C(O)CNC(=O)c1ncc(-c2cccc(F)c2)cc1O]

or a pharmaceutically acceptable salt thereof.

In an aspect, a disclosed method of treating a subject diagnosed with or suspected of having a disease or disorder characterized by DNA hypermethylation can comprise determining the methylation status of the one or more gene promoters. Methods of measuring or ascertaining the methylation status of one or more gene promoters are known to the art and discussed above. In an aspect, determining the methylation status of the one or more gene promoters can comprise comparing the methylation status of the one or more gene promoters in an affected tissue of the subject to the methylation status of the one or more gene promoters in an unaffected tissue of the subject. In an aspect, affected tissue can be a tumor or a cancer and unaffected tissue can be something other than a tumor or a cancer (i.e., a non-cancerous tissue or cell or sample). In an aspect, determining the methylation status of the one or more gene promoters can comprise measuring the percent methylation of the one or more promoters.

In an aspect of a method of treating a subject diagnosed with or suspected of having a disease or disorder characterized by DNA hypermethylation, a subject can be an unhealthy subject. In an aspect, an unhealthy subject can be a subject afflicted with or suffering from a disease, a condition, a disorder, or an illness, such as, for example, one that is characterized by DNA hypermethylation.

In an aspect of a disclosed method of treating a subject diagnosed with or suspected of having a disease or disorder characterized by DNA hypermethylation, prior to the administering step, the one or more gene promoters are hypermethylated. In other words, the one or more gene promoters of interest have a higher level of percent methylation. The level of percent methylation can be compared between two subjects, such as, for example, a subject diagnosed with or suspected of having a specific disease or disorder characterized by DNA hypermethylation and a subject not diagnosed with or not suspected of having a specific disease or disorder characterized by DNA hypermethylation. The level of percent methylation can be compared between within a subject, such as, for example, between an affected tissue or organ or cell and an unaffected tissue or organ or cell. In an aspect, an affected tissue or organ or cell can be a cancerous or tumorous tissue or organ or cell.

In an aspect of a disclosed method of treating a subject diagnosed with or suspected of having a disease or disorder characterized by DNA hypermethylation, the method can comprise identifying a subject in need thereof prior to the administering step.

In an aspect, a disease that can be regulated by DNA hypermethylation can be as follows: Beckwith-Wiedemann syndrome (i.e., hypermethylation of CDKN1C and/or H19 genes); Prader-Willi syndrome (i.e., hypermethylation of MKRN3, MAGEL2, NDN, SNURF/SNRPN, and/or IPW genes); Angelman syndrome (i.e., hypermethylation of UBE3A and/or ATPC10C genes); Fragile X syndrome (i.e., hypermethylation of FMR1 gene); Myotonic dystrophy (i.e., hypermethylation of DMPK, SIX5, and/or other genes); ATRX syndrome (i.e., hypermethylation of ATRX gene); development (i.e., hypermethylation of one or more genes regulating development); sepsis (i.e., hypermethylation of DNMT-1, DNMT-3A, and/or DNMT-3B genes); and one or more genes involved in the aging process.

In an aspect of a disclosed method of treating a subject diagnosed with or suspected of having a disease or disorder characterized by DNA hypermethylation, after the administering step, there can be a change in the methylation status of the one or more gene promoters. For example, in an aspect, a change in methylation status can comprise a decrease in the percent methylation of the one or more gene promoters. If, after the administering step, the desired methylation status is not achieved, then the method can comprise repeating the administration of an effective amount the composition. In an aspect, the desired methylation status can be a decrease in the percent methylation of the one or more gene promoters. In an aspect, the administering step can be repeated prior to and after the methylation status is determined. In an aspect, the administering step can be repeated after the methylation status is determined. In an aspect, the administering step can be repeated one or more times, such as, for example, two, three, four, five, ten, fifteen, twenty, thirty, forty, fifty, or more times. In an aspect, the administering step can occur hourly, every 3 hours, every 6 hours, every 12 hours, every 18 hours, daily, weekly, bi-weekly, monthly, bi-monthly, yearly, bi-annually, every 5 years, or every 10 years of a subject's life. In an aspect, a disclosed method can comprise determining the methylation status of the one or more gene promoters after a singular administering step. In an aspect, a disclosed method can comprise determining the methylation status of the one or more gene promoters after some administering steps. In an aspect, a disclosed method can comprise determining the methylation status of the one or more gene promoters after every administering step.

In an aspect of a disclosed method of treating a subject diagnosed with or suspected of having a disease or disorder characterized by DNA hypermethylation, a disclosed composition comprising a compound of the formula:

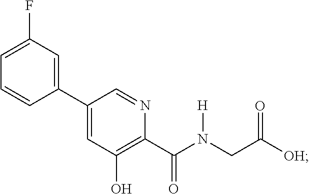 [SMILES: O=C(O)CNC(=O)c1ncc(-c2cccc(F)c2)cc1O]

or a pharmaceutically acceptable salt thereof can inhibit the expression of one or more DNA methyltransferases. DNA methyltransferases are known to the art and discussed above. In an aspect, a DNA methyltransferase can comprise a human DNA methyltransferase. In an aspect, a DNA methyltransferase can comprise DNMT-1, DNMT-3A, or DNMT-3B. In an aspect, a disclosed composition can inhibit the expression of DNMT-1. In an aspect, a disclosed composition can inhibit the expression of DNMT-3A. In an aspect, a disclosed composition can inhibit the expression of DNMT-3B. In an aspect, a disclosed composition can inhibit the expression of DNMT-1, DNMT-3A, and DNMT-3B.

In an aspect of a disclosed method of treating a subject diagnosed with or suspected of having a disease or disorder characterized by DNA hypermethylation, a disclosed composition comprising a compound of the formula:

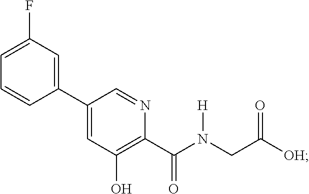 [SMILES: O=C(O)CNC(=O)c1ncc(-c2cccc(F)c2)cc1O]

or a pharmaceutically acceptable salt thereof can inhibit the expression of one or more histone deacetylases. Histone deacetylases are known to the art and are discussed above. In an aspect, a histone deacetylases can comprise a human histone deacetylase. In an aspect, a histone deacetylase can comprise any known HDAC, such as, for example, HDAC1, HDAC2, HDAC3, HDAC4, HDAC5, HDAC6, HDAC7, HDAC8, HDAC9, HDAC10, or HDAC11. In an aspect, a histone deacetylase can comprise SIRT1, SIRT2, SIRT3, SIRT4, SIRT5, SIRT6, or SIRT7. In an aspect, a disclosed composition can inhibit the expression of one or more of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of a combination of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of each of HDAC1, HDAC2, HDAC3, HDAC4, HDAC5, HDAC6, HDAC7, HDAC8, HDAC9, HDAC10, and HDAC11 In an aspect, a disclosed composition can inhibit the expression of one or more of SIRT1, SIRT2, SIRT3, SIRT4, SIRT5, SIRT6, and SIRT7. In an aspect, a disclosed composition can inhibit the expression of a combination of SIRT1, SIRT2, SIRT3, SIRT4, SIRT5, SIRT6, and SIRT7. In an aspect, a disclosed composition can inhibit the expression of one or more HDACs and can inhibit the expression of one or more SIRTs.

In an aspect of a disclosed method of treating a subject diagnosed with or suspected of having a disease or disorder characterized by DNA hypermethylation, a disclosed composition comprising a compound of the formula:

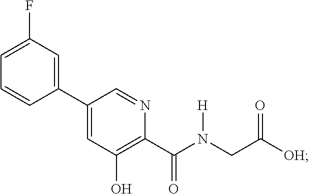 [SMILES: O=C(O)CNC(=O)c1ncc(-c2cccc(F)c2)cc1O]

or a pharmaceutically acceptable salt thereof can inhibit the expression of one or more prolyl hydroxylases. Prolyl hydroxylases, including non-mammalian homologs, are known to the art and are discussed above. In an aspect, a prolyl hydroxylase can comprise a human prolyl hydroxylase. In an aspect, a prolyl hydroxylases can comprise any known prolyl hydroxylase, such as, for example, PHD1, PHD2, and PHD3. In an aspect, a disclosed composition can inhibit the expression and/or activity of one or more prolyl hydroxylases. In an aspect, a disclosed composition can inhibit the expression and/or activity of PHD1. In an aspect, a disclosed composition can inhibit the expression and/or activity of PHD2. In an aspect, a disclosed composition can inhibit the expression and/or activity of PHD3. In an aspect, a disclosed composition can inhibit the expression and/or activity of a combination of PHD1, PHD2, and PHD3. In an aspect, a disclosed composition can inhibit the expression and/or activity of each of PHD1, PHD2, and PHD3.

An administering step of a disclosed method of treating a subject diagnosed with or suspected of having a disease or disorder characterized by DNA hypermethylation can comprise any route of administration known to the art. For example, in an aspect, an administering step can comprise intraperitoneal administration. For example, in an aspect, an administering step can comprise oral administration. For example, in an aspect, an administering step can comprise intravenous administration. In an aspect, a subject does not have vascular leak, retinopathy, or critical limb ischemia (CLI).

In a disclosed method of treating a subject diagnosed with or suspected of having a disease or disorder characterized by DNA hypermethylation, a gene promoter can comprise a desmoplakin (DSP) gene promoter, a c-myc gene promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a cytochrome P450, family 1, subfamily B, polypeptide 1 (CYP1B1) promoter, a deleted in liver cancer 1 (DLC1) promoter, an E-cadherin (CDH1) promoter, a fragile histidine triad (FHIT) promoter, a H-cadherin (CDH13) promoter, an O-6-methylguanine-DNA methyltransferase (MGMT) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, an Adenomatous polyposis coli (APC) promoter, an Amyloid beta A4 precursor protein-binding family A member 1 (APBA1) promoter, a cell adhesion molecule 1 (CADM1) promoter, a chemokine (C—X—C motif) ligand 12 (CXCL12) promoter, a methylenetetrahydrofolate reductase (NAD(P)H) (MTHFR) promoter, a mutL homolog 1 promoter, nonpolyposis type 2 (MLH1) promoter, a Ras association (RalGDS/AF-6) domain family member 2 (RASSF2) promoter, a secreted frizzled-related protein 1 (SFRP1) promoter, a transcription factor 21 (TCF21) promoter, or a vascular endothelial growth factor-A (VEGF-A) promoter.

In an aspect, a disclosed method of treating a subject diagnosed with or suspected of having a disease or disorder characterized by DNA hypermethylation can comprise one or more of the following gene promoters: DSP, C-MYC, CDKN1C, CDKN2A, CDKN2B, CYP1B1, DLC1, CDH1, FHIT, CDH13, MGMT, OPCML, PAX5, PRDM2, RASSF1, APC, APBA1, CADM1, CXCL12, MTHFR, MLH1, RASSF2, SFRP1, TCF21, and VEGF-A.

In an aspect, a disclosed method of treating a subject diagnosed with or suspected of having a disease or disorder characterized by DNA hypermethylation can comprise one or more gene promoters of one or more tumor suppressor genes (TSGs) as known to the art.

In a disclosed method of treating a subject diagnosed with or suspected of having a disease or disorder characterized by DNA hypermethylation, a gene promoter can comprise an estrogen receptor gene (ER) promoter, a breast cancer 1 gene (BRCA1) promoter, an epithelial cadherin gene (E-cad) promoter, a TMS1 gene promoter, an insulin-like growth factor binding protein 7 gene (IGFBP7) promoter, a p16 promoter, a retinoic acid receptor gene (RARβ2) promoter, or a Ras association (RalGDS/AF-6) domain family member 1 gene (RASSF1A) promoter.

In an aspect, a disclosed method of treating a subject diagnosed with or suspected of having a disease or disorder characterized by DNA hypermethylation can comprise one or more of the following gene promoters: ER, BRCA1, E-cad, TMS1, IGFBP7, RARβ2, and RASSF1A.

In an aspect, a disclosed method of treating a subject diagnosed with or suspected of having a disease or disorder characterized by DNA hypermethylation can comprise one or more of the following gene promoters: APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A.

In a disclosed method of treating a subject diagnosed with or suspected of having a disease or disorder characterized by DNA hypermethylation, the disease or disorder characterized by DNA hypermethylation can be something other than cancer.

In a disclosed method of treating a subject diagnosed with or suspected of having a disease or disorder characterized by DNA hypermethylation, a subject can have cancer. In an aspect, the cancer can be a cancer that is caused by the hypermethylation of one or more gene promoters. For example, the cancer can be any cancer identified in Table 1. In an aspect, a disclosed method can comprise administering to a subject one or more anti-cancer agents. In an aspect, a subject can have breast cancer and the one or more gene promoters can comprise a desmoplakin (DSP) promoter. In an aspect, breast cancer can be triple negative breast cancer. In an aspect, a subject can have melanoma and the one or more gene promoters can comprise a desmoplakin (DSP) promoter. In an aspect, a subject can have cervical cancer and the one or more gene promoters can comprise a desmoplakin (DSP) promoter or a c-Myc promoter. In an aspect, a subject can have lung cancer and the one or more gene promoters can comprise a desmoplakin (DSP) promoter, an Adenomatous polyposis coli (APC) promoter, an Amyloid beta A4 precursor protein-binding family A member 1 (APBA1) promoter, a cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

In an aspect, a disclosed method of treating a subject diagnosed with or suspected of having a disease or disorder characterized by DNA hypermethylation can comprise ameliorating one or more symptoms associated with DNA hypermethylation of one or more gene promoters,

iii) Method of Decreasing C-MYC Expression

Disclosed herein is a method of decreasing c-myc expression in a subject, comprising administering to a subject an effective amount of a composition comprising a compound of the formula:

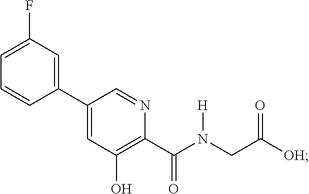 [SMILES: O=C(O)CNC(=O)c1ncc(-c2cccc(F)c2)cc1O]

or a pharmaceutically acceptable salt thereof. In an aspect, a disclosed method of decreasing c-myc expression can comprise altering the methylation status of the c-myc promoter. In an aspect, a disclosed method of decreasing c-myc expression can comprise determining the methylation status of the c-myc promoter. Methods of measuring or ascertaining the methylation status of a promoter such as the c-myc promoter are known to the art and are discussed above. In an aspect, determining the methylation status of the c-myc promoter can comprise comparing the methylation status of the one or more gene promoters in an affected tissue of the subject to the methylation status of the one or more gene promoters in an unaffected tissue of the subject. In an aspect, affected tissue can be a tumor or a cancer and unaffected tissue can be something other than a tumor or a cancer (i.e., a non-cancerous tissue or cell or sample). In an aspect, determining the methylation status of the c-myc promoter can comprise measuring the percent methylation of the c-myc promoter.

In an aspect of a method of decreasing c-myc expression in a subject, a subject can be an unhealthy subject. In an aspect, an unhealthy subject can be a subject afflicted with or suffering from a disease, a condition, a disorder, or an illness.

In a disclosed method of decreasing c-myc expression, a subject can have a disease or disorder that is not vascular leak. In a disclosed method of decreasing c-myc expression, a subject can have a disease or disorder that is not retinopathy. In a disclosed method of decreasing c-myc expression, a subject can have a disease or disorder that is not critical limb ischemia (CLI).

In an aspect of a disclosed method of decreasing c-myc expression in a subject, the method can comprise identifying a subject in need thereof prior to the administering step.

In an aspect of a disclosed method of decreasing c-myc expression, prior to the administering step, the c-myc promoter is hypermethylated. In other words, the c-myc promoter has a higher level of percent methylation. The level of percent methylation can be compared between two subjects, such as, for example, a subject diagnosed with or suspected of having a specific disease or disorder and a subject not diagnosed with or not suspected of having a specific disease or disorder. The level of percent methylation can be compared between within a subject, such as, for example, between an affected tissue or organ or cell and an unaffected tissue or organ or cell. In an aspect, an affected tissue or organ or cell can be a cancerous or tumorous tissue or organ or cell.

In an aspect of a disclosed method of decreasing c-myc expression, after the administering step, there can be a change in the methylation status of the c-myc promoter. For example, in an aspect, a change in methylation status can comprise a decrease in the percent methylation of the c-myc promoter. If, after the administering step, the desired methylation status of the c-myc promoter is not achieved, then the method can comprise repeating the administration of an effective amount the composition. In an aspect, the desired methylation status can be a decrease in the percent methylation of the c-myc promoter. In an aspect, the administering step can be repeated prior to and after the methylation status is determined. In an aspect, the administering step can be repeated after the methylation status is determined. In an aspect, the administering step can be repeated one or more times, such as, for example, two, three, four, five, ten, fifteen, twenty, thirty, forty, fifty, or more times. In an aspect, the administering step can occur hourly, every 3 hours, every 6 hours, every 12 hours, every 18 hours, daily, weekly, bi-weekly, monthly, bi-monthly, yearly, bi-annually, every 5 years, or every 10 years of a subject's life. In an aspect, a disclosed method can comprise determining the methylation status of the c-myc promoter after a singular administering step. In an aspect, a disclosed method of modulating DNA methylation can comprise determining the methylation status of the c-myc promoter after some administering steps. In an aspect, a disclosed method can comprise determining the methylation status of the c-myc promoter after every administering step.

In a disclosed method of decreasing c-myc expression, a subject can have cancer. In an aspect, the cancer can be a cancer that is caused by the hypermethylation of one or more gene promoters. For example, the cancer can be any cancer identified in Table 1. In an aspect, a decrease in c-myc expression can inhibit metastases in the subject. In an aspect, a disclosed method of decreasing c-myc expression can comprise administering to a subject one or more anti-cancer agents.

In an aspect of a disclosed method of decreasing c-myc expression, a disclosed composition comprising a compound of the formula:

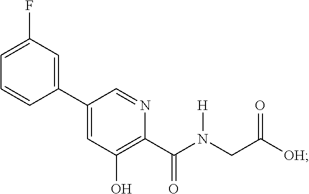 [SMILES: O=C(O)CNC(=O)c1ncc(-c2cccc(F)c2)cc1O]

or a pharmaceutically acceptable salt thereof can inhibit the expression of one or more DNA methyltransferases. DNA methyltransferases (DNA MTs), including non-mammalian homologs, are known to the art. In an aspect, a DNA methyltransferase can comprise a human DNA methyltransferase. In an aspect, a DNA methyltransferase can comprise DNMT-1, DNMT-3A, or DNMT-3B. In an aspect, a disclosed composition can inhibit the expression of DNMT-1. In an aspect, a disclosed composition can inhibit the expression of DNMT-3A. In an aspect, a disclosed composition can inhibit the expression of DNMT-3B. In an aspect, a disclosed composition can inhibit the expression of DNMT-1, DNMT-3A, and DNMT-3B.

In an aspect of a disclosed method of decreasing c-myc expression, a disclosed composition comprising a compound of the formula:

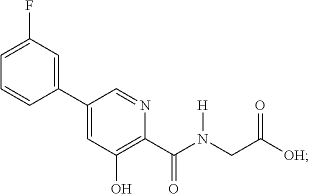 [SMILES: O=C(O)CNC(=O)c1ncc(-c2cccc(F)c2)cc1O]

or a pharmaceutically acceptable salt thereof can inhibit the expression of one or more histone deacetylases. Histone deacetylases, including non-mammalian homologs, are known to the art. In an aspect, a histone deacetylases can comprise a human histone deacetylase. In an aspect, a histone deacetylase can comprise any known HDAC, such as, for example, HDAC1, HDAC2, HDAC3, HDAC4, HDAC5, HDAC6, HDAC7, HDAC8, HDAC9, HDAC10, or HDAC11. In an aspect, a histone deacetylase can comprise SIRT1, SIRT2, SIRT3, SIRT4, SIRT5, SIRT6, or SIRT7. In an aspect, a disclosed composition can inhibit the expression of one or more of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of a combination of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of each of HDAC1, HDAC2, HDAC3, HDAC4, HDAC5, HDAC6, HDAC7, HDAC8, HDAC9, HDAC10, and HDAC11. In an aspect, a disclosed composition can inhibit the expression of one or more of SIRT1, SIRT2, SIRT3, SIRT4, SIRT5, SIRT6, and SIRT7. In an aspect, a disclosed composition can inhibit the expression of a combination of SIRT1, SIRT2, SIRT3, SIRT4, SIRT5, SIRT6, and SIRT7. In an aspect, a disclosed composition can inhibit the expression of one or more HDACs and can inhibit the expression of one or more SIRTs.

In an aspect of a disclosed method of decreasing c-myc expression, a disclosed composition comprising a compound of the formula:

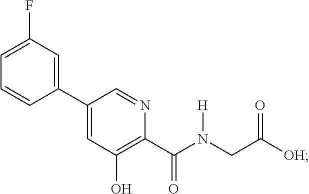 [SMILES: O=C(O)CNC(=O)c1ncc(-c2cccc(F)c2)cc1O]

or a pharmaceutically acceptable salt thereof can inhibit the expression of one or more prolyl hydroxylases. Prolyl hydroxylases, including non-mammalian homologs, are known to the art. In an aspect, a prolyl hydroxylase can comprise a human prolyl hydroxylase. In an aspect, a prolyl hydroxylases can comprise any known prolyl hydroxylase, such as, for example, PHD1, PHD2, and PHD3. In an aspect, a disclosed composition can inhibit the expression and/or activity of one or more prolyl hydroxylases. In an aspect, a disclosed composition can inhibit the expression and/or activity of PHD1. In an aspect, a disclosed composition can inhibit the expression and/or activity of PHD2. In an aspect, a disclosed composition can inhibit the expression and/or activity of PHD3. In an aspect, a disclosed composition can inhibit the expression and/or activity of a combination of PHD1, PHD2, and PHD3. In an aspect, a disclosed composition can inhibit the expression and/or activity of each of PHD1, PHD2, and PHD3.

An administering step of a disclosed method of decreasing c-myc expression can comprise any route of administration known to the art. For example, in an aspect, an administering step can comprise intraperitoneal administration. For example, in an aspect, an administering step can comprise oral administration. For example, in an aspect, an administering step can comprise intravenous administration.

In an aspect, a disclosed method of decreasing c-myc expression can comprise ameliorating one or more symptoms associated with aberrant DNA methylation, such as, for example, hypermethylation, of one or more gene promoters.

### iv) Method of Increasing Desmoplakin Expression

Disclosed herein is a method of increasing desmoplakin expression in a subject, comprising administering to a subject an effective amount of a composition comprising a compound of the formula:

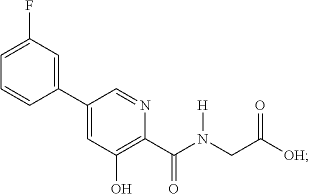 [SMILES: O=C(O)CNC(=O)c1ncc(-c2cccc(F)c2)cc1O]

or a pharmaceutically acceptable salt thereof. In an aspect, a disclosed method of increasing desmoplakin expression can comprise altering the methylation status of the desmoplakin promoter. In an aspect, a disclosed method of increasing desmoplakin expression can comprise determining the methylation status of the desmoplakin promoter. Methods of measuring or ascertaining the methylation status of a promoter such as the desmoplakin promoter are known to the art and are discussed above. In an aspect, determining the methylation status of the desmoplakin promoter can comprise comparing the methylation status of the desmoplakin promoter in an affected tissue of the subject to the methylation status of the desmoplakin promoter in an unaffected tissue of the subject. In an aspect, affected tissue can be a tumor or a cancer and unaffected tissue can be something other than a tumor or a cancer (i.e., a non-cancerous tissue or cell or sample). In an aspect, determining the methylation status of the desmoplakin promoter can comprise measuring the percent methylation of the desmoplakin promoter.

In an aspect of a method of increasing desmoplakin expression in a subject, a subject can be an unhealthy subject. In an aspect, an unhealthy subject can be a subject afflicted with or suffering from a disease, a condition, a disorder, or an illness.

In a disclosed method of increasing desmoplakin expression, a subject can have a disease or disorder that is not vascular leak. In a disclosed method of increasing desmoplakin expression, a subject can have a disease or disorder that is not retinopathy. In a disclosed method of increasing desmoplakin expression, a subject can have a disease or disorder that is not critical limb ischemia (CLI).

In an aspect of a disclosed method of increasing desmoplakin expression in a subject, the method can comprise identifying a subject in need thereof prior to the administering step.

In an aspect of a disclosed method of increasing desmoplakin expression, prior to the administering step, the desmoplakin promoter is hypermethylated. In other words, the c-myc promoter has a higher level of percent methylation. The level of percent methylation can be compared between two subjects, such as, for example, a subject diagnosed with or suspected of having a specific disease or disorder and a subject not diagnosed with or not suspected of having a specific disease or disorder. The level of percent methylation can be compared between within a subject, such as, for example, between an affected tissue or organ or cell and an unaffected tissue or organ or cell. In an aspect, an affected tissue or organ or cell can be a cancerous or tumorous tissue or organ or cell.

In an aspect of a disclosed method of increasing desmoplakin expression, after the administering step, there can be a change in the methylation status of the desmoplakin promoter. For example, in an aspect, a change in methylation status can comprise a decrease in the percent methylation of the desmoplakin promoter. If, after the administering step, the desired methylation status of the desmoplakin promoter is not achieved, then the method can comprise repeating the administration of an effective amount the composition. In an aspect, the desired methylation status can be a decrease in the percent methylation of the desmoplakin promoter. In an aspect, the administering step can be repeated prior to and after the methylation status is determined. In an aspect, the administering step can be repeated after the methylation status is determined. In an aspect, the administering step can be repeated one or more times, such as, for example, two, three, four, five, ten, fifteen, twenty, thirty, forty, fifty, or more times. In an aspect, the administering step can occur hourly, every 3 hours, every 6 hours, every 12 hours, every 18 hours, daily, weekly, bi-weekly, monthly, bi-monthly, yearly, bi-annually, every 5 years, or every 10 years of a subject's life. In an aspect, a disclosed method can comprise determining the methylation status of the desmoplakin promoter after a singular administering step. In an aspect, a disclosed method of increasing desmoplakin expression can comprise determining the methylation status of the desmoplakin promoter after some administering steps. In an aspect, a disclosed method can comprise determining the methylation status of the desmoplakin promoter after every administering step.

In a disclosed method of increasing desmoplakin expression, a subject can have cancer. In an aspect, the cancer can be a cancer that is caused by the hypermethylation of one or more gene promoters. For example, the cancer can be any cancer identified in Table 1. In an aspect, a subject can have breast cancer, triple negative breast cancer, melanoma, cervical cancer, or lung cancer. In an aspect, a subject can have one or more of breast cancer, triple negative breast cancer, melanoma, cervical cancer, and lung cancer. In an aspect, a disclosed method of increasing desmoplakin expression can comprise administering to a subject one or more anti-cancer agents.

In a disclosed method of increasing desmoplakin expression, desmoplakin gene expression can be increased. In a disclosed method of increasing desmoplakin expression, desmoplakin protein expression can be increased. In a disclosed method of increasing desmoplakin expression, desmoplakin gene expression and desmoplakin protein expression can be increased. In a disclosed method of increasing desmoplakin expression, an increase in desmoplakin gene and/or protein expression, for example, can inhibit metastases in the subject.

In an aspect, a disclosed method of increasing desmoplakin expression can comprise ameliorating one or more symptoms associated with aberrant DNA methylation, such as, for example, hypermethylation, of one or more gene promoters.

### v) Method of Inhibiting Metastases

Disclosed herein is a method of inhibiting metastases in a subject, comprising administering to a subject an effective amount of a composition comprising a compound of the formula:

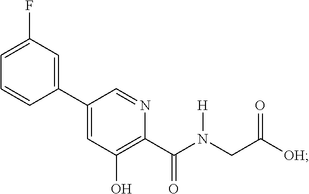 [SMILES: O=C(O)CNC(=O)c1ncc(-c2cccc(F)c2)cc1O]

or a pharmaceutically acceptable salt thereof; and modulating the DNA methylation status of one or more genes promoters. In an aspect of inhibiting metastases, modulating the DNA methylation status of one or more gene can comprise changing the methylation status of one or more gene promoters. In an aspect of inhibiting metastases, a disclosed method can comprise determining the methylation status of the one or more gene promoters.

In an aspect of a method of inhibiting metastases in a subject, a subject can be an unhealthy subject. In an aspect, an unhealthy subject can be a subject afflicted with or suffering from a disease, a condition, a disorder, or an illness.

In a disclosed method of inhibiting metastases, a subject can have a disease or disorder that is not vascular leak. In a disclosed method of inhibiting metastases, a subject can have a disease or disorder that is not retinopathy. In a disclosed method of inhibiting metastases, a subject can have a disease or disorder that is not critical limb ischemia (CLI).

Methods of measuring or ascertaining the methylation status of one or more gene promoters are known to the art. In an aspect, determining the methylation status of the one or more gene promoters can comprise comparing the methylation status of the one or more gene promoters in an affected tissue of the subject to the methylation status of the one or more gene promoters in an unaffected tissue of the subject. In an aspect, affected tissue can be a tumor or a cancer and unaffected tissue can be something other than a tumor or a cancer (i.e., a non-cancerous tissue or cell or sample). In an aspect, determining the methylation status of the one or more gene promoters can comprise measuring the percent methylation of the one or more promoters.

In an aspect of a disclosed method of inhibiting metastases in a subject, prior to the administering step, the one or more gene promoters are hypermethylated. In other words, the one or more gene promoters of interest have a higher level of percent methylation. The level of percent methylation can be compared between two subjects, such as, for example, a subject diagnosed with or suspected of having a specific disease or disorder and a subject not diagnosed with or not suspected of having a specific disease or disorder. The level of percent methylation can be compared between within a subject, such as, for example, between an affected tissue or organ or cell and an unaffected tissue or organ or cell. In an aspect, an affected tissue or organ or cell can be a cancerous or tumorous tissue or organ or cell.

In an aspect of a disclosed method of inhibiting metastases in a subject, the method can comprise identifying a subject in need thereof prior to the administering step.

In an aspect of a disclosed method of inhibiting metastases in a subject, after the administering step, there can be a change in the methylation status of the one or more gene promoters. For example, in an aspect, a change in methylation status can comprise a decrease in the percent methylation of the one or more gene promoters. If, after the administering step, the desired methylation status is not achieved, then the method can comprise repeating the administration of an effective amount the composition. In an aspect, the desired methylation status can be a decrease in the percent methylation of the one or more gene promoters. In an aspect, the administering step can be repeated prior to and after the methylation status is determined. In an aspect, the administering step can be repeated after the methylation status is determined. In an aspect, the administering step can be repeated one or more times, such as, for example, two, three, four, five, ten, fifteen, twenty, thirty, forty, fifty, or more times. In an aspect, the administering step can occur hourly, every 3 hours, every 6 hours, every 12 hours, every 18 hours, daily, weekly, bi-weekly, monthly, bi-monthly, yearly, bi-annually, every 5 years, or every 10 years of a subject's life. In an aspect, a disclosed method can comprise determining the methylation status of the one or more gene promoters after a singular administering step. In an aspect, a disclosed method of modulating DNA methylation can comprise determining the methylation status of the one or more gene promoters after some administering steps. In an aspect, a disclosed method can comprise determining the methylation status of the one or more gene promoters after every administering step.

In a disclosed method of inhibiting metastases in a subject, a gene promoter can comprise a desmoplakin (DSP) gene promoter, a c-myc gene promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a cytochrome P450, family 1, subfamily B, polypeptide 1 (CYP1B1) promoter, a deleted in liver cancer 1 (DLC1) promoter, an E-cadherin (CDH1) promoter, a fragile histidine triad (FHIT) promoter, a H-cadherin (CDH13) promoter, an O-6-methylguanine-DNA methyltransferase (MGMT) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, an Adenomatous polyposis coli (APC) promoter, an Amyloid beta A4 precursor protein-binding family A member 1 (APBA1) promoter, a cell adhesion molecule 1 (CADM1) promoter, a chemokine (C—X—C motif) ligand 12 (CXCL12) promoter, a methylenetetrahydrofolate reductase (NAD(P)H) (MTHFR) promoter, a mutL homolog 1 promoter, nonpolyposis type 2 (MLH1) promoter, a Ras association (RalGDS/AF-6) domain family member 2 (RASSF2) promoter, a secreted frizzled-related protein 1 (SFRP1) promoter, a transcription factor 21 (TCF21) promoter, or a vascular endothelial growth factor-A (VEGF-A) promoter.

A disclosed method of inhibiting metastases in a subject can comprise one or more of the following gene promoters: DSP, C-MYC, CDKN1C, CDKN2A, CDKN2B, CYP1B1, DLC1, CDH1, FHIT, CDH13, MGMT, OPCML, PAX5, PRDM2, RASSF1, APC, APBA1, CADM1, CXCL12, MTHFR, MLH1, RASSF2, SFRP1, TCF21, and VEGF-A.

In an aspect, a disclosed method of inhibiting metastases can comprise one or more gene promoters of one or more tumor suppressor genes (TSGs) as known to the art.

In a disclosed method of inhibiting metastases in a subject, a gene promoter can comprise an estrogen receptor gene (ER) promoter, a breast cancer 1 gene (BRCA1) promoter, an epithelial cadherin gene (E-cad) promoter, a TMS1 gene promoter, an insulin-like growth factor binding protein 7 gene (IGFBP7) promoter, a p16 promoter, a rentioic acid receptor gene (RARβ2) promoter, or a Ras association (RalGDS/AF-6) domain family member 1 gene (RASSF1A) promoter.

A disclosed method of inhibiting metastases in a subject can comprise one or more of the following gene promoters: ER, BRCA1, E-cad, TMS1, IGFBP7, RARβ2, and RASSF1A.

A disclosed method of modulating DNA methylation can comprise one or more of the following gene promoters: APBA1, APC, BRCA1, CADM1, CDH1, CDH13, CDKN1C, CDKN2A, CDKN2B, C-MYC, CXCL12, CYP1B1, DLC1, DSP, E-CAD, ER, FHIT, IGFBP7, MGMT, MLH1, MTHFR, OPCML, PAX5, PRDM2, RARβ2, RASSF1, RASSF1A, RASSF2, SFRP1, TCF21, TMS1, and VEGF-A.

In a disclosed method of inhibiting metastases, a subject can have cancer. In an aspect, the cancer can be a cancer that is caused by the hypermethylation of one or more gene promoters. For example, the cancer can be any cancer identified in Table 1. In an aspect, a disclosed method can comprise administering to a subject one or more anti-cancer agents. In an aspect, a subject can have breast cancer and the one or more gene promoters can comprise a desmoplakin (DSP) promoter. In an aspect, breast cancer can be triple negative breast cancer. In an aspect, a subject can have melanoma and the one or more gene promoters can comprise a desmoplakin (DSP) promoter. In an aspect of a disclosed method, a subject can have cervical cancer and the one or more gene promoters can comprise a desmoplakin (DSP) promoter or a c-Myc promoter. In an aspect, a subject can have lung cancer and the one or more gene promoters can comprise a desmoplakin (DSP) promoter, an Adenomatous polyposis coli (APC) promoter, an Amyloid beta A4 precursor protein-binding family A member 1 (APBA1) promoter, a cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

In an aspect, a disclosed method of inhibiting metastases can comprise ameliorating one or more symptoms associated with aberrant DNA methylation, such as, for example, hypermethylation, of one or more gene promoters.

### vi) Method of Synthesizing a Disclosed Compound

The disclosed compound, 6, which can also be referred to as AKB-6899, and ester prodrugs, for example, compound 5, can be prepared by the process outlined in Scheme I and further described in Example 1 herein below.

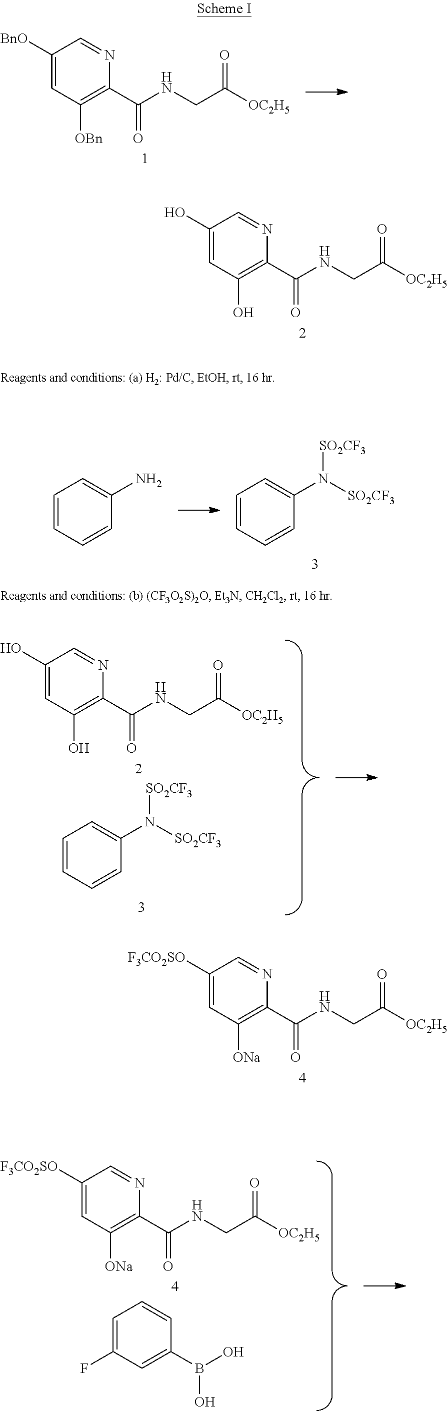

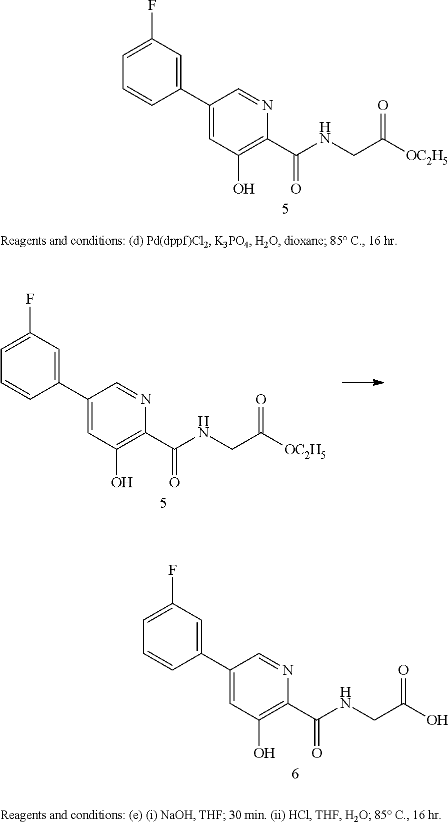 [SMILES: CCOC(=O)CNC(=O)c1ncc(-c2cccc(F)c2)cc1O ; CCOC(=O)CNC(=O)c1ncc(-c2cccc(F)c2)cc1O ; O=C(O)CNC(=O)c1ncc(-c2cccc(F)c2)cc1O]

### a. EXAMPLE 1

### {[5-(3-Fluorophenyl)-3-hydroxypyridine-2-carbonyl]amino}acetic acid (6), which can also be referred to as AKB-699

In the reactions described herein below, unless otherwise stated, temperatures are given in degrees Celsius (°C.); operations were carried out at room or ambient temperature, “room temperature,” “rt,” or “RT” (typically a range of from about 18° C. to about 25° C.; evaporation of solvent was carried out using a rotary evaporator under reduced pressure (typically, 4.5-30 mm Hg) with a bath temperature of up to 60° C.; the course of reactions was typically followed by thin layer chromatography (TLC); products exhibited satisfactory ¹H NMR, HPLC, and/or LC-MS (GC-MS) data; and the following conventional abbreviations are also used: L (liter(s)), mL (milliliters), mmol (millimoles), g (grams), and mg (milligrams). Unless specified otherwise, all solvents and reagents were purchased from suppliers and used without further purification. Reactions were conducted under a blanket of nitrogen unless otherwise stated. Compounds were visualized under UV lamp (254 nm). ¹H NMR spectra were recorded on a 300 MHz NMR.

Preparation of [(3,5-dihydroxypyridine-2-carbonyl)-amino]-acetic acid ethyl ester (2): To a 20 L round-bottomed flask was charged nitrogen and palladium on carbon (10% Pd/C) (100 g, 60% wet paste) and ethanol (12 L), followed by the addition of [(3,5-bis-benzyloxypyridine-2-carbonyl)-amino]-acetic acid ethyl ester, 1, (1000 g, 2.378 mol). The resulting mixture was subjected to a vacuum-nitrogen purge cycle three times and a vacuum-hydrogen purge cycle three times. Hydrogen atmosphere was introduced and the reaction mixture was stirred at 1-25° C. until the completion of the reaction by TLC analysis. The reaction typically lasted 2-3 hours and a vigorous stirring was important to complete the reaction. The reaction system was then subjected to a vacuum-nitrogen purge cycle to remove hydrogen from the system. The reaction mixture was filtered and the filter-cake was washed with ethanol (2 L). The combined filtrate was concentrated on a rotary evaporator at up to 45° C. bath temperature to a constant weight to provide 558 g (97.7% yield) of the desired product as an off-white solid. MP: 138-140° C.; MS(ESI+): m/z 241 (M+1); ¹H NMR (300 MHz, DMSO-d₆) 12.28 (s, 1H), 10.79 (s, 1H), 9.09-9.05 (t, J=6 Hz, 1H), 7.76-7.71 (d, J=2.4 Hz, 1H), 6.68-6.67 (d, J=2.1 Hz, 1H), 4.15-4.08 (q, J=6.9 Hz, 2H), 4.02-4.00 (d, J=6.3 Hz, 2H), 1.22-1.17 (t, J=6.9 Hz, 2H).

Preparation of Ar-phenylbis(trifluormethane-sulfinimide) (3): To a 10 L found-bottomed flask was charged aniline (232.5 g, 2.5 mol), triethylamine (505 g, 5 mol) and dichloromethane (5 L). The resulting mixture was cooled with an ice bath. Trifluoromethanesulfonic anhydride (1410 g, 5 mol) in dichlolormethane (1 L) was added dropwise. The reaction mixture was allowed to warm to RT and stirred overnight. The reaction was then added to crushed ice (4 kg) while stirring. The resulting biphasic mixture was separated. The organic layer was washed with brine (2 L×2), dried over Na₂SO₄, filtered and concentrated to form a crude solid product. The crude solid was washed with ethanol to produce 767 g (86% yield) of the desired product as a white solid. MP: 96-98° C.; ¹H NMR (300 MHz, CDCl₃) δ 7.64-7.51 (m, 3H), 7.44-7.42 (m, 2H).

Preparation of [(3-hydroxy-5-trifluoromethanesulfonyloxypyridine-2-carbonyl)-amino]-acetic acid ethyl ester sodium salt (4): To a 20 L round-bottomed flask was charged [(3,5-dihydroxy-pyridine-2-carbonyl)-amino]-acetic acid ethyl ester, 2, (860 g, 3.58 mol) and ethanol (11 L). The mixture was stirred to form a solution at 10 to 20° C. Triethylamine (602 mL, 4.3 mol) was added. The resulting mixture was cooled to 0-5° C. and N-phenylbis(trifluormethane-sulfinimide), 3, (1406 g, 3.94 mol) was added. After addition, the reaction mixture was warmed to 35 to 40° C. and stirred overnight. TLC analysis indicated that the reaction was complete. The reaction mixture was then concentrated by rotary evaporation at up to 40° C. bath temperature. The residue (oily solid) was treated with toluene (4.5 L) and concentrated to approximately 4.5 L. The toluene solvent swap was repeated until residue ethanol level became less than 0.5% by ¹H NMR analysis. The toluene solution was treated with 10% w/w aqueous Na₂CO₃ solution (5.5 L, 1.3 eq.). The resulting slurry was filtered and the filter cake was washed with water (2×2 L) and then a mixture of toluene/TBME (1:2) (2×2 L). The solid product was dried to afford 1156 g (82% yield) of the desired product as a white solid. MS(ESI+): m/z 373 (M+1); ¹H NMR (300 MHz, DMSO-d₆) 12.13 (1 H, s), 7.43-7.42 (d, J=2.1 Hz, 1H), 6.72-6.71 (d,J=2.1 Hz, 2H), 4.12-4.05 (m, 4H), 1.21-1.15 (t, J=6.9 Hz, 3).

Preparation of {[5-(3-fluorophenyl)-3-hydroxypyridine-2-carbonyl]amino}acetic acid ethyl ester (5): To a 5 L round-bottomed flask was charged [(3-hydroxy-5-trifluoromethanesulfonyloxypyridine-2-carbonyl)-amino]-acetic acid ethyl ester sodium salt, 4, (310 g, 0.78 mol), 1,4-dioxane (3 L) and water (150 mL). The solution was subjected to a vaccum-nitrogen purge cycle, followed by the addition of potassium phosphate (50 g, 0.234 mol) and 3-fluorophenylboronic acid (163 g, 1.17 mol). After addition, the vacuum-nitrogen purge cycle was repeated once. 1,1-Bis(diphenyl-phosphino)ferrocenepalladium (II) chloride CH₂Cl₂ complex (72 g, 0.088 mol, 0.11 eq.) was then added. After another vacuum-nitrogen purge cycle, the reaction mixture was then heated to 75 to 85° C. The progress of the reaction was monitored by TLC. The reaction was complete after 14-16 hours. The reaction was cooled to 15 to 25° C. and concentrated by rotary evaporation at up to 45° C. bath temperature until solvent collection had ceased. The residue was treated with an aquous solution of HCl (1M, 1.5 L) and ethyl acetate (1.5 L) and stirred for 30 minutes at room temperature. The layers were then separated. The organic layer was washed with water (1.5 L), brine (1.5 L), dried over Na₂SO₄, filtered and concentrated. The crude product was purified by silica gel column chromatography (hexane/ethylacetate/acetic acid: 3:1:0.01 by vol/vol) to afford 226 g (90% yield) of the desired product. MS(ESI+): m/z 319 (M+1); ¹H NMR (300 MHz, CDCl3) 11.88 (s, 1H), 8.44 (s, 1H), 8.32-0.31 (d, J=1.5 Hz, 1H), 7.51-7.44 (m, 2H), 7.40-7.37 (m, 1H), 7.32-7.27 (m, 1H), 7.17-7.13 (t, J=6.6 Hz, 1H), 4.33-4.25 (m, 4H), 1.36-1.31 (t, J=7.2 Hz, 3H).

Preparation of {[5-(3-fluorophenyl)-3-hydroxypyridine-2-carbonyl]amino}acetic acid (6): To a slurry of {[5-(3-fluorophenyl)-3-hydroxypyridine-2-carbonyl]amino}acetic acid ethyl ester, 5, (226 g, 0.71 mol) in THF (1 L) at room temperature was added an aqueous solution of sodium hydroxide (1 M, 2 L) while maintaining the internal reaction temperature below 25° C. The progress of the reaction was monitored by TLC. After 20-30 minutes, the reaction was completed. The pH of the reaction solution was adjusted using concentrated HCl to 5-5.5 while maintaining the internal temperature below 25° C. The reaction mixture was filtered to remove insoluble matter and the filtrate was concentrated by rotary evaporation at up to 40° C. bath temperature until all THF was removed. The resulting solid was collected by vacuum filtration and washed with water (1 L). The solid was then dissolved in a mixture of water (1.5 L) and THF (1.5 L) at room temperature. The pH was adjusted from approximately 5 to approximately 2-2.25 with concentrated HCl. The resulting mixture was stirred for 30 minutes, after which time the pH was confirmed in the range of 2-2.5. The biphasic mixture was concentrated by rotary evaportation at up to 40° C. bath temperature until the removal of THF ceased. The resulting solid was filtered, washed with water (2×1 L), and dried to afford 115 g (55.8% yield) of the desired product as a white solid. MP: 182-184° C.; MS(ESI−): m/z 289 (M−1); ¹H NMR (300 MHz, DMSO-d₆) δ 12.90 (s, 1H), 12.38 (s, 1H), 9.39-9.37 (t, J=6.3 Hz, 1H), 8.55 (s, 1H), 7.80-7.67 (m, 2H), 7.59-7.52 (m, 1H), 7.34-7.27 (m, 1H), 4.02-3.99 (m, 2H), 3.51 (s, 1H).

The amide prodrugs of the disclosed HIF-2α stabilizer can be prepared by the process outlined in Scheme II and further described in Example 2 herein below.

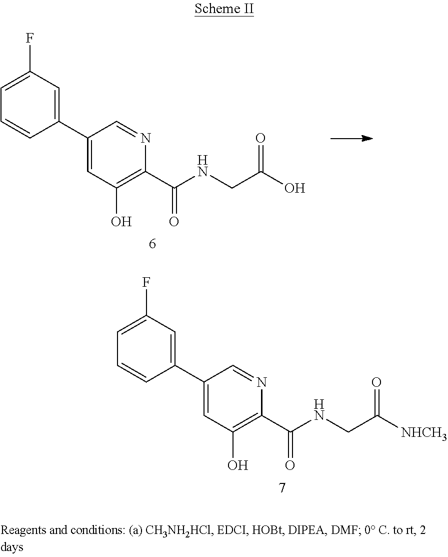 [SMILES: O=C(O)CNC(=O)c1ncc(-c2cccc(F)c2)cc1O ; CNC(=O)CNC(=O)c1ncc(-c2cccc(F)c2)cc1O]

### b. EXAMPLE 2

### 5-(3-Fluorophenyl)-N-(2-methylamino-2-oxoethyl)-3-hydroxypyridin-2-yl amide (7)

Preparation of 5-(3-fluorophenyl)-N-(2-methylamino-2-oxoethyl)-3-hydroxypyridin-2-yl amide (7): To a solution of {[5-(3-fluorophenyl)-3-hydroxypyridine-2-carbonyl]-amino}acetic acid, 6, (2.9 g, 10 mmol) in DMF (50 mL) at room temperature under N₂ is added 1-(3-dimethylamino-propyl)-3-ethylcarbodiimide (EDCI) (2.33 g, 14.4 mmol), 1-hydroxybenzotriazole (HOBt) (1.35 g, 10 mmol) and diisopropylethylamine (DIPEA) (15.65 mL, 30 mmol). The reaction is stirred for 5 minutes then methylamine hydrochloride (0.9 g, 130 mmol) is added. After stirring for 2 days, the solvent is removed under reduced pressure and the residue partitioned between CH₂Cl₂ and H₂O. The organic layer is separated, washed with sat. NaCl, dried (Na₂SO₄), filtered and concentrated under reduced pressure. The crude product is purified over silica (MeOH:CH₂Cl₂ 1:99) to afford the desired compound.

The following describes a further process for preparing the disclosed HIF-2α stabilizer and prodrugs thereof. In Scheme III the process for preparing an example of an ester prodrug is outlined and described in Example 3.

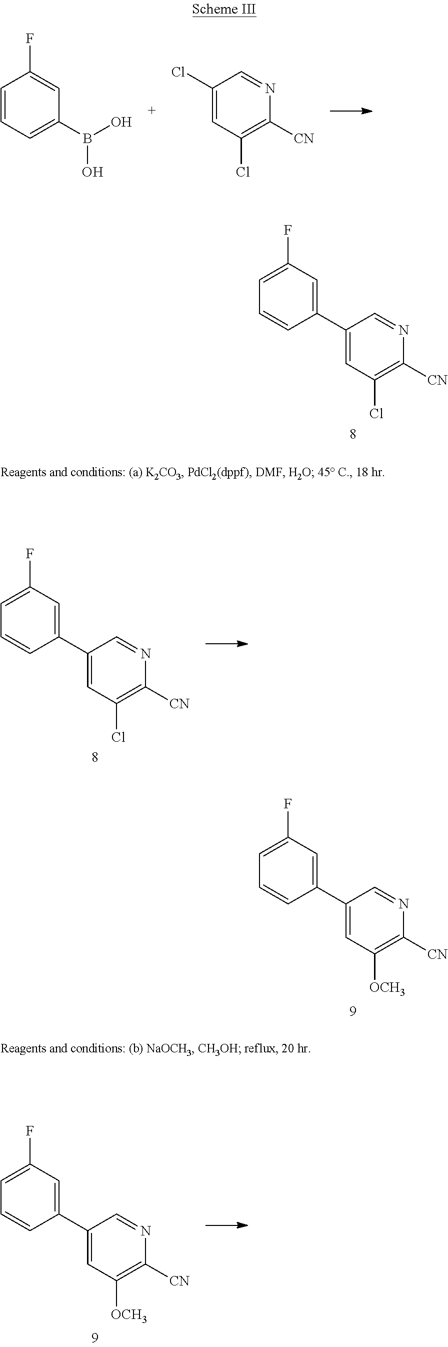 [SMILES: OB(O)c1cccc(F)c1 ; N#Cc1ncc(Cl)cc1Cl ; N#Cc1ncc(-c2cccc(F)c2)cc1Cl ; N#Cc1ncc(-c2cccc(F)c2)cc1Cl ; COc1cc(-c2cccc(F)c2)cnc1C#N ; COc1cc(-c2cccc(F)c2)cnc1C#N]

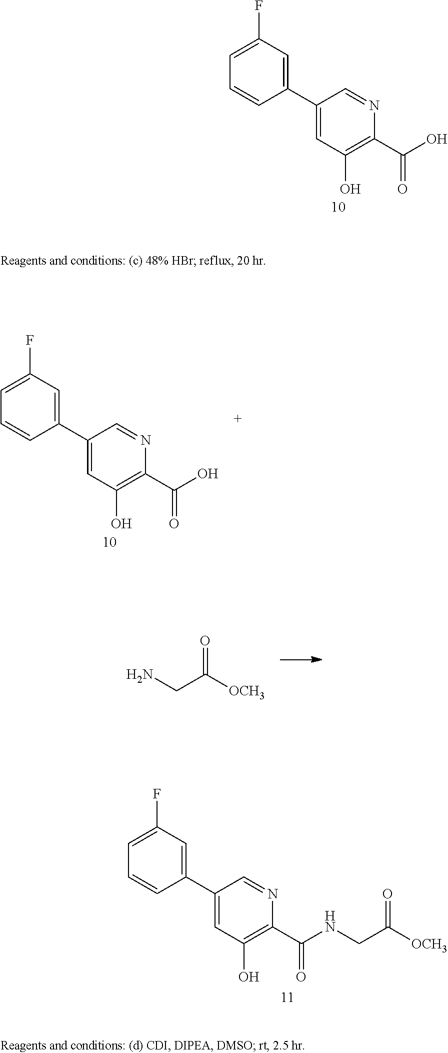 [SMILES: O=C(O)c1ncc(-c2cccc(F)c2)cc1O ; O=C(O)c1ncc(-c2cccc(F)c2)cc1O ; COC(=O)CN ; COC(=O)CNC(=O)c1ncc(-c2cccc(F)c2)cc1O]

### c. EXAMPLE 3

### Methyl {[5-(3-fluorophenyl)-3-hydroxypyridin-2-carbonyl]amino}acetate (11)

Preparation of 5-(3-fluorophenyl)-3-chloro-2-cyanopyridine (8): To a 100 mL round bottom flask that is adapted for magnetic stirring and equipped with a nitrogen inlet is charged (3-fluorophenyl)boronic acid (4.48 g, 32 mmol), 3,5-dichloro-2-cyanopyridine (5.8 g, 34 mmol), K₂CO₃ (5.5 g, 40 mmol), [1,1′-bis(diphenyphosphino)ferrocene]dichloro-palladium(II) [PdCl₂(dppf)] (0.1 g, 0.13 mmol), dimethylformamide (50 mL) and water (5 mL). The reaction solution is agitated and heated to 45° C. and held at that temperature for 18 hours after which the completeness of the reaction can be determined by the absence of the starting material 3,5-dichloro-2-cyanopyridine via TLC using ethyl acetate/methanol (4:1) as the mobile phase and UV 435 nm to visualize any remaining starting material. The reaction solution is then cooled to room temperature and the contents partitioned between ethyl acetate (250 mL) and saturated aqueous NaCl (100 mL). The organic phase is isolated and washed a second time with saturated aqueous NaCl (100 mL). The organic phase is dried for 4 hours over MgSO₄, the MgSO₄ is removed by filtration and the solvent is removed under reduced pressure. The residue that remains is then slurried in methanol (50 mL) at room temperature for 20 hours. The resulting solid is collected by filtration and washed with cold methanol (50 mL) then hexanes (60 mL) and dried to afford desired product.

Preparation of 5-(3-fluorophenyl)-3-methoxy-2-cyanopyridine (9): To a 500 mL round bottom flask adapted for magnetic stirring and fitted with a reflux condenser and nitrogen inlet is charged 5-(3-fluorophenyl)-3-chloro-2-cyanopyridine, 8, (9.28 g, 40 mmol), sodium methoxide (13.8 mL, 60 mmol) and methanol (200 mL). With stirring, the reaction solution is heated to reflux for 20 hours. The reaction can be determined to be complete due to the disappearance of 5-(3-fluorophenyl)-3-chloro-2-cyanopyridine as measured by TLC analysis using hexane/ethyl acetate (6:3) as the mobile phase and UV 435 nm to visualize the reaction components. The reaction mixture is cooled to room temperature and combined with water (500 mL). The mixture is cooled to 0° C. to 5° C. and stirred for 3 hours. The resulting solid is collected by filtration and washed with water, then hexane. The resulting cake is then dried in vacuo at 40° C. to afford the desired product.

Preparation of 5-(3-fluorophenyl)-3-hydroxypyridine-2-carboxylic acid (10): To a 50 mL round bottom flask adapted for magnetic stirring and fitted with a reflux condenser is charged 5-(3-fluorophenyl)-3-methoxy-2-cyanopyridine, 9, (0.912 g, 4 mmol) and a 48% aqueous solution of HBr (10 mL). While being stirred, the reaction solution is heated to reflux for 20 hours. The reaction can be determined to be complete due to the disappearance of 5-(3-fluorophenyl)-3-methoxy-2-cyanopyridine as measured by TLC analysis using hexane/ethyl acetate (6:3) as the mobile phase and UV 435 nm to visualize the reaction components. The reaction is then cooled to 0° C. to 5° C. with stirring and the pH is adjusted to approximately 2 by the slow addition of 50% aqueous NaOH. Stirring is then continued at 0° C. to 5° C. for 3 hours. The resulting solid is collected by filtration and washed with water, then hexane. The resulting cake is dried in vacuo at 40° C. to afford the desired product.

Preparation of methyl {[5-(3-fluorophenyl)-3-hydroxypyridin-2-carbonyl]amino}-acetate (11): To a 50 mL round bottom flask adapted for magnetic stirring and fitted with a nitrogen inlet tube is charged 5-(3-fluorophenyl)-3-hydroxypyridine-2-carboxylic acid, 10, (0.932 gm, 4 mmol), N,N′-carbonyldiimidazole (CDI) (0.97 g, 6 mmol) and dimethyl sulfoxide (5 mL). The reaction mixture is stirred at 45° C. for about 1 hour then cooled to room temperature. Glycine methyl ester hydrochloride (1.15 g, 12 mmol) is added followed by the drop wise addition of diisopropylethylamine (3.2 mL, 19 mmol). The mixture is then stirred for 2.5 hours at room temperature after which water (70 mL) is added. The contents of the reaction flask are cooled to 0° C. to 5° C. and 1N HCl is added until the solution pH is approximately 2. The solution is extracted with dichloromethane (100 mL) and the organic layer dried over MgSCU for 16 hours. Silica gel (3 g) is added and the solution slurried for 2 hours after which the solids are removed by filtration. The filtrate is concentrated to dryness under reduced pressure and the resulting residue is slurried in methanol (10 mL) for two hours. The resulting solid is collected by filtration and washed with cold methanol (20 mL) then hexane and the resulting cake is dried to afford the desired product.

Ester prodrug methyl {[5-(3-fluorophenyl)-3-hydroxypyridin-2-yl]amino}acetate, 11, can be converted to the disclosed HIF-2α stabilizer, {[5-(3-Fluorophenyl)-3-hydroxypyridine-2-carbonyl]amino}acetic acid, 6, by the procedure outlined in Scheme I step (e) and described in Example 1.

Scheme IV herein below outlines and Example 4 describes a further non-limiting example of a procedure for an amide prodrug of the disclosed HIF-2α stabilizer.

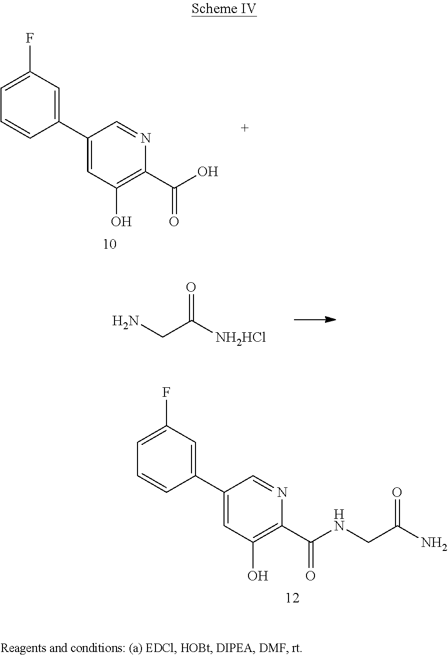

### d. EXAMPLE 4

### 5-(3-Fluorophenyl)-N-(2-amino-2-oxoethyl)-3-hydroxylpyridin-2-yl amide (12)

Preparation of 5-(3-fluorophenyl)-N-(2-amino-2-oxoethyl)-3-hydroxylpyridin-2-yl amide (6): To a solution of 5-(3-fluorophenyl)-3-hydroxypyridine-2-carboxylic acid, 10, (699 mg, 3 mmol) in DMF (20 mL) at room temperature under is added 1-(3-dimethyl-aminopropyl)-3-ethylcarbodiimide (EDCI) (0.925 g, 5.97 mmol) and 1-hydroxybenzo-triazole (HOBt) (0.806 g, 5.97 mmol). The resulting solution is stirred for 15 minutes then 2-aminoacetamide hydrochloride (0.66 g, 5.97 mmol) and diisopropylethylamine (1.56 ml, 8.96 mmol) are added. The reaction is monitored by TLC and when the reaction is complete the reaction mixture is concentrated under reduced pressure and H₂O added. The desired product can be isolated by normal work-up.

The present disclosure also includes pharmaceutically acceptable salts of the disclosed stabilizer. The following is a non-limiting example of the preparation of a pharmaceutically acceptable salt as depicted in Scheme V.

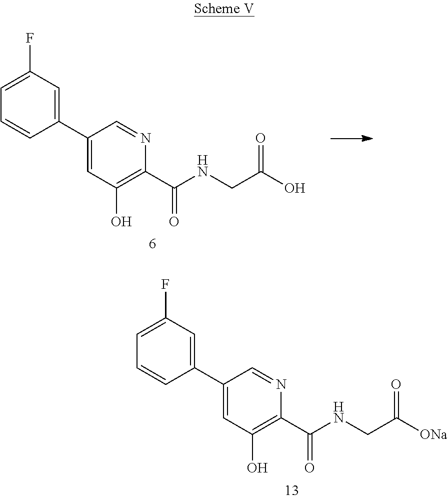

### e. EXAMPLE 5

### Sodium {[5-(3-fluorophenyl)-3-hydroxypyridine-2-carbonyl]amino}acetate (13)

To a vial containing NaHCO₃ (41.09 mg) is added a solution of {[5-(3-fluoro-phenyl)-3-hydroxypyridine-2-carbonyl]-amino}acetic acid (6) in acetone (0.64 mL of a 400 mg sample in 5.12 mL). The solution is stirred and the desired product isolated by concentration in vacuo.

### D. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention. Efforts have been made to ensure accuracy with respect to numbers (e.g., amounts, temperature, etc.), but some errors and deviations should be accounted for.

### i) Effect of AKB-6899 on Desmoplakin Expression and Desmosome Function

In these experiments, whether the inhibition of PHD3 caused by AKB-6899 treatment increases desmoplakin mRNA and protein expression in MDA-MB-231 human triple negative breast cancer cells (in vitro) is evaluated. Furthermore, whether the inhibition of PHD3 effected by AKB-6899 treatment affects desmosome function is evaluated.

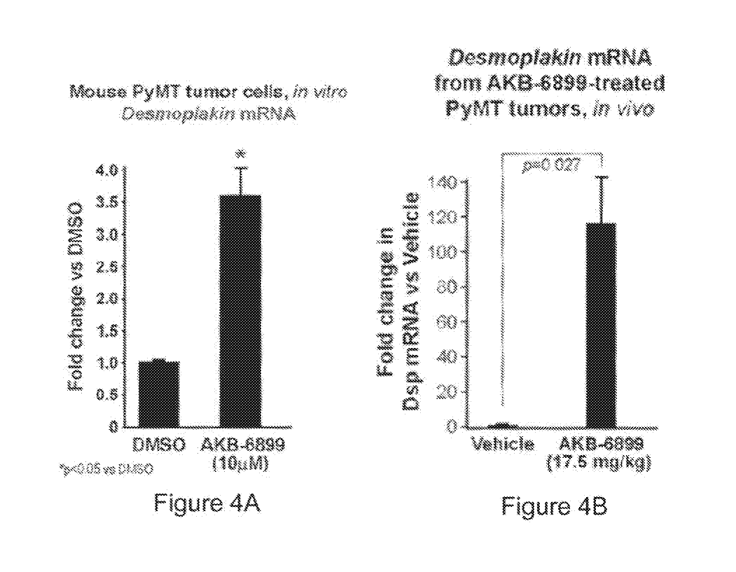

When compared to control animals (i.e., receiving DMSO), PyMT breast tumor-bearing mice treated with AKB-6899 (17.5 mg/kg) had a significant increase in DSP mRNA expression (i.e., ˜118 fold increase in AKB-6899 treated animals) (FIG. 4B). Desmoplakin (DSP) is the major protein component of the desmosome located in the inner membrane of epithelial cells where it binds desmosomal cadherins to maintain cell-to-cell adhesion. DSP is regulated by ER/PR expression (Maynadier et al. 2012; Ahmed et al., 1995; Pang et al., 2004). Clinically, expression of DSP is higher in normal mammary epithelium. Loss of DSP correlates with less differentiated breast tumors and increased lymph node involvement. Loss of DSP is significantly inversely correlated with ki-67 staining (Davies et al., 1999). Functional desmoplakin that is incorporated into the desmosome is not phosphorylated at serine 165/166. Phosphorylation at ser165/166 by protein kinase C (PKC) causes desmoplakin to disassemble from the desmosome. The loss of desmosome function and decreased adherence junctions are markers that can inform tumor staging, prognosis, and treatment planning as this transition eliminates therapeutic effectiveness and increases the likelihood of poor clinical outcome (Dusek et al., 2011). Further, loss of DSP is a marker in the Epithelial-to-Mesenchymal Transition (EMT) process as it binds to adherence proteins that also translocate to the nucleus and activate the Wnt/β-catenin pathway (Yang et al., 2012).

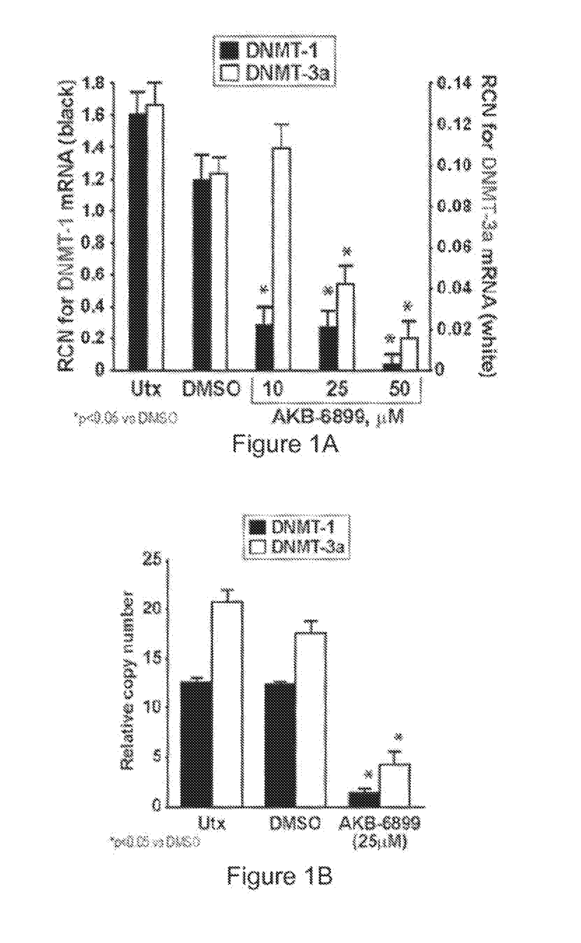

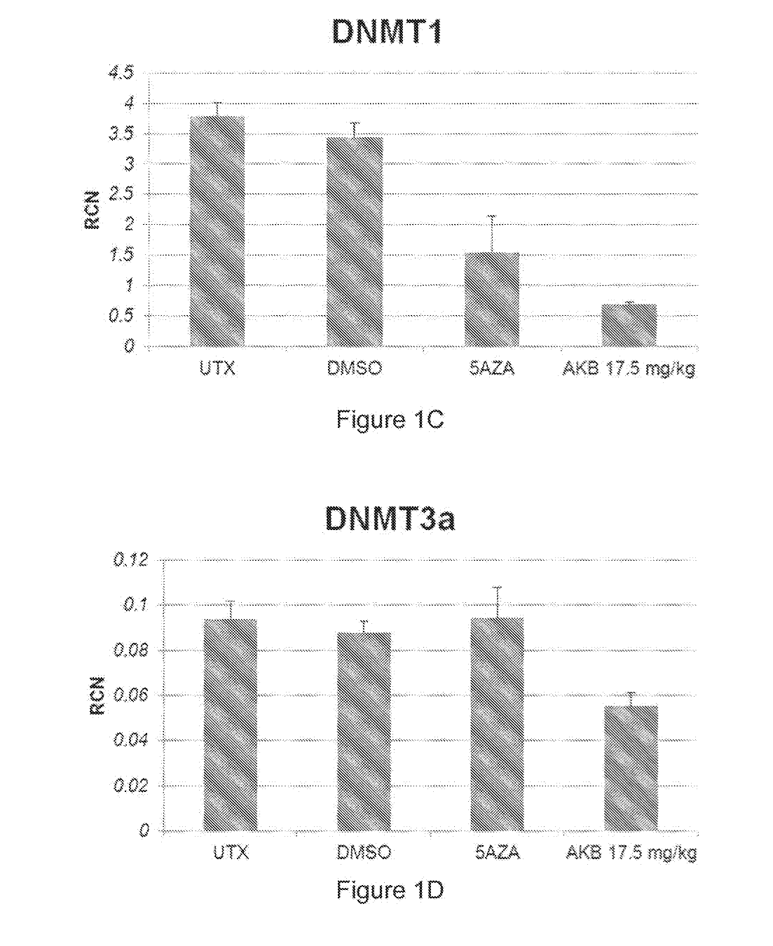

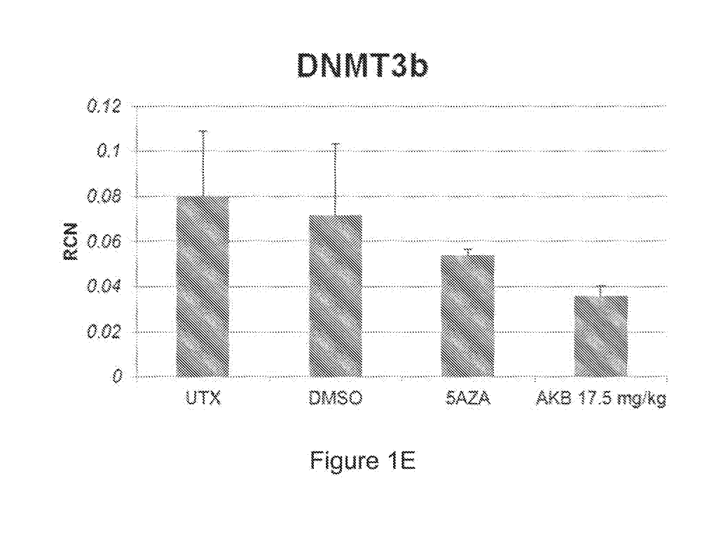

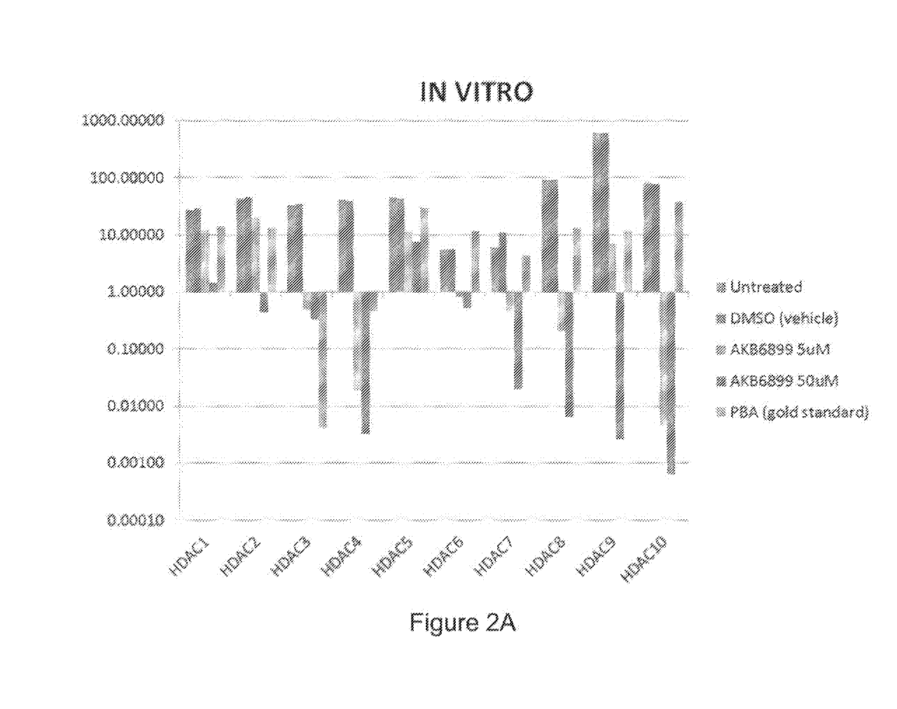

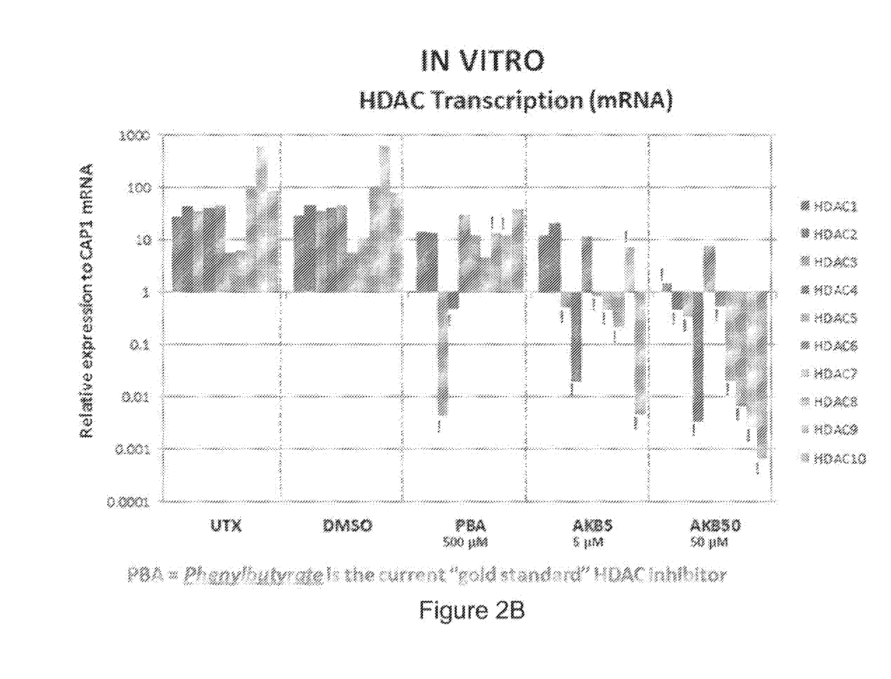

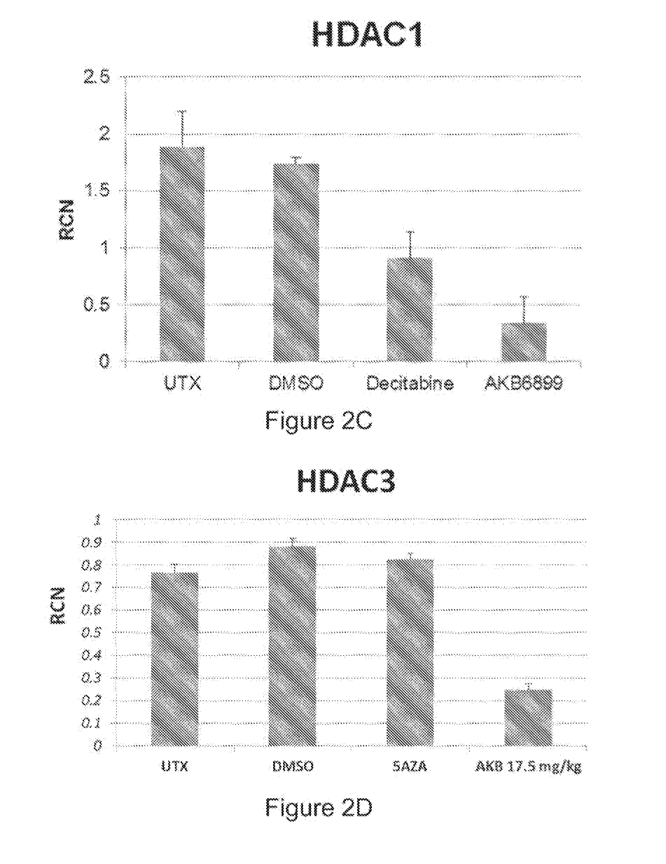

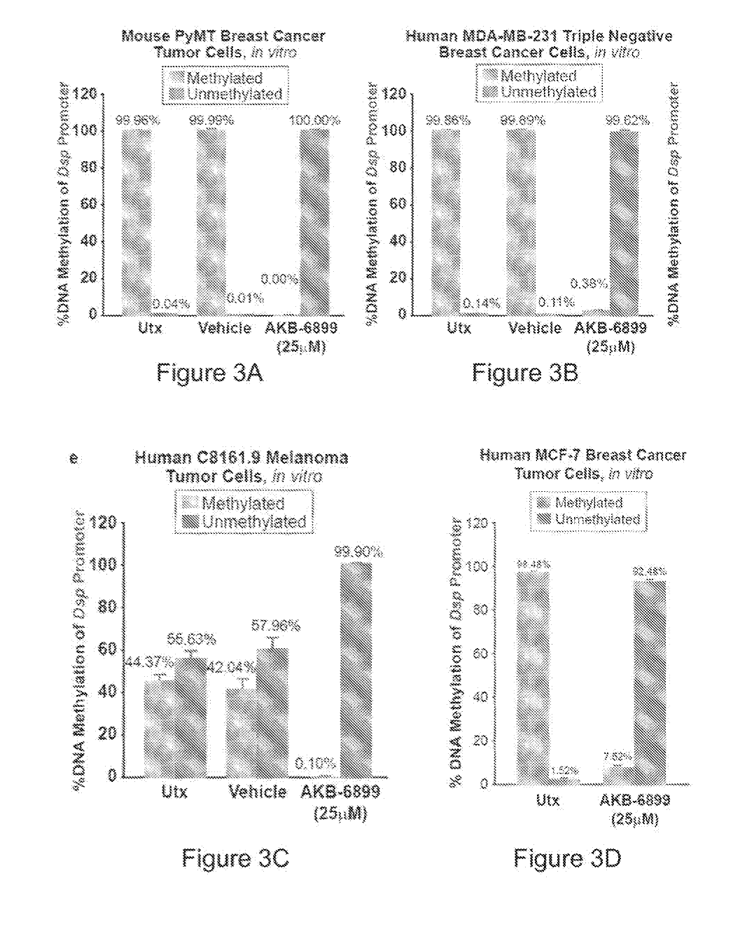

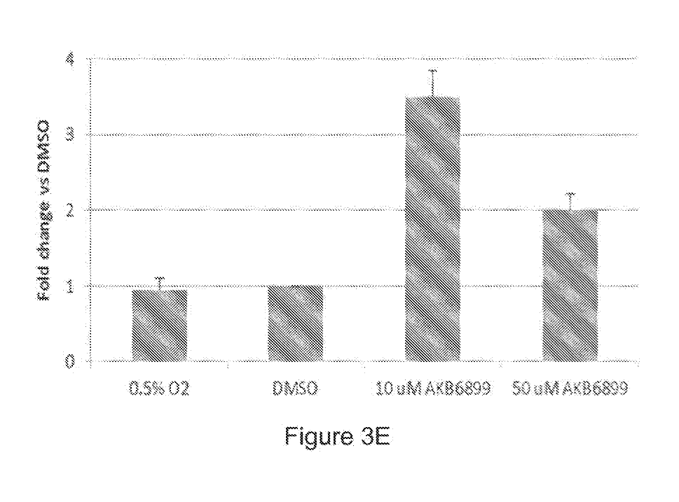

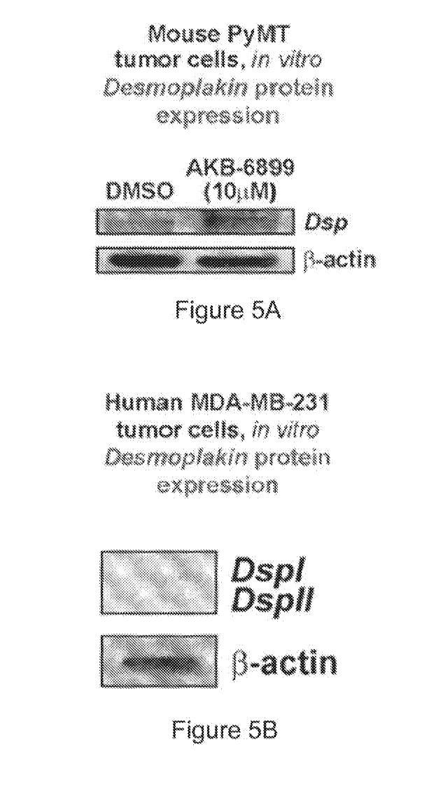

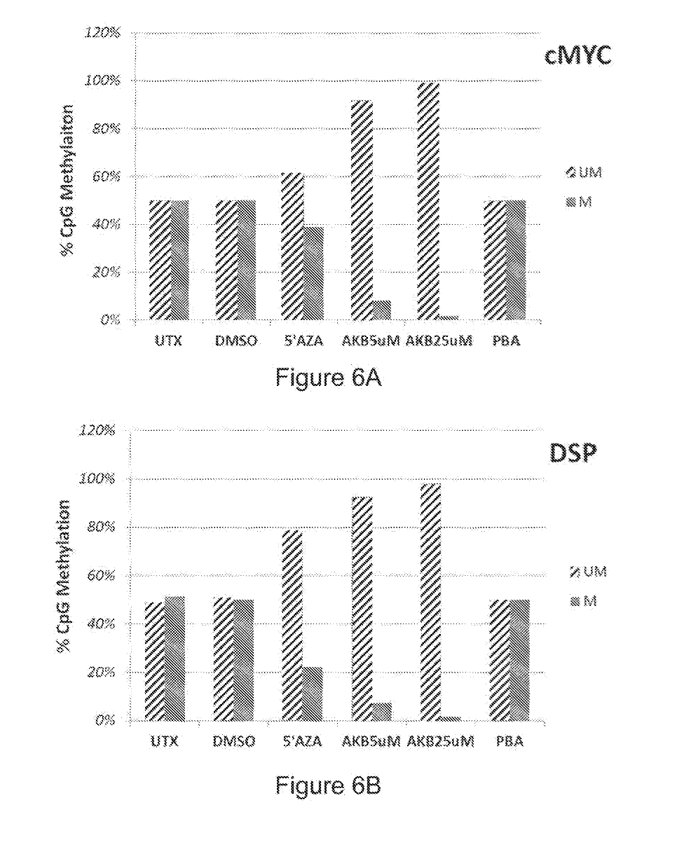

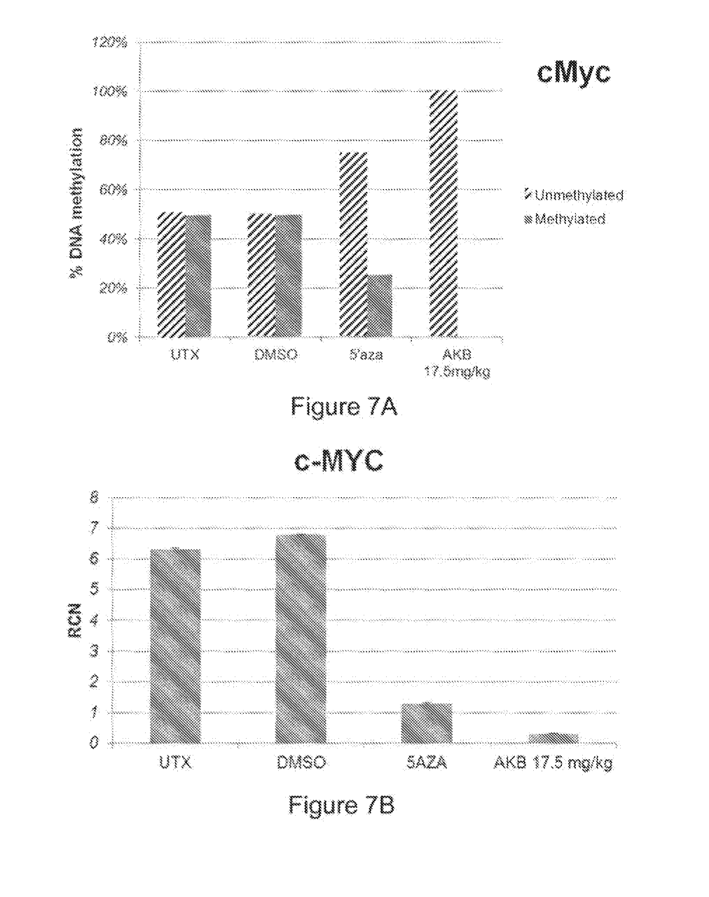

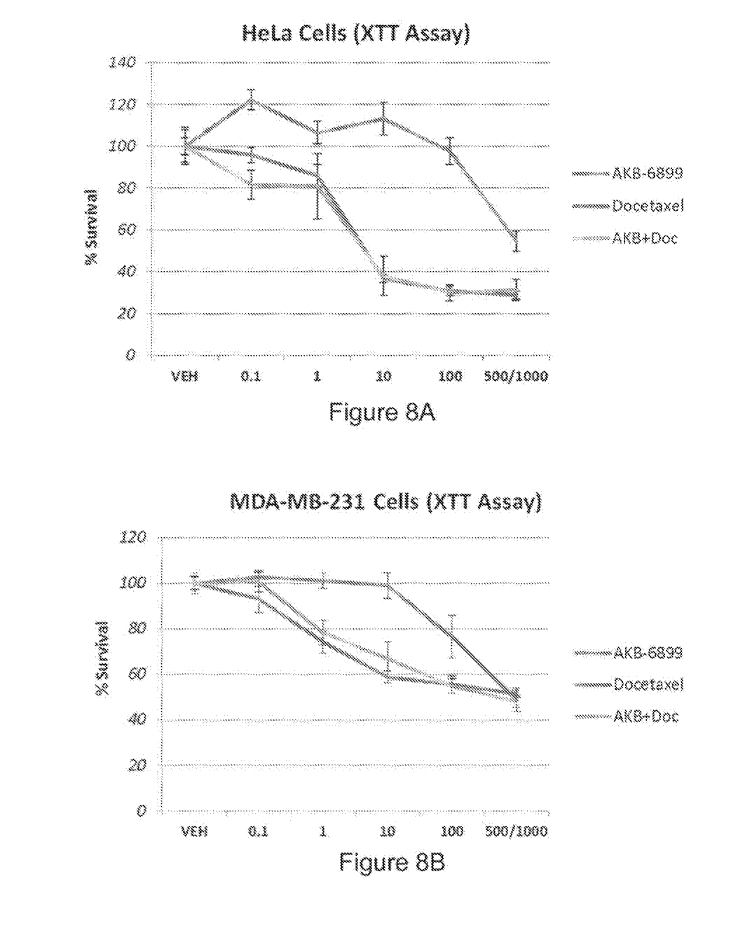

Because DSP is regulated by ER/PR expression, and because late-stage PyMT tumors lose ER/PR-positivity and heavily metastasize to the lymph nodes and lungs (Lin et al., 2003), the effects of AKB-6899 on more global events that regulate gene expression was evaluated. PyMT tumor cells in culture were treated with DMSO or AKB-6899. Methylation-specific PCR for CpG islands in the desmoplakin promoter were performed. The data show almost complete methylation of the DSP promoter in untreated cells (Utx or UTX) and DMSO-treated (vehicle) cells. Conversely, the cells treated with AKB-6899 were entirely unmethylated (FIG. 3A). Trypan Blue confirmed no cell death in AKB-6899 treated cells when compared to control cells. The experiment was repeated and RNA was isolated from the cells. RT-PCR was performed and the regulation of DNA methyltransferases (DNMTs) was evaluated. There was no difference in the mRNA level of DNMT-3b, but significant decreases in the mRNA level of DNMT-1 and DNMT-3a (i.e., the major DNMTs that regulate methylation) were recorded. (FIG. 1A). The decrease in mRNA levels of DNMT-1 and DNMT-3a increased the mRNA level of DSP (FIG. 4A) and increased desmoplakin protein expression (FIG. 5A).

To determine whether the decrease in DNMT expression and the increase in DSP expression were relevant in human cancer, the level of desmoplakin expression in MDA-MB-231 human triple negative breast cancer cells was determined. Western blot analysis revealed that MDA-MB-231 human triple negative breast cancer cells, which also lack ER/PR, did not express the protein (FIG. 5B). The MDA-MB-231 human triple negative breast cancer cells were evaluated for DNA methylation and it was determined that these cells are significantly hypermethylated at the DSP CpG islands. AKB-6899 induced almost entire demethylation of the DSP CpG islands. (FIG. 3B). Similar to the PyMT tumor cells, AKB-6899 significantly decreased the mRNA levels of DNMT-1 and DNMT-3a in the MDA-MB-231 human tumor cells (FIG. 1B).

Next, whether AKB-6899 increases the mRNA level of desmoplakin and protein in human breast cancer cells is evaluated. MDA-MB-231 human triple negative breast cancer (TNBC) cells are cultured and transfected with (i) human siRNA targeting prolyl hydroxylase-3 (PHD3) or (ii) a scrambled control siRNA for 24 hours (see Eubank et al., 2011 for siRNA strategies). The transfected cells are either left untreated (Utx) or are treated with AKB-6899 (1 μM, 10 μM, and 25 μM) or DMSO (vehicle). Treatment lasts for 24, 48, or 72 hrs. Toxicity effects (e.g., cell death) are evaluated using a Trypan Blue exclusion assay, and if necessary, treatment concentrations are adjusted. Using a first sample, the cells are pelleted for DNA isolation followed by methylation-specific PCR with primers targeting the desmoplakin promoter. For example, the experiments disclosed herein utilized the QIAGEN EpiTect Methyl Signature PCR Kits. Using a second sample, the cells are subjected to Trizol homogenization, total RNA purification, cDNA synthesis, and RT-PCR. The mRNA level of DSPI and DSPII mRNA is measured. The mRNA level of PHD3 following knockdown by siPHD3 is evaluated. Using a third sample, the cells are lysed with cell lysis buffer containing protease inhibitors. Lysates are immunostained with desmoplakin I/II antibodies and PHD3 antibodies for western blot analysis and the expression of desmoplakin is determined. The decrease in the expression of PHD3 protein is confirmed. In both treated and untreated cells, the absence or the presence of PHD3 mRNA is compared to (i) the percent DNA methylation of the desmoplakin CpG islands, (ii) desmoplakin mRNA expression, and (iii) desmoplakin protein expression.

Whether AKB-6899 enhances desmosome function in triple negative breast cancer is evaluated. MDA-MB-231 human triple negative breast cancer cells are cultured while some cells are not treated. Other cells are treated with DMSO (vehicle) or treated with AKB-6899 (1 μM, 10 μM, and 25 μM) (see Roda et al., 2012 for discussion of optimization of concentrations). Treatment lasts for 24, 48, or 72 hrs. Using a first sample, a Collagen Invasion Assay (CIA) is performed by seeding the cells on a polymerized layer of collagen-1 prior to treatment (see, e.g., Tselepsis et al., 1998). Using a second sample, a Calcein-AM Dye Transfer Assay (FRAP) is performed (see, e.g., Li et al., 2010). Using a third sample, the cells are lysed with lysis buffer containing protease inhibitors and immunostained with a DSP I/II antibody for western blot analysis. In both treated and untreated cells, the number of tumor cells invading the collagen is determined. The percentage of tumor cells that can transfer calcein-AM is also determined. These determinations are compared to the expression of desmoplakin protein as a function of treatment.

### ii) Effect of AKB-6899 on Desmoplakin Regulation and Inhibition of Metastasis

In this series of experiments, the effect of AKB-6899 on DNA methylation in MDA-MB-231 human TNBC tumor-bearing SCID mice is evaluated. Furthermore, the effect of AKB-6899 on the expression of DNMT-1 and DNMT-3a and the effect of AKB-6899 on the expression of desmoplakin are determined. The effect of AKB-6899 alone or in combination with docetaxel is evaluated on tumor metastasis in the blood, lymph nodes, and lungs.

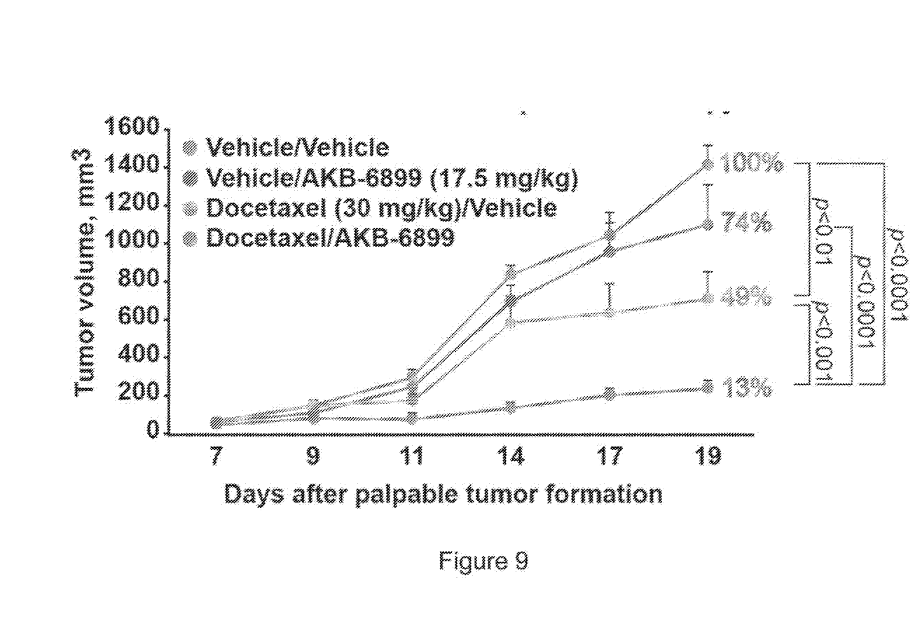

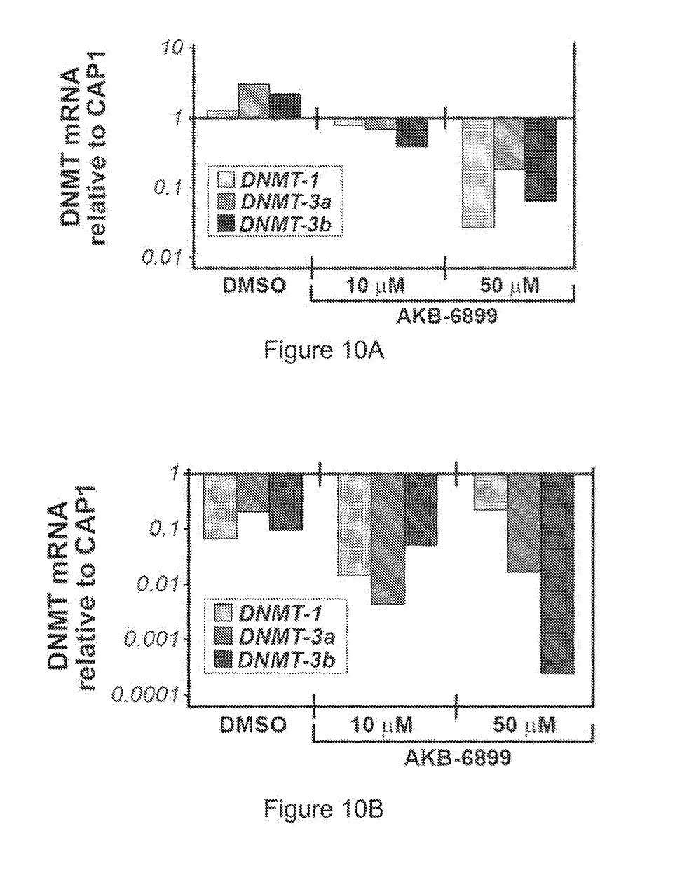

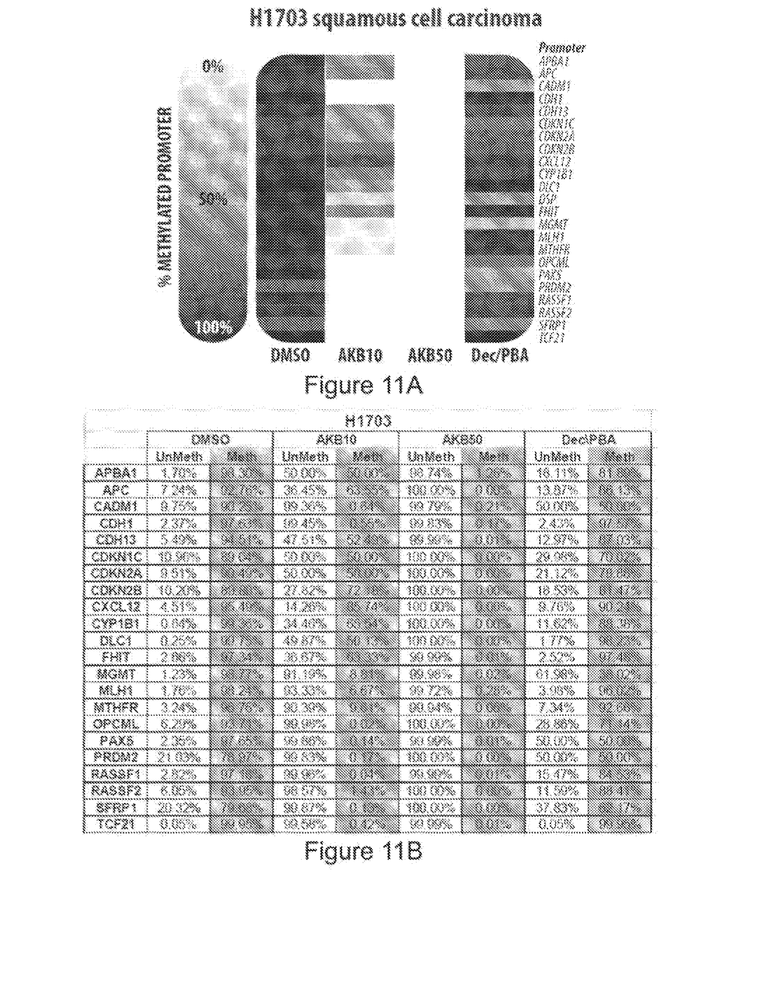

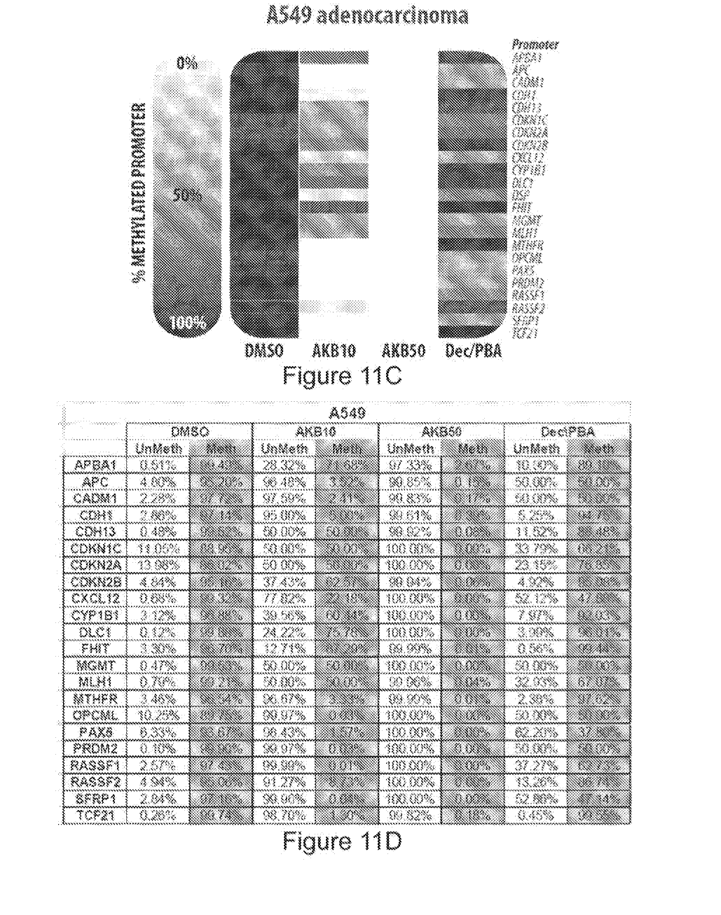

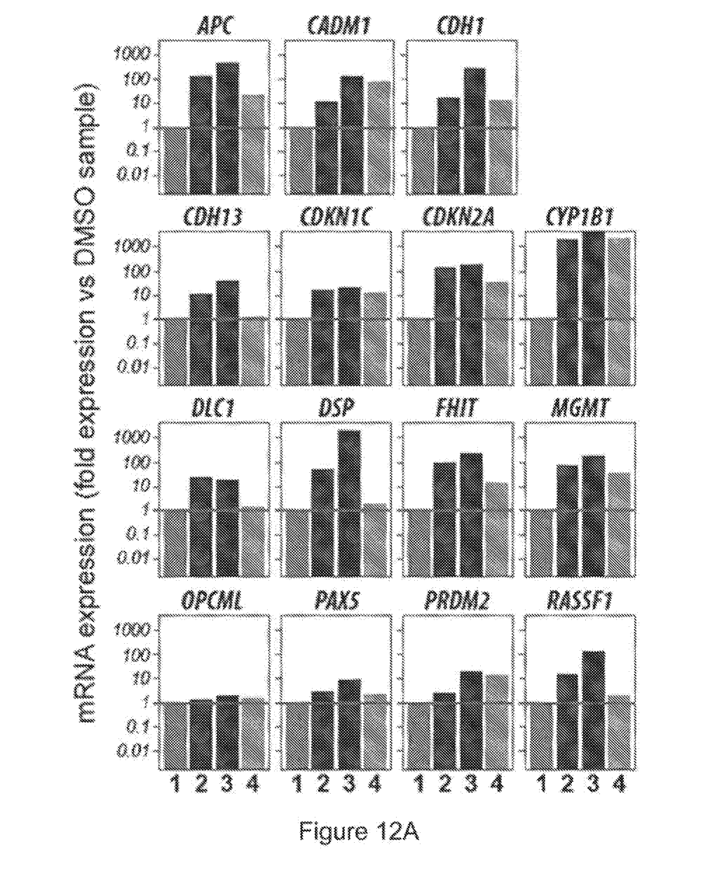

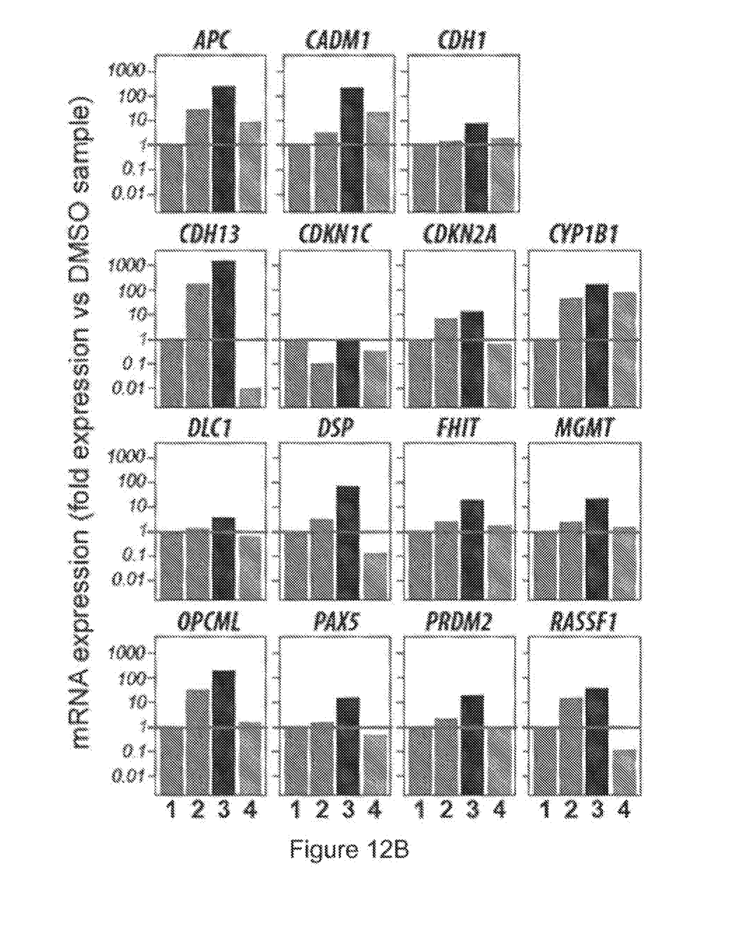

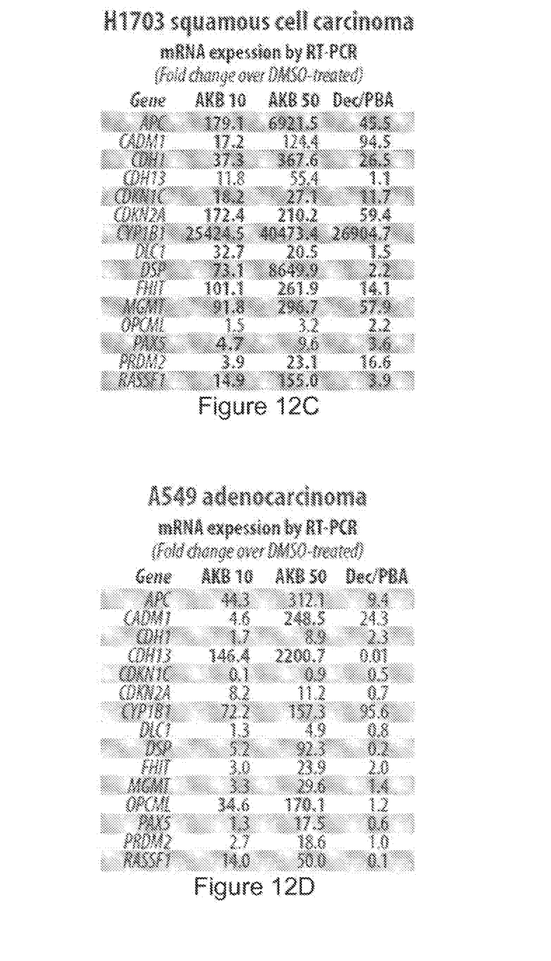

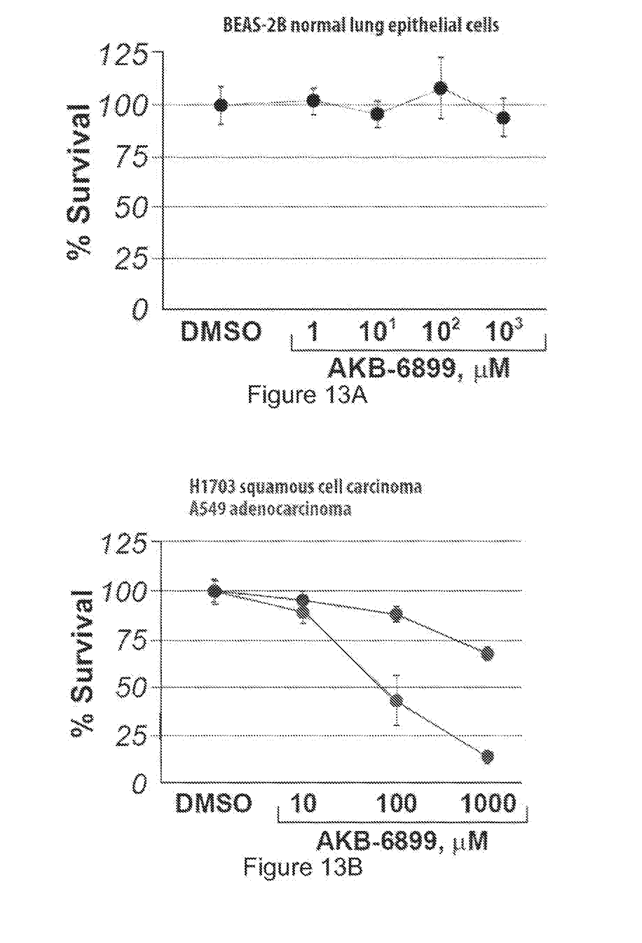

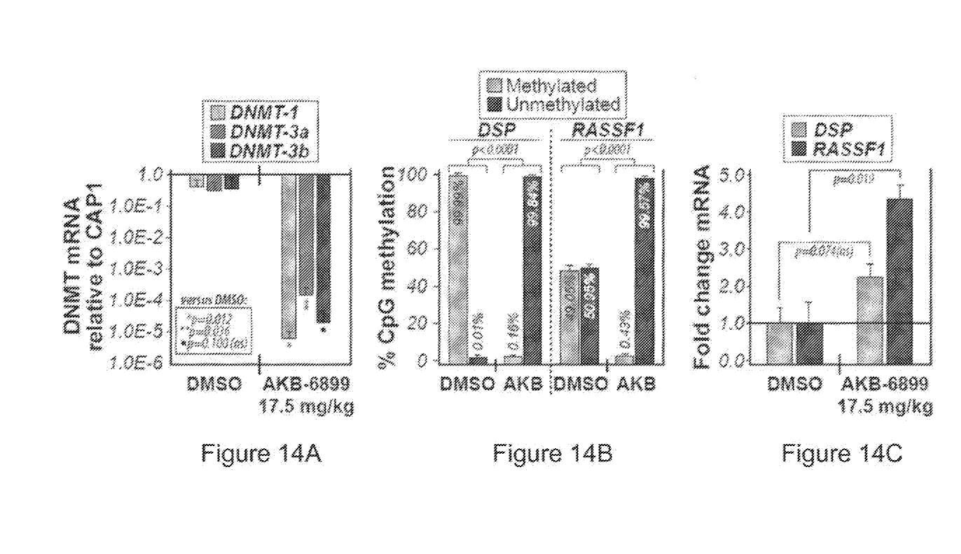

Whether a combination of AKB-6899 with standard chemotherapy like docetaxel reduces human melanoma tumor growth better than docetaxel alone in A375 tumor-bearing SCID mice is determined. FIG. 9 shows that chemotherapy and AKB-6899 can be successfully administered to SCID mouse having a human tumor. FIG. 9 also shows the optimization of the effective dose of both docetaxel and AKB-6899 in vivo (no toxicity to the mice was observed).

Next, whether AKB-6899 treatment increases the mRNA level of DSP and increases DSP protein expression in human breast tumors in SCID mice is determined. Age-matched female SCID mice are orthotopically implanted 1×10⁶ MDA-MB-231 human triple negative breast cancer cells in the number four mammary fat pad. When the tumor is palpable (about 8 days), the mice are randomized into the following treatment groups: (i) untreated (UTX or Utx); (ii) vehicle (DMSO); and (iii) AKB-6899 (17.5 mg/kg given 3× per week). Each treatment is administered by intraperitoneal injection in 100 μL total volume. Treatment continues for approximately 10 weeks or until the mice or the tumors reach removal criteria. One such criterion is a tumor dimension reaching 2 cm. Each day, tumor measures are recorded blindly using calipers. Tumor volume (i.e., length×width×height) and mouse weights are recorded weekly. During each treatment session, morbidity is assessed. After 10 weeks, the mice are humanely euthanized (e.g., CO₂ and cervical dislocation) and the tumors are collected. The tumors are divided into several samples. A first sample is fixed and sectioned for immunohistochemistry using a human DSP I/II antibody for total desmoplakin expression. A second sample is immediately frozen for DNA isolation and subsequent methylation-specific PCR for the DSP promoter. A third sample is used to isolate RNA for determination of the mRNA level of DSP.

Whether AKB-6899 alone or in combination with docetaxel effectively reduces tumor metastases better than docetaxel alone is evaluated. Docetaxel is considered a benchmark therapy for triple negative breast cancer. Age-matched female SCID mice are orthotopically implanted 1×10⁶ MDA-MB-231 human triple negative breast cancer cells in the number four mammary fat pad. When the tumor is palpable (about 8 days), the mice are randomized into the following treatment groups: (i) untreated (Utx); (ii) vehicle 1 (DMSO for AKB-6899) or vehicle 2 (PBS for docetaxel); (iii) docetaxel alone (30 mg/kg 1× per week); (iv) AKB-6899 alone (17.5 mg/kg 3× per week); and (v) combination of docetaxel (30 mg/kg 1× per week) and AKB-6899 (17.5 mg/kg 3× per week). Each treatment is administered by intraperitoneal injection in 100 μL total volume. Treatment continues for approximately 10 weeks or until the mice or the tumors reach removal criteria. One such criterion is a tumor dimension reaching 2 cm. Each day, tumor measures are recorded blindly using calipers. Tumor volume (i.e., length×width×height) and mouse weights are recorded weekly. During each treatment session, morbidity is assessed. After 10 weeks, the mice are humanely euthanized (e.g., CO₂ and cervical dislocation) and the tumors, atrial blood, lymph nodes, and lungs are collected. The tumors are divided into several samples. A first sample is fixed and sectioned for immunohistochemistry using a human DSP I/II antibody for determination of total DSP expression. Perivascular invasion of tumor cells is evaluated by hematoxylin and eosin staining and subjected to pathologist evaluation. A second sample is frozen immediately for DNA isolation and methylation-specific PCR for the desmoplakin promoter. A third sample is used to isolate RNA for desmoplakin mRNA expression. Total RNA is isolated from whole blood, cDNA synthesized, and RT-PCR for the presence of human ERVK6A mRNA in the MDA-MB-231 human tumor cells. A half of the lung is flash frozen in liquid nitrogen and homogenized for total RNA isolation, cDNA synthesis, and RT-PCR for ERVK6A mRNA. To quantify incidence of tumor metastases, the other half of the lung is tied-off and insufflated with PBS, fixed in formalin, stained with hematoxylin, and subjected to bright light microscopy.

The level of human ERVK6A mRNA in the blood, lymph nodes, and lungs is evaluated by RT-PCR, pathologist scoring of tumor cell perivascular invasion, and tumor incidence in the lungs from each of the five treatment groups (i.e., (i) untreated mice, (ii) DMSO or PBS-treated mice (vehicle), (iii) AKB-6899 only, (iv) docetaxel only, and (v) the combination of AKB-6899 and docetaxel treated mice. To confirm DNA hypomethylation, several experiments are conduced: (i) desmoplakin mRNA and protein expression in the tumors is determined, (ii) methylation-specific PCR of the desmoplakin promoter is performed, (iii) RT-PCR for desmoplakin mRNA is performed, and (iv) immunostaining for DSP I/II protein is used.

Statistical analyses are performed. For example, Holm's procedure is used to adjust for multiplicity. Data is analyzed in SAS 9.3 (SAS, Inc, Cary, N.C.). The level of DSP promoter methylation between siRNA and its control under each treatment is compared. Using an n=3 replicates for each condition generates 80% power to detect a 2-fold difference with CV=20% and a=0.025 (1-sided). The level of DSP protein expression and desmosome function for treatment is compared to DMSO, requiring an n=5 replicates/condition (a=0.025/8 for two end points and 4 contrasts). Analysis of variance (ANOVA) is used for hypothesis testing. Metastases between untreated mice, DMSO-treated mice, and AKB-6899-treated mice are compared. An n=10 mice per group generates 80% power to detect a 2.5-fold difference with CV=50% and a=0.025/10 (5 endpoints and 2 contrasts). Linear mixed effect models are used to analyze the repeated tumor size data and ANOVA is used for other endpoints. Metastases are compared between untreated, DMSO-treated or PBS-treated (vehicle), AKB-6899 alone, Docetaxel alone, and the combination of AKB-6899+docetaxel. An n=10 mice per group generates 80% power to detect a 2.5-fold difference with CV=50% and a=0.025/10 (5 endpoints and 2 contrasts). Linear mixed effect models are used to analyze the repeated tumor size data and ANOVA is used for other endpoints.

iii) Determination of Mechanism by which AKB-6899 Causes Reversal of DNA Methylation

AKB-6899 is a specific small molecule inhibitor of prolyl hydroxylase-3 (PHD3), which selectively stabilizes HIF-2α (and not HIF-1α). Tumor cells exposed to intermittent hypoxia (IH) lose HIF-2α but retain HIF-1α (Nanduri et al, 2009). Accordingly, B16F10 tumor-bearing mice were exposed to an IH protocol. Those mice exposed to IH had increased tumor metastases compared to those exposed to intermittent air (p=0.013). Whether DNA methylation regulates hypoxic events through expression and/or suppression of genes that normalize homeostatic mechanisms related to oxygen availability is examined.

Treatment of PyMT mouse breast cancer cells and MDA-MB-231 human triple negative breast cancer cells (which are normally 99.96% and 99.86% hypermethylated, respectively) with AKB-6899 hypomethylated the desmoplakin promoter to 0.0% and 0.38%, respectively. Therefore, the experiments described herein are aimed at (i) determining the mechanism of the AKB-6899-induced hypomethylation, (ii) increasing DSP expression in MDA-MB-231 human triple negative breast cancer cells, (iii) assessing the role of PHD3 and HIF-2α-specific stabilization, (iv) determining how these pathways regulate the DNMTs, and (v) examining the effects of increased DSP expression on desmosome function.

Furthermore, in the experiments described herein, the efficacy of HIF-2α stabilization by AKB-6899 is examined. Specifically, tumor metastases is evaluated by comparing the tumors of MDA-MB-231 human tumor-bearing SCID mice treated with AKB-6899 to the tumors of mice treated with (i) docetaxel alone and (ii) the combination of AKB-6899 and docetaxel. Using the same treatment groups, the level of desmoplakin expression in the tumors is also assessed. A MethylCap-Seq Experimental QC is generated so as to profile changes in global DNA methylation between the tumors treated with the (i) AKB-6899 alone, (ii) docetaxel alone, (iii) decitabine alone, or (iv) various combinations comprising AKB-6899. The methylation profiles are determined for the various treatment groups.

The gene expression signatures from the tumors of PyMT breast tumor-bearing mice that were treated with either vehicle or AKB-6899 were evaluated. The mice treated with AKB-6899 experienced a significant increase in desmoplakin gene expression when compared to the vehicle-treated mice (i.e., 118.5-fold increase in AKB-6899 animals vs. vehicle animals) (FIG. 4A). Methylation-specific PCR on the desmoplakin promoter was performed. The level of hypomethylation was near complete. (FIG. 3A (mouse PyMT breast cancer cells), FIG. 3B (human MDA-MB-231 triple negative breast cancer cells), FIG. 3C (human C8161.9 melanoma tumor cells), and FIG. 3D (human MCF-7 breast cancer cells). To determine whether AKB-6899 increased desmoplakin gene expression in PyMT tumor cells, PyMT tumor cells were treated with AKB-6899 in culture. An upregulation in expression of desmoplakin protein was observed (FIG. 5A). The lack of DSP expression by MDA-MB-231 was confirmed (FIG. 5B). RT-PCR for DNMT-1 and DNMT-3a was performed for both PyMT tumor cells and MDA-MB-231 human tumor cells. AKB-6899 downregulated the DNMT mRNA expression in both PyMT tumor cells and MDA-MB-231 human tumor cells (FIG. 1A (PyMT) and FIG. 1B (MDA-MB-231)).

Next, whether PHD3 inhibition and HIF-2α stabilization inhibits DNA methylation of the desmoplakin promoter in human triple negative breast cancer cells is evaluated. MDA-MB-231 human triple negative breast cancer (TNBC) tumor cells are cultured and are transfected with (i) human siRNA targeting prolyl hydroxylase-3 (PHD3), (ii) human siRNA targeting HIF-2α, or (iii) a scrambled control siRNA. Transfection occurs for 24 hours. (Eubank et al., 2011). The treatment groups are as follows: (i) AKB-6899 (0.25 μM, 2.5 μM, and 25 μM), (ii) DMSO (vehicle), or (iii) 5-aza-2′-deoxycytidine (decitabine) (0.5 μM, 1 μM, and 2 μM) at either 0.5% O₂ or 21% O₂ or with CoCl₂ alone at 21% O₂. Treatment lasts for 24, 48, or 72 hours. Toxicity is evaluated using a Trypan Blue exclusion assay. If necessary (e.g., if a treatment shows signs of toxicity), then the treatment concentrations are adjusted. A first sample is subjected to Trizol homogenization, total RNA purification, cDNA synthesis, and RT-PCR to confirm siRNA silencing (i.e., mRNA knockdown determined by comparing siPHD3 to siScrambled control for PHD3 and comparing siHIF-2a to siScrambled control for HIF-2α). To confirm reduction in respective proteins, a second sample is lysed with cell lysis buffer containing protease inhibitors and lysates are immunostained with PHD3 or HIF-2a antibodies for western blot analysis. A third sample is subjected to pelleting so that DNA isolation is isolated and followed by methylation-specific PCR with primers targeting the desmoplakin promoter.

Whether a HIF-2α stabilization-induced reduction in DNMT transcription causes DNA hypomethylation of the desmoplakin promoter is explored. MDA-MB-231 human triple negative breast cancer tumor cells are cultured and transfected for 24 hours with (i) siRNA targeting human DNMT-1, (ii) siRNA targeting human DNMT-3a, (iii) siRNA targeting human DNMT-3b, (iv) siRNA targeting the combination of human DNMT-1, DNMT-3a, and DNMT-3b, or (iv) scrambled control siRNA. The combinatorial approach to transfecting multiple siRNAs targeting more than one DNMT in CP70 human ovarian cancer cells has been demonstrated. (Leu et al., 2003). Following transfection, the tumor cells are not treated (i.e., untreated) or subjected to one of the following treatment groups: (i) AKB-6899 (0.25 μM, 2.5 μM, and 25 μM), (ii) DMSO (vehicle), or (iii) decitabine (0.5 μM, 1 μM, and 2 μM) at either 0.5% O₂ or 21% O₂ or with CoCl₂ alone at 21% O₂. Treatment lasts for 24, 48, or 72 hours. A first sample is subjected to Trizol homogenization, total RNA purification, cDNA synthesis, and RT-PCR to confirm siRNA silencing. To determine the mRNA level following knockdown, the mRNA level of tumor cells transfected with siDNMT-1, siDNMT-3a, siDNMT-3b, or the combination of siRNAs targeting DNMTs are compared to the mRNA levels of the appropriate siScrambled controls. To confirm a reduction in level of the respective proteins, a second sample is lysed with cell lysis buffer containing protease inhibitors and lysates are immunostained with DNMT antibodies and DSP antibodies for western blot analysis. A third sample is pelleted for DNA isolation followed by methylation-specific PCR with primers targeting the desmoplakin promoter. The absence and/or presence of mRNA for each DNMT and for all DNMTs are compared to the percent DNA methylation of the desmoplakin promoter relative to treatment condition.

Whether HIF-2α stabilization increases desmoplakin protein expression and improves desmosome function in human triple negative breast cancer cells is next examined. MDA-MB-231 human triple negative breast cancer tumor cells are cultured. For 24 hours or 48 hours, the cultured cells are left untreated (Utx) or are treated with DMSO (vehicle), AKB-6899 (0.25 μM, 2.5 μM, and 25 μM), or Decitabine (0.5 μM, 1 μM, and 2 μM) at either 0.5% O₂ or 21% O₂ or with CoCl₂ alone at 21% O₂. A first sample is subjected to a Collagen Invasion Assay (CIA), in which the cells are seeded on a polymerized layer of collagen-1 prior to treatment. (Tselepis et al., 1998). A second sample is subjected to a Calcein-AM Dye Transfer Assay (FRAP), which is performed as described by Li et al., 2010. A third sample is lysed with cell lysis buffer containing protease inhibitors and the lysates are immunostained with a desmoplakin I/II antibody for western blot analysis. The number of tumor cells invading the collagen as well as the percentage of which the tumor cells can transfer calcein-AM are determined and then compared to the expression of desmoplakin protein as a function of the specific treatment.

### iv) Determination of Efficacy of AKB-6899 on Regulation of Desmoplakin and Inhibition of Metastases

The efficacy of HIF-2α stabilization caused by AKB-6899 in MDA-MB-231 human TNBC tumor-bearing SCID mice is determined. The effect of AKB-6899, docetaxel, and the combination of AKB-6899 and docetaxel on tumor metastasis in the blood and lungs is evaluated. A MethylCap-Seq Experimental QC is generated to profile changes in global DNA methylation between the tumors treated with AKB-6899, docetaxel, decitabine, and combinations thereof. These methylation profiles are confirmed using microarray for gene expression.

Whether a combination of AKB-6899 and standard chemotherapy like docetaxel reduces human melanoma tumor growth better than docetaxel alone in A375 tumor-bearing SCID mice is investigated. FIG. 9 confirms the feasibility of administering chemotherapy and AKB-6899 in a human tumor model in SCID mice. FIG. 9 also represents the optimization of the effective dose of both docetaxel and AKB-6899 in vivo (i.e., no toxic effects to the mice were observed).

Whether a combination of AKB-6899 and standard chemotherapy like docetaxel reduces tumor metastases better than docetaxel alone is investigated. Age-matched female SCID are orthotopically implanted with 1×10⁶ MDA-MB-231 human triple negative breast cancer cells into the number four mammary fat pads of mice. Once the tumor is palpable (about 8 days), the mice are randomized into the following treatment groups: (i) vehicle (i.e., PBS for docetaxel, PBS for decitabine, and mineral oil for AKB-6899); (ii) docetaxel alone (30 mg/kg 1× per week); (iii) AKB-6899 alone (17.5 mg/kg 3× per week); (iv) decitabine alone (0.156 mg/kg 2× per week); (v) the combination of docetaxel (30 mg/kg 1× per week) and AKB-6899 (17.5 mg/kg 3× per week); and (vi) the combination of docetaxel and decitabine.

Treatment continues for approximately 10 weeks or until the mice or the tumors reach removal criteria. One such criterion is a tumor dimension reaching 2 cm. Each day, tumor measures are recorded blindly using calipers. Tumor volume (i.e., length×width×height) and mouse weights are recorded weekly. During each treatment session, morbidity is assessed. After 10 weeks, the mice are humanely euthanized (e.g., CO₂ and cervical dislocation) and the tumors, atrial blood (Roda et al., 2012), and lungs (Eubank et al., 2009) are collected. The tumors are divided into several samples. A first sample is fixed and sectioned for immunohistochemistiy using (i) a human desmoplakin I/II antibody for total desmoplakin expression, (ii) a ki-67 antibody for tumor cell proliferation, and (iii) a caspase-3 antibody for tumor cell apoptosis. Perivascular invasion of tumor cells is evaluated by hematoxylin and eosin staining and is subjected to pathologist evaluation. A second sample is immediately frozen for DNA isolation and subsequent methylation-specific PCR of the DSP promoter. A third sample is used to isolate total RNA from whole blood, which is then cDNA synthesized and used in RT-PCR to determine the presence of human ERVK6A mRNA from the MDA-MB-231 human tumor cells. Half of the lung is flash frozen in liquid nitrogen and homogenized for total RNA isolation, cDNA synthesis, and RT-PCR for ERVK6A mRNA expression. To quantify incidence of tumor metastases, the other half of the lung is tied-off and insufflated with PBS, fixed in formalin, stained with hematoxylin, and subjected to bright light microscopy.

Following various treatments, the expression of human ERVK6A mRNA in the blood and lungs is evaluated by RT-PCR, pathologist scoring of tumor cell perivascular invasion, and tumor incidence in the lungs. cDNA hypomethylation and desmoplakin protein expression in the tumors is confirmed by using methylation-specific PCR of the desmoplakin promoter and immunostaining for desmoplakin protein.

The AKB-6899-induced DNA hypomethylation of triple negative breast tumors in SCID mice is next compared to decitabine-induced DNA hypomethylation. Tumor DNA and RNA from the MDA-MB-231 human breast tumor-bearing SCID mice subjected to treatment with (i) AKB-6899, (ii) the combination of AKB-6899 with docetaxel and decitabine alone, and (iii) the combination of AKB-6899 and docetaxel. Methylcap-seq and human microarray gene expression assays are performed as described (Rodriguez et al., 2012). DNA methylation data is analyzed as described in Rodriguez et al., 2012. DNA methylation signatures between treatment groups are confirmed by using the same tumor samples for human microarray gene expression to compare DNA hypermethylation or hypomethylation to functional gene activity.

In these experiments, Holm's procedure is used to adjust for multiplicity. Data is analyzed in SAS 9.3 (SAS, Inc, Cary, N.C.). DSP promoter methylation between siRNA and its control under each treatment is compared. An n=3 replicates for each condition generates 80% power to detect a 2-fold difference with CV=20% and a=0.025 (1-sided). DSP protein expression and desmosome function for treatment is compared to DMSO utilizing an n=5 replicates/condition (a=0.025/8 for two endpoints and 4 contrasts). Analysis of variance (ANOVA) is used for hypothesis testing. The metastases between the various treatments are evaluated (e.g., (i) AKB-6899, (ii) treatment with the combination of AKB-6899 and docetaxel, and (iii) treatment with docetaxel. In this evaluation, an n=10 mice per group generates 80% power to detect a 2.5-fold difference with CV=50% and a=0.025/10 (5 endpoints and 2 contrasts). Linear mixed effect models are used to analyze the repeated tumor size data and ANOVA is used for other endpoints. The levels of gene methylation or expression between AKB-6899 and decitabine are compared by using linear effect model. An n=10 mice/group allows for detection of a 2-fold difference for each gene assuming the standard deviations of the two groups are 0.4 with FDR=0.01 (Jung et al., 2005). Regression models are used to explore the association of methylation and gene expression.

### v) Effect of AKB-6899 on MicroRNA and SnoRNA Expression

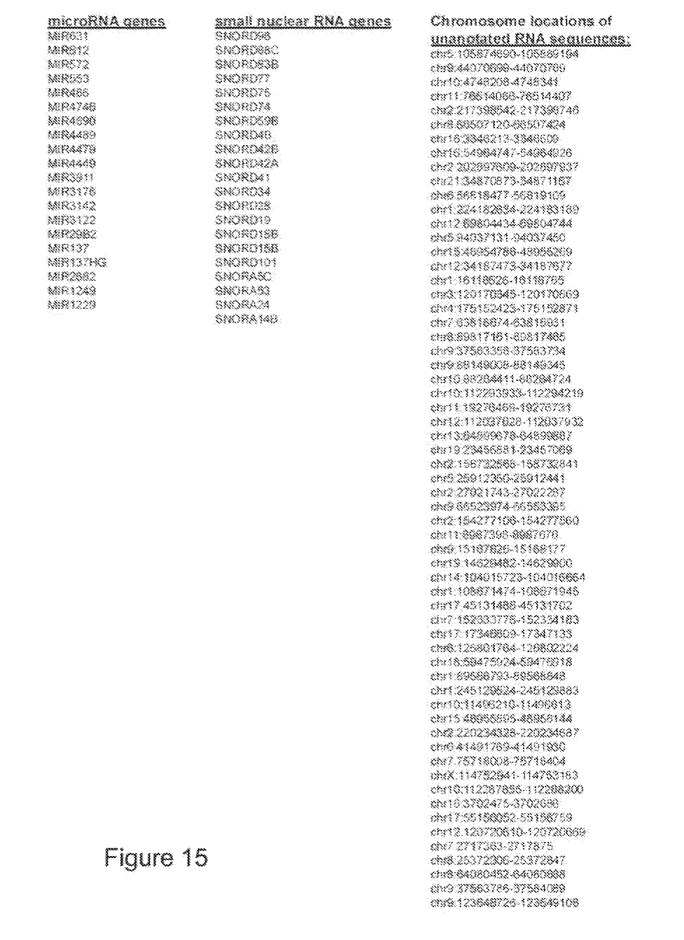

The expression of various microRNAs and snoRNAs was evaluated following administration of AKB-6899 to MDA-MB-231 human breast cancer cells. The data are presented in FIG. 15. In these cancer cells, AKB-6899 increased expression of several microRNAs, some of which target methylation regulators such as the DNMTs. AKB-6899 also regulated about 20 snoRNAs, some of which are known to guide methylation. Finally, the analysis identified over sixty (60) RNA transcripts in the AKB-6899 treated cancer cells, which transcriptions are yet to annotated. The AKB-6899 treated breast cancer cells were compared to DMSO-treated cancer cells.

### E. REFERENCES

Ahmed A, et al. Colocalization of vascular endothelial growth factor and its flt-1 receptor in human placenta. Growth Factors. 2005; 12(3):235-243.

Barekati Z, et al. Methylation signature of lymph node metastases in breast cancer patients. BMC Cancer. 2012; 12:244-2407-12-244.

Chen D, et al. Hypoxia inducible factors-mediated inhibition of cancer by GM-CSF: A mathematical model. Bull Math Biol. 2012; 74(11):2752-2777.

Chimonidou M, et al. DNA methylation of tumor suppressor and metastasis suppressor genes in circulating tumor cells. Clin Chem. 2011; 57:1169-1177.

Clevers H. Wnt/beta-catenin signaling in development and disease. Cell. 2006; 127(3):469-480.

Davies E L, et al. The immunohistochemical expression of desmoplakin and its role in vivo in the progression and metastasis of breast cancer. Eur J Cancer. 1999; 35:902-907.

Derks S, et al. Methylation-specific PCR unraveled. Cell Oncol. 2004; 26(5-6):291-299.

Dusek R L, et al. Desmosomes: New perpetrators in tumour suppression. Nat Rev Cancer. 2011; 11:317-323.

Eubank T D, et al. Granulocyte macrophage colony-stimulating factor inhibits breast cancer growth and metastasis by invoking an anti-angiogenic program in tumor-educated macrophages. Cancer Res. 2009; 69: 2133-2140.

Eubank T D, et al. Opposing roles for HIF-1alpha and HIF-2alpha in the regulation of angiogenesis by mononuclear phagocytes. Blood. 2011; 117:323-332.

Herman J, et al. Methylation-specific PCR: A novel PCR assay for methylation status of CpG islands (DNA methylation/tumor suppressor genes/p16/p15). Proc. Natl. Acad. Sci. USA. 1996. 93:9821-9826.

Leu Y W, et al. Double RNA interference of DNMT-3b and DNMT-1 enhances DNA demethylation and gene reactivation. Cancer Res. 2003; 63:6110-6115.

Li M W, et al. Connexin 43 is critical to maintain the homeostasis of the blood testis barrier via its effects on tight junction reassembly. Proc Natl Acad Sci USA. 2010; 107:17998-18003.

Lin E Y, et al. Progression to malignancy in the polyoma middle T oncoprotein mouse breast cancer model provides a reliable model for human diseases. Am J Pathol. 2003; 163(5):2113-2126.

Matsumoto S, et al. Imaging cycling tumor hypoxia. Cancer Res. 2010; 70(24): 10019-10023.

Maynadier M, et al. Estrogens promote cell-cell adhesion of normal and malignant mammary cells through increased desmosome formation. Mol Cell Endocrinol. 2012; 364:126-133.

Naldini A, et al. Downregulation of hypoxia-related responses by novel antitumor histone deacetylase inhibitors in MDAMB231 breast cancer cells. Anticancer Agents Med Chem. 2012; 12:407-413.

Nanduri J, et al. Intermittent hypoxia degrades HIF-2alpha via calpains resulting in oxidative stress: Implications for recurrent apnea-induced morbidities. Proc Natl Acad Sci USA. 2009; 106:1199-1204.

Ojalvo L S, et al. Gene expression analysis of macrophages that facilitate tumor invasion supports a role for wnt-signaling in mediating their activity in primary mammary tumors. Journal of Immunology. 2010; 184(2):702-712.

Pang H, et al. Epidermal growth factor suppresses induction by progestin of the adhesion protein desmoplakin in T47D breast cancer cells. Breast Cancer Res. 2004; 6:R239-R245.

Roda J M, et al. Hypoxia-inducible factor-2 alpha regulates GM-CSF-derived soluble vascular endothelial growth factor receptor 1 production from macrophages and inhibits tumor growth and angiogenesis. Journal of Immunology. 2011; 187(4): 1970-1976.

Roda J M, et al. Stabilization of HIF-2alpha induces sVEGFR-1 production from tumor-associated macrophages and decreases tumor growth in a murine melanoma model. J Immunol. 2012; 189:3168-3177.

Rodriguez B A, et al. Methods for high-throughput MethylCap-seq data analysis BMC Genomics. 2012; 13 Suppl 6: S14-2164-13-S6-S14.

Szyf M. DNA methylation signatures for breast cancer classification and prognosis. Genome Med. 2012; 4:26.

Tselepis C, et al. Desmosomal adhesion inhibits invasive behavior. Proc Natl Acad Sci USA. 1998; 95:8064-8069.

Watson J A, et al. Epigenetics, the epicenter of the hypoxic response. Epigenetics. 2010; 5:293-296.

Yang L, et al. Desmoplakin acts as a tumor suppressor by inhibition of the Wnt/beta-catenin signaling pathway in human lung cancer. Carcinogenesis. 2012; 33:1863-1870.

What is claimed is:

1. A method of modulating DNA methylation of one or more gene promoters in a subject, comprising:
administering to a subject an effective amount of composition comprising a compound of the formula: 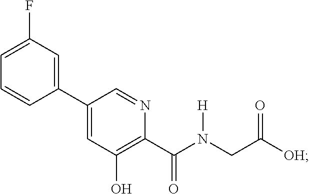 [SMILES: O=C(O)CNC(=O)c1ncc(-c2cccc(F)c2)cc1O]
or a pharmaceutically acceptable salt thereof.

2. The method of claim 1, further comprising determining the methylation status of the one or more gene promoters.

3. The method of claim 2, wherein determining the methylation status of the one or more gene promoters comprises comparing the methylation status of the one or more gene promoters in an affected tissue of the subject to the methylation status of the one or more gene promoters in an unaffected tissue of the subject.

4. The method of claim 2, wherein determining the methylation status of the one or more gene promoters comprises measuring the percent methylation of the one or more promoters.

5. The method of claim 1, wherein, prior to the administering step, the one or more gene promoters are hypermethylated.

6. The method of claim 1, wherein, after the administering step, there is a change in the methylation status of the one or more gene promoters.

7. The method of claim 6, wherein the change in methylation status comprises a decrease in the percent methylation of the one or more gene promoters.

8. The method of claim 6, if the desired methylation status is not achieved, then the method further comprises repeating the administration of an effective amount the composition.

9. The method of claim 6, wherein the desired methylation status is a decrease in the percent methylation of the one or more gene promoters.

10. The method of claim 8, further comprising determining the methylation status of the one or more gene promoters.

11. The method of claim 1, wherein the composition inhibits the expression of one or more DNA methyltransferases.

12. The method of claim 1, wherein the composition inhibits the expression of one or more histone deacetylases.

13. The method of claim 1, wherein the composition inhibits the expression of one or more prolyl hydroxylases.

14. The method of claim 1, wherein administering comprises intraperitoneal administration.

15. The method of claim 1, wherein administering comprises oral administration.

16. The method of claim 1, wherein administering comprises intravenous administration.

17. The method of claim 1, wherein the one or more gene promoters comprises a desmoplakin (DSP) gene promoter, a c-myc gene promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a cytochrome P450, family 1, subfamily B, polypeptide 1 (CYP1B1) promoter, a deleted in liver cancer 1 (DLC1) promoter, an E-cadherin (CDH1) promoter, a fragile histidine triad (FHIT) promoter, a H-cadherin (CDH13) promoter, an O-6-methylguanine-DNA methyltransferase (MGMT) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, an Adenomatous polyposis coli (APC) promoter, an Amyloid beta A4 precursor protein-binding family A member 1 (APBA1) promoter, a cell adhesion molecule 1 (CADM1) promoter, a chemokine (C—X—C motif) ligand 12 (CXCL12) promoter, a methylenetetrahydrofolate reductase (NAD(P)H) (MTHFR) promoter, a mutL homolog 1 promoter, nonpolyposis type 2 (MLH1) promoter, a Ras association (RalGDS/AF-6) domain family member 2 (RASSF2) promoter, a secreted frizzled-related protein 1 (SFRP1) promoter, a transcription factor 21 (TCF21) promoter, or a vascular endothelial growth factor-A (VEGF-A) promoter.

18. The method of claim 1, wherein the one or more gene promoters comprises an estrogen receptor gene (ER) promoter, a breast cancer 1 gene (BRCA1) promoter, an epithelial cadherin gene (E-cad) promoter, a TMS1 gene promoter, an insulin-like growth factor binding protein 7 gene (IGFBP7) promoter, a p16 promoter, a rentioic acid receptor gene (RARβ2) promoter, or a Ras association (RalGDS/AF-6) domain family member 1 gene (RASSF1A) promoter.

19. The method of claim 1, wherein the subject is unhealthy.

20. The method of claim 1, wherein the subject has cancer.

21. The method of claim 19, wherein the cancer is breast cancer and the one or more gene promoters comprises a desmoplakin (DSP) promoter.

22. The method of claim 19, wherein the breast cancer is triple negative breast cancer.

23. The method of claim 19, wherein the cancer is melanoma and the one or more gene promoters comprises a desmoplakin (DSP) promoter.

24. The method of claim 19, wherein the cancer is cervical cancer and the one or more gene promoters comprise a desmoplakin (DSP) promoter or a c-Myc promoter.

25. The method of claim 19, wherein the cancer is lung cancer and the one or more gene promoters comprise a desmoplakin (DSP) promoter, an Adenomatous polyposis coli (APC) promoter, an Amyloid beta A4 precursor protein-binding family A member 1 (APBA1) promoter, a cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

26. The method of claim 19, further comprising administering one or more anti-cancer agents.

27. A method of modulating DNA methylation of one or more gene promoters in a subject, comprising:
identifying a subject in need of treatment by determining the methylation status of one or more gene promoters; and
administering to a subject an effective amount of composition comprising a compound of the formula: 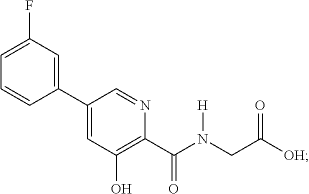 [SMILES: O=C(O)CNC(=O)c1ncc(-c2cccc(F)c2)cc1O]
or a pharmaceutically acceptable salt thereof.

28. The method of claim 27, wherein determining the methylation status of the one or more gene promoters comprises comparing the methylation status of the one or more gene promoters in an affected tissue of the subject to the methylation status of the one or more gene promoters in an unaffected tissue of the subject.

29. The method of claim 27, wherein determining the methylation status of the one or more gene promoters comprises measuring the percent methylation of the one or more promoters.

30. The method of claim 27, wherein, prior to the administering step, the one or more gene promoters are hypermethylated.

31. The method of claim 27, wherein, after the administering step, there is a change in the methylation status of the one or more gene promoters.

32. The method of claim 31, wherein the change in methylation status is a decrease in percent methylation of the one or more promoters.

33. The method of claim 31, wherein if the desired methylation status is not achieved, then the method further comprises repeating the administration of an effective amount the composition.

34. The method of claim 33, further comprising determining the methylation status of the one or more gene promoters.

35. The method of claim 27, wherein the subject is unhealthy.

36. The method of claim 27, wherein the subject has cancer.

37. The method of claim 35, wherein the affected tissue is a tumor or a cancer and wherein the unaffected tissue is not a tumor or a cancer.

38. The method of claim 27, wherein the composition inhibits the expression of one or more DNA methyltransferases.

39. The method of claim 27, wherein the composition inhibits the expression of one or more histone deacetylases.

40. The method of claim 27, wherein the composition inhibits the expression of one or more propyl hydroxylases.

41. The method of claim 27, wherein administering comprises intraperitoneal administration.

42. The method of claim 27, wherein administering comprises oral administration.

43. The method of claim 27, wherein administering comprises intravenous administration.

44. The method of claim 27, wherein the one or more gene promoters comprises a desmoplakin (DSP) gene promoter, a c-myc gene promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a cytochrome P450, family 1, subfamily B, polypeptide 1 (CYP1B1) promoter, a deleted in liver cancer 1 (DLC1) promoter, an E-cadherin (CDH1) promoter, a fragile histidine triad (FHIT) promoter, a H-cadherin (CDH13) promoter, an O-6-methylguanine-DNA methyltransferase (MGMT) promoter, an opioid binding protein/cell adhesion molecule-like (OPCML) promoter, a paired box 5 (PAX5) promoter, a PR domain containing 2, with ZNF domain (PRDM2) promoter, a Ras association (RalGDS/AF-6) domain family member 1 (RASSF1) promoter, an Adenomatous polyposis coli (APC) promoter, an Amyloid beta A4 precursor protein-binding family A member 1 (APBA1) promoter, a cell adhesion molecule 1 (CADM1) promoter, a chemokine (C—X—C motif) ligand 12 (CXCL12) promoter, a methylenetetrahydrofolate reductase (NAD(P)H) (MTHFR) promoter, a mutL homolog 1, a nonpolyposis type 2 (MLH1) promoter, a Ras association (RalGDS/AF-6) domain family member 2 (RASSF2) promoter; a secreted frizzled-related protein 1 (SFRP1) promoter, a transcription factor 21 (TCF21) promoter, or a vascular endothelial growth factor-A (VEGF-A) promoter.

45. The method of claim 27, wherein the one or more gene promoters comprises an estrogen receptor gene (ER) promoter, a breast cancer 1 gene (BRCA1) promoter, an epithelial cadherin gene (E-cad) promoter, a TMS1 gene promoter, an insulin-like growth factor binding protein 7 gene (IGFBP7) promoter, a p16 promoter, a rentioic acid receptor gene (RARβ2) promoter, or a Ras association (RalGDS/AF-6) domain family member 1 gene (RASSF1A) promoter.

46. The method of claim 35, wherein the cancer is breast cancer and the one or more gene promoters comprises a desmoplakin (DSP) promoter.

47. The method of claim 46, wherein the cancer is triple negative breast cancer.

48. The method of claim 35, wherein the cancer is melanoma and the one or more gene promoters comprises a desmoplakin (DSP) promoter.

49. The method of claim 35, wherein the cancer is cervical cancer and the one or more gene promoters comprises a desmoplakin (DSP) promoter or a c-Myc promoter.

50. The method of claim 35, wherein the cancer is lung cancer and the one or more gene promoters comprise a desmoplakin (DSP) promoter, an Adenomatous polyposis coli (APC) promoter, an Amyloid beta A4 precursor protein-binding family A member 1 (APBA1) promoter, an cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

51. The method of claim 35, further comprising administering one or more anti-cancer agents.

52. A method for treating a subject diagnosed with or suspected of having a disease or disorder characterized by DNA hypermethylation, comprising:
administering to a subject an effective amount of a composition comprising a compound of the formula: 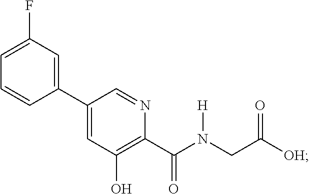 [SMILES: O=C(O)CNC(=O)c1ncc(-c2cccc(F)c2)cc1O]
or a pharmaceutically acceptable salt thereof.

53. The method of claim 52, further comprising determining the methylation status of one or more gene promoters.

54. The method of claim 53, wherein determining the methylation status of the one or more gene promoters comprises measuring the percent methylation of the one or more gene promoters.

55. The method of claim 53, wherein determining the methylation status of the one or more gene promoters comprises comparing the methylation status of the one or more gene promoters in an affected tissue of the subject to the methylation status of the one or more gene promoters in an unaffected tissue in a subject.

56. The method of claim 52, wherein, prior to the administering step, the one or more gene promoters are hypermethylated.

57. The method of claim 52, wherein, after the administering step, there is a change in the methylation status of the one or more gene promoters.

58. The method of claim 57, wherein the change in methylation status comprises a decrease in the percent methylation of the one or more gene promoters.

59. The method of claim 57, wherein if the desired methylation status is not achieved, then the method further comprises repeating the administration of an effective amount the composition.

60. The method of claim 59, wherein the desired methylation status comprises a decrease in the percent methylation of the one or more gene promoters.

61. The method of claim 59, further comprising determining the methylation status of the one or more gene promoters.

62. The method of claim 52, wherein the composition inhibits the expression of one or more DNA methyltransferases.

63. The method of claim 52, wherein the composition inhibits the expression of one or more histone deacetylases.

64. The method of claim 52, wherein the composition inhibits the expression of one or more prolyl hydroxylases.

65. The method of claim 52, wherein administering comprises intraperitoneal administration.

66. The method of claim 52, wherein administering comprises oral administration.

67. The method of claim 52, wherein administering comprises intravenous administration.

68. The method of claim 52, wherein the disease or disorder characterized by DNA hypermethylation is not cancer.

69. The method of claim 52, wherein the subject is unhealthy.

70. The method of claim 52, wherein the subject has cancer.

71. The method of claim 69, wherein the cancer is breast cancer and the one or more gene promoters comprises a desmoplakin (DSP) promoter.

72. The method of claim 71, wherein the cancer is triple negative breast cancer.

73. The method of claim 69, wherein the cancer is melanoma and the one or more gene promoters comprises a desmoplakin (DSP) promoter.

74. The method of claim 69, wherein the cancer is cervical cancer and the one or more gene promoters comprise a desmoplakin (DSP) promoter or a c-Myc promoter.

75. The method of claim 69, wherein the cancer is lung cancer and the one or more gene promoters comprise a desmoplakin (DSP) promoter, an Adenomatous polyposis coli (APC) promoter, an Amyloid beta A4 precursor protein-binding family A member 1 (APBA1) promoter, a cell adhesion molecule 1 (CADM1) promoter, an E-cadherin (CDH1) promoter, a H-cadherin (CDH13) promoter, a cyclin-dependent kinase inhibitor 1C (CDKN1C) promoter, a cyclin-dependent kinase inhibitor 2A (CDKN2A) promoter, a cyclin-dependent kinase inhibitor 2B (CDKN2B) promoter, a chemokine (C—X—C motif) ligand 12 (CXCL12) promoter, a cytochrome P450, family 1, subfamily B, polypeptide 1 (CYP1B1) promoter, a deleted in liver cancer 1 (DLC1) promoter, a fragile histidine triad (FHIT) promoter, an O-6-methylguanine-DNA methyltransferase (MGMT) promoter, a mutL homolog 1, colon cancer, nonpolyposis type 2 (MLH1) promoter, a methylenetetrahydrofolate reductase (NAD(P)H) (MTHFR) promoter, an opioid binding protein/cell adhesion molecule-like (OPCML) promoter, a paired box 5 (PAX5) promoter, PR domain containing 2, with ZNF domain (PRDM2) promoter, a Ras association (RalGDS/AF-6) domain family member 1 (RASSF1) promoter, a Ras association (RalGDS/AF-6) domain family member 2 (RASSF2) promoter, a secreted frizzled-related protein 1 (SFRP1) promoter, or a transcription factor 21 (TCF21) promoter.

76. The method of claim 69, further comprising administering one or more anti-cancer agents.

77. A method for decreasing c-myc expression in a subject, comprising:
administering to a subject an effective amount of a composition comprising a compound of the formula: 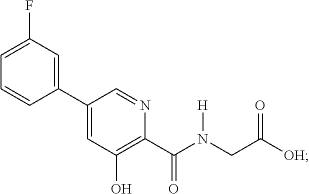 [SMILES: O=C(O)CNC(=O)c1ncc(-c2cccc(F)c2)cc1O]
or a pharmaceutically acceptable salt thereof.

78. The method of claim 77, furthering comprising altering the methylation status of the c-myc promoter.

79. The method of claim 77, further comprising determining the methylation status of the c-myc promoter.

80. The method of claim 79, wherein determining the methylation status of the c-myc promoter comprises measuring the percent methylation of the c-myc promoter.

81. The method of claim 77, wherein, prior to the administering step, the c-myc promoter is hypermethylated.

82. The method of claim 77, wherein, after the administering step, there is a change in the methylation status of the c-myc promoter.

83. The method of claim 82, wherein the change in the methylation status of the c-myc promoter comprises a decrease in the percent methylation of the c-myc promoter.

84. The method of claim 77, wherein the subject is unhealthy.

85. The method of claim 77, wherein the subject has cancer.

86. The method of claim 84, wherein the cancer is cervical cancer.

87. The method of claim 77, wherein a decrease in c-myc expression inhibits metastases in the subject.

88. The method of claim 77, wherein the composition inhibits the expression of one or more DNA methyltransferases.

89. The method of claim 77, wherein the composition inhibits the expression of one or more histone deacetylases.

90. The method of claim 77, wherein the composition inhibits the expression of one or more prolyl hydroxylases.

91. A method for increasing desmoplakin expression in a subject, comprising:
administering to a subject an effective amount of a composition comprising a compound of the formula: 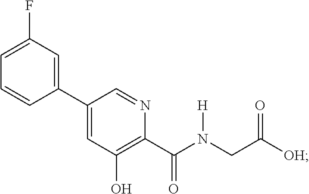 [SMILES: O=C(O)CNC(=O)c1ncc(-c2cccc(F)c2)cc1O]
or a pharmaceutically acceptable salt thereof.

92. The method of claim 91, furthering comprising altering the methylation status of a desmoplakin promoter.

93. The method of claim 91, further comprising determining the methylation status of the desmoplakin promoter.

94. The method of claim 93, wherein determining the methylation status of the desmoplakin promoter comprises measuring the percent methylation of the desmoplakin promoter.

95. The method of claim 91, wherein the gene expression of desmoplakin is increased.

96. The method of claim 91, wherein the protein expression of desmoplakin is increased.

97. The method of claim 91, wherein the subject is unhealthy.

98. The method of claim 91, wherein the subject has cancer.

99. The method of claim 97, wherein the cancer is breast cancer.

100. The method of claim 99, wherein the breast cancer is triple negative breast cancer.

101. The method of claim 97, wherein the cancer is melanoma.

102. The method of claim 97, wherein the cancer is cervical cancer.

103. The method of claim 97, wherein the cancer is lung cancer.

104. The method of claim 97, further comprising administering one or more anti-cancer agents.

105. The method of claim 91, wherein administering comprises intraperitoneal administration.

106. The method of claim 91, wherein administering comprises oral administration.

107. The method of claim 91, wherein administering comprises intravenous administration.

108. The method of claim 91, wherein an increase in desmoplakin expression inhibits metastases in the subject.

109. A method for inhibiting metastases in a subject, comprising:
administering to a subject an effective amount of a composition comprising a compound of the formula: 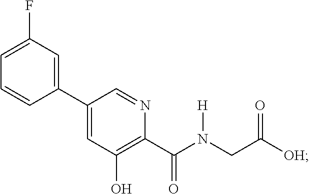 [SMILES: O=C(O)CNC(=O)c1ncc(-c2cccc(F)c2)cc1O]
or a pharmaceutically acceptable salt thereof; and
modulating the DNA methylation status of one or more genes promoters.

110. The method of claim 109, wherein modulating the DNA methylation status of one or more gene promoters comprises changing the methylation status of one or more gene promoters.

111. The method of claim 110, wherein changing the methylation of the one or more gene promoters comprises reducing the percent methylation of the one or more promoters.

112. The method of claim 109, wherein the one or more genes comprise a desmoplakin gene.

113. The method of claim 109, wherein the gene expression of desmoplakin is increased.

114. The method of claim 109, wherein the protein expression of desmoplakin is increased.

115. The method of claim 109, wherein the subject is unhealthy.

116. The method of claim 109, wherein the subject has cancer.

117. The method of claim 115, wherein the cancer is breast cancer.

118. The method of claim 117, wherein the breast cancer is triple negative breast cancer.

119. The method of claim 115, wherein the cancer is melanoma.

120. The method of claim 115, wherein the cancer is cervical cancer.

121. The method of claim 115, wherein the cancer is lung cancer.

122. The method of claim 115, further comprising administering one or more anti-cancer agents.

123. A composition for treating cancer, comprising: an effective amount of a compound of the formula: 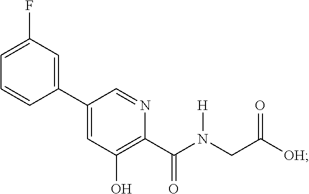 [SMILES: O=C(O)CNC(=O)c1ncc(-c2cccc(F)c2)cc1O]
or a pharmaceutically acceptable salt thereof; and one or more chemotherapeutic agents.
